SEQUENCE LISTING (25 sequences)

SEQ ID NO: 1 - DNA, 7536 bp, Danio rerio
  misc_feature: Type I Fatty Acid Synthase (FAS) coding sequence
atggaggatatcgttatagctggaatttcaggtcgccttcctgagtcgaacaatttggaagaattttggcagaaccttttcaatggagtggacatggtcacagaagacgacaggcggtggaaaccaggtctgtatggccttcccagaagaaatggaaaactaaaagaaatcgacagatttgacgcagccttctttggcgtgcatcccaaacaggcccacaccatggacccacagctcaggcttatgctagagatatcgtacgaggccatcgttgatggaggcataaacccggtgtccatgcgtggcagcaaaactggcgtctacattggagtgagcggctcagaggcaggagaggccttcagtaaagacccggaggagctgctgggatacagcatgacaggatgccagcgagccatgtttgccaaccgcttgtcctacttctttgatttcaatggtcccagcacagccattgacacagcgtgttcctctagtctgctggcactggaaaacgccttcaatgctattcgccatggccaatgtgatgccgcactcattggcggagtgaacctgctgctcaagcccaacacctcagtgcagttcatgaaactgggcatgctcagtcctgaaggaacctgcaagtcatttgatgcctcaggaaatgggtattgtcgctcagaggctgctgttgcagtactcttgacgaagaagtcaatggctaagagaatctatgccacggttctcaatgctggcaacaacacagacggctacaaagagcaaggtgtgacgttcccttcaggtgagatgcaacaaagactggtccgttctctttaccaggaggccaacatctcacctgagcaggtggagtacattgaggcccacggcaccggtactaaggttggagatcctcaggaagtgaacggcattgtgagcgtgttctgtcagtcaccgagggatcctcttctgattggttcaaccaaatccaacatgggacacccggagcctgcctctggattggcagctcttgccaaggtggttttgtctcttgaacatggcgtctgggctcccaatgttcactttaatgaaccaaaccctgacattcctgcactgacagatggtcggttgtgtgtggtgtcgaagcctacaccagtccgcggtggcatcgtgggcatcaactcatttgggtttggaggctcaaatgtccatgtgatcctgagtccacatgtttcagattcatcggataaaacaccagctccttcagtccccagactcttgcaggcctcgggccgcactgaggaggccgtcactgccctcttcagcaacgcccagcagcatcaggagaactccagctacctgtctctgctgaatgatgtgtctggggtcccaacggcaggaatgccatacaggggctacacactgatcggggcgcagggggagctaacagaggttcagcaaacacagcccacaccaagaccactctggtacatttgctcaggtatgggcacgcagtgggcaggaatgggccaaagcctgatgcagctgtctgagttccgggagtccatcaagcgctcagacatcgctttaaaggacactgggctgtgtgtgagccgcctgctcatggaagctgatgagagcacttttgaagacacggttcacgcatttgtgggtctggcagctattcaggttgcccagattgatatgctacggaagatgggtctggagcctaacgggattgtgggtcactctgtcggagagttggcttgtggctacgccgacggttctttgagtcacagtgaggccattctggctgcttactggagaggacgctgcattaaagaggccaatctgccaccgggaggcatggctgctgtgggcctgacgtgggaagagtgcaaagctcagtgtccacagggtgttgtaccagcctgtcataatgctgaggacacagtgaccatctcaggccctcaggactctgtcagcaagtttgtcgcccagttaaaagagagcggtgtgtttgccaaagaagtgcgcagtgccggtgtggctttccactcgtattacatggcctccatcgctccagctctcctgtctgcactacagaaggtaataaagagcccaaggccccgcacagctcgctggatcagcacatctatcccgcagtcagactgggagagcccactggccctctattcttcagccgagtaccacgtcaataacctggtcagccctgtgctcttccaggagggactgaatcatgtgccggataatgctgtggttgtagaaatcgccccgcatgccttgctgcaggccatcctgaagcgaagcctgaaacccacctgttccatcctccccctgatgaaaagaggacatgccaacaatctggagttcttcctctctcatgtgggcaaggtctacatgaatggaattaacgtggacagtaacaaactgtatccagctgtgaaataccctgttcccagaggaacccccctcatttccccttacattcagtgggatcattcccagagctgggatgtcccaaaggtggaagatttccctgctggctcagggggatccacttctgccactgtgtacaatattgacatgaatccagagtctcctgattactacatgattggtcattgcattgatggacgagtgctttacccagccacaggatacctggttctagcctggaggactctgatgagatccctgggcacggtcatggaccacacaccggttacttttgaagacgtcaccatccatagagccaccatcctgcccaagacagggtccgtccagctggaagtgcgacttatgcctgcgactaaccgctttgaggtttcagaaaatggaaatcttgcagtcagtggtaaggtgagtgtcttggaggactctggacttgatgcttttcatgctgagctgaataaaccaataactgcagacaatgaagatcccaaactgcgtttaaaatctggagacatctacaaagagttacgtctgcgtggatacgactatggcaaaaccttccagggaatccttgaatctaacaatgctggtgacagtggaaaacttcactggacaggtaactgggtgacctttttagacaccatgctgcagatgattgtggttgggctgcctggccgcagcttgagactgcccacacgtatccggtctgtgtgtgtggaccccaaacttcatgaggagagggtgaacgactatgaaggagaccaaaaagctgtcaatgtatttgttaaccgatgtctggacaacataaccgctggaggggttcagatctgtgggcttcacgctactgcagcaccccgtaggcagcaacagcagacgccaccgactttggaggagtttgtctttgttccctatgaagatgctgactgcctgagaaccaatgagaagcttgctgaccagctgaggcattgcaaaggtctagtgcagcgtctgcagagaaagcttgccaaacagggagtgaaaatctccatccctgggttagaaggtgcatctgagggccagctgattgaagcggaggcagaaaaaggtcttttgcgtctgctgtcggttctgtgtggactggaactcaatgggaacctgcgctcagagctggaacagacggtgcagaaggagagagactgtctcctgcaagaccctctgctcaacggcctgctggactcccaggccttacgccactgcttggacactgcgcttgaaaacagcactcctggaaaattcaaagtcctggaggctcttgcagcagatggccgtgtgttttctcaggctgtcagtctcctcaatattcagccgatgttgcgtcttgactacactgcttcagacatctctgctgatcagctttctgctcagcaaagctccctggaggagcagggcatctcaactgctcagtgggaccctcttcaggggccagtaacaggtggactggacggagctgatctggttgtgtgcaactgtgcagttggatcagcaaccaatccagcacttctgatcgagaatctgacctcagctgcgagggaagggggctttattttactccacactttactcagaggagacactcttggggagacggtagccttcctcacatctcaaaacaacaggaagggtttgttgactcagactgaatgggaggagcttttccagaaggcctctttaaatgtggtcatgctcagaaaatcttactatggctctgctctcttcctctgccggcgcagccaacaatcctctcaaaaacagccgatacatattttcgtagaccctactgactacaaatgggttgaaactttaaagagcactttagctgagtcatctgatatcccagtttggctcattgccaccaagggccataatggagtggtgggaatggtgaactgcctcagacaggagcctggaggcaaccgtatacggtgtacatttgtttctaatttaagtaagggtgctgctgttccctcccttcttcctaatgaaaaggtcatgaaggcattattaaagaaggacctggttatgaatgtgcaccgggatggactctggggtgtttttagacaccagttgttaacacaagatctcagcgaggagttgacggagcaggcttacgtgaacgtcctgactcgtggtgacctctcatctctgcgctggatcgcttccccactgcgtcattttgtcgcgtccagtcccaacgtgcagctctgccgcgtttactactcctccctcaacttcagagacatcatgctggccactggcaaactgcctccagatgccatccctggagacgtagcgctgcagcagtgcatgctgggaatggagttttcagggcgagaccccagtgggcgacgtgtaatgggtttgctgccagctaaaggccttgccacctgtgtggatgcagacaaacgcttcctttgggacgttccatccagctggacattggagcaggctgcctctgtgccagtggtgtatgccaccgcatattattccctggtggtgcgaggccggctgaggcctggagaaagtgtgctgatccattcaggctctggaggagtgggacaggccgcaattgctattgcacttagcatgcgctgtcgagtcttcacaactgtgggctctaaagagaagaagcagtatctgcaggaaagattcccacaacttacagctgaatcttttgccaactccagggattcttcatttgagcagcacgtaatgctcaacactcagggcaaaggagtagacttggtactaaactctcttgctgaagaaaagctgcaggccagcttgcgttgtttggctagacatggccgttttctggagattggcaaatatgatctttccaacaacacaccactaggtatggctctgttcctaaagaacgtggctttccatgggatcctgcttgatgctttgtttgaagaagggaacagggaatgggaagaggtgtctgatctgctgaagaagggaatctccagtggagttgttcagccgttgcgtaccacagtctttgagagaaaccaagtggaggaagctttccgctacatggctcaaggcaaacacatcggaaaagtgctgctacaggtgcgttcagaggagtcaagcagctctggtcctgcagtgtctgctctctctatccctgcaatctgccgaactttctgtcccgcttctctatcgtacatcatcactggaggcctgggtggcttcgggctggaactggcccagtggctcacagagagaggagctcgtaaactggtgctgacgtcacggtctggcatccgcaacggttatcaggccaagcgtgttcgtgagtggcaggccatgggcatccaggtcctcgtgtccaccagtgatgtcagcaccctggatggcacagaacggctcatcactgaggcctgccgcttgggtccagttggcggcatcttccacctggccatggtccttaaagatggtatgctggagaatctgaccccacaggagttcattgaagtcaacagaccgaaatatgatggaaccatcaatctcgacagtgtgactaggcagaagtgcccgcagctccagcagtttgtggtgttttcctctgtcagctgtggtcgtggtaatgccggccaaagcaactacggcttcgccaactccaccatggagcgaatctgtgaacagcgccgtcaagaaaacctgccaggcctggccgtacaatggggtgccattggagacgtgggtgtcgttttggagaccatgggtggcaacgatgctgtgataggtggcacgctgccccagcgcatgtcttcctgtttggaagtgctggatcgcttcttgtgccagcaaaggcctgtgatgtccagcttcgtgttggcagagaaagtggtggtcactaaaggagaaggaagtggacagaaggacctggtggaggctgtggctcatattctgggtgtgcgtgacgtgaacagtctgaatgctgacgcctcattggctgatctgggtctggattcactgatgggtgttgaggtccggcagacgctggagagggactacgatatcgtgatggcaatgagggaaatccgacagttgaccataaacaagctgcgggagctttccaagcaatcaggagggaaagaggagtctcctgtaaagcgttctggtgctcaggctctgttggagtcagatctgagccggatgctggtgaacccagatggccccacgatggccccgctgaacgaggtccagagtgcagagaggcctttgttcctggttcaccccattgagggctccatcgccgccttccgcaccctcactgccaagctgagtgtgccgtgctatggactgcagtgcactaaagctgctcctcttgacagtatccagagtcttgctgcgtactatgtggagtgtgtgaggcaggttcagctggagggcccataccgcatcgctggatactcgttcggggcgtgtgtggcgtttgagatgtgctcacagctgcagttggccaaatgccccgtggaatatctgttcctgttcgatggctcgcactcttatgtggcagcatatacacagagttacagagccaaactgactcctggcaaagaggctgaagctgagactgaagctctgtgtgctttcatccaacagttcaccggcattgagtataacaagctgttggagacgttgctgcctctgtcagacctcgaggctcgtgtggacaaggctgttgatctgatcacatccagccataagaacgtcagccgagatatgctccattttgctgcttctacattttaccacaaactcaaggctgccgaccgctacgttcccacttccaaatatcacggcaatgtcaccctactgagagccaaggccagcagcgagtacggagatggattgggatcagactacaaactgcacgaggtgtgtgacgggaaggtgtcagttcatgtgatcgaaggcgaccaccggaccttcctggagggtgaaggtgttgagtctatttccagcatcatccacagctcactgtctgaacccagagtcagcacacgagagggctaa

SEQ ID NO: 2 - DNA, 7536 bp, Artificial Sequence
  Synthetic
atggaggacatcgtgattgccggaatttccggaagattgcctgagagcaacaacttggaggagttttggcagaacctcttcaatggcgtggacatggtgacagaggatgaccgaaggtggaaacctggattgtacggattgcctagacggaatgggaagttgaaggagattgaccggtttgacgccgcattttttggcgtgcatcctaaacaagcccatacgatggaccctcaattgcggttgatgttggagatcagttacgaggcgattgtggatggaggaattaatcccgtgagcatgagagggagcaaaactggcgtgtacatcggagtgagcggatctgaagcaggagaggccttttctaaagaccctgaagagttgttgggatactccatgacaggatgtcaacgagccatgttcgccaataggctcagctacttcttcgactttaacggacctagcacggcaatcgacacagcttgttcttctagcttgttggccttggaaaacgccttcaatgccatccggcacggacaatgtgatgccgccttgattggaggagtgaatttgttgttgaagcccaatacctccgtgcaattcatgaagctcgggatgttgtcccctgagggaacctgtaagtcctttgatgcctccggaaatggatactgtcggtctgaagcagctgtggctgtgttgttgacgaaaaagtctatggcaaagcggatctacgccaccgtgttgaatgcggggaacaatacagatggatacaaagagcaaggagtgacctttcctagcggagagatgcaacaacgattggtgcggtccctttaccaagaagccaacatctcccctgagcaagtggagtacattgaggcccatggaaccggaaccaaagtcggagatcctcaagaagtgaatggaatcgtgagcgtgttttgccaatcccctagagatcctttgttgattggatccaccaagtccaatatggggcaccctgaacctgcatctggattagccgcattggccaaagtcgtgttgtcattggagcatggagtgtgggcacctaacgtgcactttaacgagcctaaccctgacatccctgccttgacagatggaagattgtgtgtggtctccaaacctacacctgtgagaggaggaattgtcggaattaattcctttggatttgggggatcgaacgtccacgtcatcttgtctccccatgtgagcgactcttctgacaaaacacctgccccttctgtgccccgattgttgcaagcttctggacgaactgaagaggccgttacagccttgttttctaatgcccaacaacaccaagagaactcctcctacttgtccctgttgaatgatgtgtctggtgtgcctactgctggaatgccttaccgaggatacactttgattggcgcccaaggagaattgaccgaagtccaacagacccaacctacgcctcgacctttgtggtacatctgctccggaatgggaacgcaatgggctggaatgggacaatctttgatgcaattgtctgagtttcgagagtccattaaaagatccgatatcgccctgaaggacaccggattgtgcgtgtctcgattgttgatggaggccgatgagagcacctttgaagacaccgtccatgcatttgtgggattggccgccattcaagtggcccaaatcgacatgttgcggaagatggggttggagcctaatggaattgtgggacattctgtgggagaattggcatgtggatatgctgatggatctttgagccattccgaagccattttggcagcatactggcgaggaagatgcattaaagaggcgaacttgccccctggaggtatggcagcagtgggattgacatgggaagagtgtaaagcgcaatgccctcaaggagtggtgccagcttgccataatgcagaagatacagtgacaatttctggaccccaagactccgtgtccaaatttgtggcccaattgaaagaatctggcgtgtttgcgaaagaggtgcgatctgctggagtggcctttcattcgtactacatggcctccattgcccctgccttgttgtctgccttgcaaaaagtgatcaagtccccccgccctagaacagcacgatggatttctacatctattccccaatccgactgggaatctcctcttgccttgtactcttctgctgagtatcatgtgaacaatcttgtgtcccctgtcctcttccaagaggggttgaatcatgtccccgataatgccgtggtcgtcgaaatcgcccctcatgctttgttgcaagccatcctcaaacggtccttgaagcctacctgttccattttgcccttgatgaaacgtggccatgccaacaacctcgagttctttttgtcccatgtcggaaaagtgtacatgaacgggatcaacgtggattccaacaagctttaccctgccgtgaaataccctgtgcctcgtggaacccctctgatttccccttacatccagtgggatcactcccaatcctgggatgtgcctaaagtggaagactttcctgccggatctggcggatctacatctgcaactgtgtacaatatcgacatgaatcctgaatcccctgactactacatgattggacattgtatcgacggccgagtgttgtatcctgcaacgggctacctggtgttggcttggagaaccttgatgcgctctttgggaacggtgatggaccatacccctgtgacgtttgaagatgtgaccatccatcgagccacgatcttgcctaaaactggatctgtgcaattggaagtgcggttgatgcctgcaacaaatagatttgaagtgtccgagaatggcaatttggctgtgtccgggaaggtgtccgtgttggaagactctgggttggatgcgtttcatgccgagctgaacaagcccattaccgctgacaatgaagaccctaagctccgcctgaaatctggagatatctacaaggagttgcgattgaggggatacgactacggaaagacctttcaaggaattttggagagcaataatgccggagactccgggaaactccactggacgggaaattgggtgacgttcttggacacgatgctgcagatgatcgtcgtgggattgcctggaagaagcttgagactccctacccgaattcgatctgtgtgtgtggatcctaaattgcatgaggaacgcgtgaatgactacgaaggagaccaaaaagccgtgaatgtttttgtgaatcggtgcctcgacaacatcacagccggaggagtgcaaatctgtgggttgcatgctacagccgcccctcgaagacaacaacaacaaacaccccctaccttggaggaattcgtctttgtcccctacgaagatgccgattgcttgagaaccaatgagaaattggccgaccaattgcgacactgtaaaggattggtgcaacgattgcaacgcaaattggccaaacaaggcgtgaagatctccatccctggcttggaaggagcgtccgaagggcaattgattgaagcagaggccgagaaagggttgttgaggttgttgtccgtgttgtgcggcttggagttgaatgggaatttgagatccgaattggaacaaacggtgcaaaaagagagagactgcttgctgcaagatcccctgttgaatggcttgttggattctcaagcgttgcggcattgcctggataccgcgctggaaaattccacacctggcaagttcaaggtcttggaggcccttgctgctgatggacgcgtgttttctcaagctgtgtctttgctgaatattcaacctatgttgcgtttggactacactgcctctgatatttccgccgaccaattgtccgcccaacaatcttctttggaagagcagggaatctctaccgcccagtgggatcctcttcaaggacccgtgacaggaggattggatggagccgatcttgtggtgtgcaattgtgctgttggatctgccacgaatcctgcattgttgattgagaatttgacatctgccgctagagaaggaggattcatcttgttgcacacgttgttgagaggagatacgttgggagaaacagtggcgttcctcacgagccagaataaccggaagggcctgttgacgcaaaccgagtgggaagagctgttccaaaaagcctccctgaatgtggtgatgttgcggaagtcctattacggaagtgccttgtttttgtgtcgaagaagccaacaatccagccaaaagcaacctattcacatcttcgtggaccctaccgactacaaatgggtggagaccttgaaatccacactcgccgagtcctccgatatccccgtctggttgattgccaccaaaggccataatggagtcgtgggaatggtgaactgcttgcgtcaagagcctggaggaaatagaattcgatgcacatttgtctccaatttgtccaaaggagctgcagtgccttccttgttgcctaatgagaaggtgatgaaagccctgttgaagaaggacctggtgatgaacgtccaccgcgacggactgtggggagtgtttcgacatcaattgctgacccaagacctgtccgaagaattgaccgagcaggcctacgtgaatgtgttgacaagaggagatttgtctagtttgagatggattgcgtcccccttgaggcattttgtggcatcttctcctaatgtgcaattgtgcagagtgtactactcctccctgaacttccgcgacatcatgctcgccaccggaaaattgccccctgacgccatccctggagatgtggcattgcagcaatgtatgttgggaatggagttctccggaagagatccttctggaagaagagtgatgggattgttgcctgctaaaggattggccacatgtgtggatgccgataaaagatttttgtgggatgtgccttcttcttggacgttggagcaagccgcctctgtgcctgtcgtctacgctaccgcctactactccttggtggtcagaggaagattgagacctggagaaagtgtgttgattcattccggatctggtggagttggacaagccgctattgcaattgccttgagcatgcgctgtagagtgtttacaacagtgggatctaaagagaagaagcaatacttgcaagagcgcttcccccagttgacagccgagtccttcgccaattcccgcgattcctccttcgagcaacatgtgatgctgaacacacaaggaaaaggagtggacctcgtcttgaattccctcgccgaagagaaattgcaagcctccttgagatgtcttgccagacatggacgatttttggaaatcggaaaatacgatttgtccaacaacacccccctgggcatggccctgttcctgaagaacgtcgcctttcacgggatcttgctcgatgcactgtttgaagagggcaatcgtgaatgggaagaggtgtccgacctgttgaagaaaggaatttcctccggagtggtgcaacctttgagaacgaccgtgtttgagagaaaccaagtggaagaagcatttagatatatggcgcagggaaagcacatcggcaaggtgttgctgcaagtgcgctccgaagaatcctcctcctctggacctgcagtgtctgctttgagtattcctgccatttgccgaacgttttgtcctgcaagtttgtcctacatcatcaccggaggcttgggcggatttggattggaattggcccaatggttgacagaaagaggagcaagaaaattggtgttgacgtctagatccggaatccgcaacggctaccaagctaagagagtccgagagtggcaagccatgggaattcaagtgttggtctccacatccgacgtgtctaccttggatggaacagaacgattgatcacagaggcctgtaggttgggacctgtgggaggaattttccatttggcaatggtgttgaaagatggaatgctcgagaatttgacccctcaggagttcatcgaggtgaatcggcccaagtacgacgggacgatcaatttggacagcgtgacccgacaaaaatgcccccaattgcagcaattcgtcgtgttttcttccgtgtcttgtggacgagggaatgccggacagtccaattacggatttgcaaactccacaatggaaagaatctgcgaacaaagacgacaggagaatttgcctggattggccgtgcaatggggagccatcggagatgtgggagtggtcttggagacaatgggaggcaatgatgccgtgatcggaggaacattacctcaaagaatgtcttcctgcttggaggtgttggaccgctttttgtgtcaacaacgacctgtgatgtcctcttttgtgttggcagaaaaagtggtggtgacaaaaggagagggatccggacaaaaggacctggtggaagctgtggcgcacatcttgggagtgagggacgtgaatagcttgaatgccgacgcatcattggccgatttgggattggatagcttgatgggagtggaagtgagacaaaccttggagagagactacgacattgtgatggctatgagagagatcagacaattgacgatcaacaagttgcgggagttgagcaagcaatccggcgggaaggaggaatcccctgtgaagaggtctggagcccaagcattgttggaaagcgacttgtcccgaatgttggtgaatcctgacggacctacaatggcacccttgaatgaagtccaatccgcagaaagacccttgtttttggtacatcctatcgaaggatctattgccgcatttcgaaccttgacagcgaagctcagcgtgccctgctacggattgcaatgtaccaaagccgcccctttggactctatccaatctttggccgcctactatgtcgaatgtgtgaggcaagtgcagttggaaggaccctacagaattgccggatactcctttggagcttgtgtcgcttttgaaatgtgttcccaattgcaattggcgaaatgccctgtggagtacctgttcctgttcgacggatcccactcttacgtcgccgcgtacactcaatcttatcgagccaagttgacccccggaaaagaagctgaagcagaaacagaagccttgtgtgcctttatccagcagttcaccggaatcgagtacaacaaactcttggagaccttgttgcctttgtctgatttggaagccagagtggacaaagcagtggacctgatcacctcctcccacaagaacgtgtcccgcgatatgttgcattttgccgcctccacgttttaccacaagttgaaagccgccgacagatacgtgcctacatccaaataccacgggaatgtgaccttgttgagagccaaagcatcttctgagtatggagacggattgggatctgactacaaattgcacgaagtctgtgacgggaaggtgtccgtgcatgtgatcgagggcgaccatagaacctttttggagggagaaggagtggagtctatttccagcatcatccacagcagcttgtctgagcctagagtgtccacgagagaaggataa

SEQ ID NO: 3 - protein, 2511 aa, Danio rerio
  misc_feature: Type I Fatty Acid Synthase (FAS)
MEDIVIAGISGRLPESNNLEEFWQNLFNGVDMVTEDDRRWKPGLYGLPRRNGKLKEIDRFDAAFFGVHPKQAHTMDPQLRLMLEISYEAIVDGGINPVSMRGSKTGVYIGVSGSEAGEAFSKDPEELLGYSMTGCQRAMFANRLSYFFDFNGPSTAIDTACSSSLLALENAFNAIRHGQCDAALIGGVNLLLKPNTSVQFMKLGMLSPEGTCKSFDASGNGYCRSEAAVAVLLTKKSMAKRIYATVLNAGNNTDGYKEQGVTFPSGEMQQRLVRSLYQEANISPEQVEYIEAHGTGTKVGDPQEVNGIVSVFCQSPRDPLLIGSTKSNMGHPEPASGLAALAKVVLSLEHGVWAPNVHFNEPNPDIPALTDGRLCVVSKPTPVRGGIVGINSFGFGGSNVHVILSPHVSDSSDKTPAPSVPRLLQASGRTEEAVTALFSNAQQHQENSSYLSLLNDVSGVPTAGMPYRGYTLIGAQGELTEVQQTQPTPRPLWYICSGMGTQWAGMGQSLMQLSEFRESIKRSDIALKDTGLCVSRLLMEADESTFEDTVHAFVGLAAIQVAQIDMLRKMGLEPNGIVGHSVGELACGYADGSLSHSEAILAAYWRGRCIKEANLPPGGMAAVGLTWEECKAQCPQGVVPACHNAEDTVTISGPQDSVSKFVAQLKESGVFAKEVRSAGVAFHSYYMASIAPALLSALQKVIKSPRPRTARWISTSIPQSDWESPLALYSSAEYHVNNLVSPVLFQEGLNHVPDNAVVVEIAPHALLQAILKRSLKPTCSILPLMKRGHANNLEFFLSHVGKVYMNGINVDSNKLYPAVKYPVPRGTPLISPYIQWDHSQSWDVPKVEDFPAGSGGSTSATVYNIDMNPESPDYYMIGHCIDGRVLYPATGYLVLAWRTLMRSLGTVMDHTPVTFEDVTIHRATILPKTGSVQLEVRLMPATNRFEVSENGNLAVSGKVSVLEDSGLDAFHAELNKPITADNEDPKLRLKSGDIYKELRLRGYDYGKTFQGILESNNAGDSGKLHWTGNWVTFLDTMLQMIVVGLPGRSLRLPTRIRSVCVDPKLHEERVNDYEGDQKAVNVFVNRCLDNITAGGVQICGLHATAAPRRQQQQTPPTLEEFVFVPYEDADCLRTNEKLADQLRHCKGLVQRLQRKLAKQGVKISIPGLEGASEGQLIEAEAEKGLLRLLSVLCGLELNGNLRSELEQTVQKERDCLLQDPLLNGLLDSQALRHCLDTALENSTPGKFKVLEALAADGRVFSQAVSLLNIQPMLRLDYTASDISADQLSAQQSSLEEQGISTAQWDPLQGPVTGGLDGADLVVCNCAVGSATNPALLIENLTSAAREGGFILLHTLLRGDTLGETVAFLTSQNNRKGLLTQTEWEELFQKASLNVVMLRKSYYGSALFLCRRSQQSSQKQPIHIFVDPTDYKWVETLKSTLAESSDIPVWLIATKGHNGVVGMVNCLRQEPGGNRIRCTFVSNLSKGAAVPSLLPNEKVMKALLKKDLVMNVHRDGLWGVFRHQLLTQDLSEELTEQAYVNVLTRGDLSSLRWIASPLRHFVASSPNVQLCRVYYSSLNFRDIMLATGKLPPDAIPGDVALQQCMLGMEFSGRDPSGRRVMGLLPAKGLATCVDADKRFLWDVPSSWTLEQAASVPVVYATAYYSLVVRGRLRPGESVLIHSGSGGVGQAAIAIALSMRCRVFTTVGSKEKKQYLQERFPQLTAESFANSRDSSFEQHVMLNTQGKGVDLVLNSLAEEKLQASLRCLARHGRFLEIGKYDLSNNTPLGMALFLKNVAFHGILLDALFEEGNREWEEVSDLLKKGISSGVVQPLRTTVFERNQVEEAFRYMAQGKHIGKVLLQVRSEESSSSGPAVSALSIPAICRTFCPASLSYIITGGLGGFGLELAQWLTERGARKLVLTSRSGIRNGYQAKRVREWQAMGIQVLVSTSDVSTLDGTERLITEACRLGPVGGIFHLAMVLKDGMLENLTPQEFIEVNRPKYDGTINLDSVTRQKCPQLQQFVVFSSVSCGRGNAGQSNYGFANSTMERICEQRRQENLPGLAVQWGAIGDVGVVLETMGGNDAVIGGTLPQRMSSCLEVLDRFLCQQRPVMSSFVLAEKVVVTKGEGSGQKDLVEAVAHILGVRDVNSLNADASLADLGLDSLMGVEVRQTLERDYDIVMAMREIRQLTINKLRELSKQSGGKEESPVKRSGAQALLESDLSRMLVNPDGPTMAPLNEVQSAERPLFLVHPIEGSIAAFRTLTAKLSVPCYGLQCTKAAPLDSIQSLAAYYVECVRQVQLEGPYRIAGYSFGACVAFEMCSQLQLAKCPVEYLFLFDGSHSYVAAYTQSYRAKLTPGKEAEAETEALCAFIQQFTGIEYNKLLETLLPLSDLEARVDKAVDLITSSHKNVSRDMLHFAASTFYHKLKAADRYVPTSKYHGNVTLLRAKASSEYGDGLGSDYKLHEVCDGKVSVHVIEGDHRTFLEGEGVESISSIIHSSLSEPRVSTREG

SEQ ID NO: 4 - DNA, 882 bp, Danio rerio
  misc_feature: PPT gene coding sequence
atggacggggtccggtgggcgttccgctgcggttcgtgggtcccgagccgctccgagtggaccctcgcggctcggtgtgtccagcaggaggaaaaactgcgaatcggacaattcgtgttcgctaaagatgccaaatccgccatggctggacgtctgctcatcagaaagctggtttgtgagaagatgggttttgcatgggacgggtttcgactgcaacgaacagaacggggcaaaccgtacctgccacaaacctcatccgcacccagctggagcttcaacgtgtcccatcagggagattacgccgttctggcagcagaagcaggtcgtcaggtgggcatagacgtgatgaagaccagcagaccaggcagcagctccgtgcaggagtttttccgcatcatgaatcgtcagtttacagatctggagtggacgaacatccggacggcgggatcagactgggaccaactgcacatgttttatagacactgggcattaaaggagagcttcaataaagcgattggcacaggattgggctttgacctgcagagggcggagtttcacatctcacccaatcagatgcgagagggccaggtgtaccggcagacacggatgtacctggacagtgaagaagaggaggactggacatttgaggagagtttattggacaaagaccatcacgtcgctgtggcgctgggaaaaccggatatcagcatgtctaagaaggatggtggaagttcttgtgaagctccgccagctgcatttacagtattgagtttcagtgatctggtgtcacaagccacgcccctactagatgaagaccccacctactgggaggagtttcagaagaaaaaggaagcaccttcaaggcaaagtgaccaggaatga

SEQ ID NO: 5 - DNA, 882 bp, Artificial Sequence
  Synthetic
atggatggagtgagatgggcctttagatgtggatcttgggtgccttccagatctgagtggacattggcagcaagatgtgtgcaacaagaggagaagttgaggatcggacagttcgtgtttgccaaagacgccaaatctgcaatggccggacgattgttgatcaggaagttggtgtgcgagaaaatggggtttgcctgggacggatttaggttgcaaagaacagagcggggaaaaccttacttgccccaaacaagttccgccccttcttggagctttaatgtgagccatcaaggcgattacgcagtgttagcagccgaagctggaagacaagtgggaattgacgtcatgaaaacgtccagacctggaagctcttctgtgcaagagttcttccgcatcatgaatcgccaatttaccgacctcgagtggaccaatatccgaaccgcaggatctgactgggaccaactgcatatgttttaccgccattgggccttgaaagagtccttcaacaaggcaatcggaaccggattgggcttcgacttgcaacgtgccgaatttcatatctcccccaaccaaatgcgagaaggccaagtgtaccgacaaacaagaatgtacctcgactccgaagaagaagaagactggaccttcgaggagtccttgctcgacaaagatcaccatgtggctgtggccttgggaaaacctgacatctctatgtccaagaaagacggaggatcctcctgtgaggcaccacctgcagcttttacagtgttgtccttttccgacttggtgtcccaagcaacacctcttttggacgaagaccctacatattgggaggagtttcagaagaagaaggaggccccttcccgacaatccgatcaagagtaa

SEQ ID NO: 6 - protein, 293 aa, Danio rerio
  misc_feature: pantetheine phosphotransferase (PPT)
MDGVRWAFRCGSWVPSRSEWTLAARCVQQEEKLRIGQFVFAKDAKSAMAGRLLIRKLVCEKMGFAWDGFRLQRTERGKPYLPQTSSAPSWSFNVSHQGDYAVLAAEAGRQVGIDVMKTSRPGSSSVQEFFRIMNRQFTDLEWTNIRTAGSDWDQLHMFYRHWALKESFNKAIGTGLGFDLQRAEFHISPNQMREGQVYRQTRMYLDSEEEEDWTFEESLLDKDHHVAVALGKPDISMSKKDGGSSCEAPPAAFTVLSFSDLVSQATPLLDEDPTYWEEFQKKKEAPSRQSDQE

SEQ ID NO: 7 - DNA, 12447 bp, Artificial Sequence
  Synthetic
atgtccgaccagcagcaacaacagcagatcaaggtggaggagacagcccctgtggcccaagagcctaaaacccacttgtttgtggaccctgaagtgtcctcctgtattgccgcctttggaggccaaggatccgattggttgggagagttgcgatccttgcacaagaacgggaaatcctccgtgcgggagtttttggaactgggcctgtccaaattggaggatattgccgcgtccgatgagtggtacgccaaccacgggggcttgaatgtgagagcctggattgcctccgacaaatctgtgccgagcttcgacctgttgcgctacgcccctgtgagctttccgctgatcttcatgacgcaaatggccaactacatgcgggtgctggagttgttgggaacgagccacgagaaagtcgcacaacaaggctggtttaagggagccttgggacacagccagggagtcgtcgccgccgctgtgactgctgctgcatctactgatcgagaattgagaaacttgtccgtcgccggtttggagttcatgtcccaaattggattgggagcccaaaagagcatgaacttcgagttgtcccgcagatccgccggacctgaatcccctatgttgtctgtgcaaggcatgtccgaagccacgctcctgaaggccttcaaggaggccacgaagctcgccgtgcagaaagagaccatgatggccaaattttccacgtcctccaaggacgacaaagccgcccctaatgcctcccaaagattgggaattgccttgtgtaatggaaccgacgactacgtggtgtgtggagaacctaaggacctgcgcatgttgcggaaggtgatcgtgtccatgtccgccgaagtcgggaaagaggcccaagcccgagtgccattcagcaaaagaaaacctgtgacccaaacgacgtttctgcgcatgacagcgccattccattctgccttgaatgccgaagcctttgaacaagtggccgcttgggcagcatcttctgcttttggacaggaattggcccaacgcaccttgagaatccctgtgtgggacaccgagaaaggagccgacctccgcaagatggagccttcccaagtggtgaacatgttggcccgaaataccttggtctcctccgccaatttgctcagcacattgagagccgccgaagctgactgtaaagcctcccacttgatctcctttggacctggatctgtggccggacaccttatggccaatgcccttgtgggaactggaatccaggtgatccaggcgaatgaccctgactccaagagcaaggaaaatgccgccaccgcgagcagctcgtcgagccggagcagcgccaaatctcttgccgccatcttgacagccgaaaaacctgagcgaatccctgttgcaacaccttggggagagaaattcgcccctaagattgccgtgcgtgcgtgtgatggagaacgagtgttgatgaccaagtacacctccaccatcggacgagcccctgtgatgatgtccgggatgacgcctacgacgtcgttccacggaattgacttggtggccgcctgtgcaaatgccggatacaatgccgaattggctgctggaggattgcctacacctgacctgtttaagatgaaggtgcaggaactggcctccaagttgaaccctggagtgggaatggccattaacatgctgtacctgaacgcctaccagtggggcttccagttcccactggtgtgcgaattggcaaaagccgggctccccatcgagagcatcacaatcggagccggagtgcctaccgaagaaaaagccaaggacatctttgacggattgcaaggagccggcatcaacttgatcgggttcaaacctgggtccaaacaagccattagagacgtgctccaacttgcctccttgagaccctccatgaacatcatgctccagtggacctccggacgcggcggcggacatcacagctttgaggacttccacgagcccttgttggccacatacgaagagatcagacaacacgacaacatcatcctggtgatcggaagcggctttggagacgcccaaggagtgatgacataccttgatggatcctggtcccaatcccccaaatttggacgcttggccaaaatgcctgtggacggcgtgctcttcgggtcccggtgtatggtggcgaaagaggcagccacagcccctgaagtgaaacaattgatcgtcgatgctgctggattggaagacgagttgagctgggagaagtcctacgacgaagtggcaggaggagtcgttactgtgcaatctgagttgggagaacccattcacaaattggccacacggggcatactgttgtggcgggagttcgacaagcggttcttcagcttgcctcggggggagaaacggagagatgccatcttggcagcaaaagacgagattatcgcgaagctcaatgcggacttccagaaaccctacttcggccgcaaacgagacggatctgtgtgtgaagtggaggacatgacatatgctgaagtgctggagcggatggtgcagctgatgcacatcaagaacgggggggacaaggccgggcggttggcgcctacacgatggattgatcctacatatgcctctagagtgatgctgatgatgcaacgttccgccgcgagattggccaaggacaaaatggacaaggtggtgccttccaacaagctgttgatggaagaccctgacaaagccatcgccgacttcttggcagccattcctgccttggaggactccctgatggccgacgacgacgtgacctacttcctcgacttgtgcaaagtgcctacacgtggaaaacctgtgaacttcgtgcccgtggtggatgaggacttggtgttttgggtgaagaaggactccctgtggttttccgagcagttggatgcagtgcctgacagagatcctggacgagtgtgcgtgttgcatggacctgtggctgcccgatactccaccatcgtggatgagcctattgccgacattatggggaacatccacaaggacttggtgcgggacctgaagtgtgacgaggtgaaagtgaacgtgttggcccctgtggagttgcaacaagcaagcgtgctgaagatcatctccgagacaccccaattggtgagaggacgctcctttgtgcccaaccccatcgccaaggtgttgaaacgcgaggccttcgagaaagtgaagtacacatccgagggaggattggagtccatcaacgtgcaagaccctgagcgaggattgaccgtggccaccttgtcccaagtcggatccgacaacaaattggcagagttgcaagtgttcgacaaagaagccggagccgtgttgaagcagaagttcaccatcgacctgaccagcctccgccccatctttcagacagaggaggacaacttgagcgcaacgaagcagttgtacagaaccgcctgggactgtcaaggcgagttccatgccggagacacgttcaccgacgaagtggtggtcacatccgaaaacattgaggcctttaatcgagggacgcataccgagtacaatggatccgcagaagcccctatcgacatcagcatcatggccggatggcggcctttggcccgagcgttgttcgtggaagaattgcaatccaacctcctgaagctcgtccatctgaccaacggcatccggttgcccaaccccaaaacccgaacacctgtgaaagccggagaagtgattagatctgaggtgcgcattacaggaatcacgatccagcccaaagtgggcaagaaagtggcggtgaagggcatcatcacccgcgcctccgacaaaaatgccacgcccgaaatgtgggtcgaaatgaattccgccttcctcatccgcggagtcgccgagacacctgaagaatacgccacgacgttcgaagagtttcctgccgaaacccatgtgattgatgtgaaagatgccacggtggccgaattggtggcctctagagcctggatcaaactggacaacggccgcaaggtgcaagagggagaccgagtgacgatttccttgtcccatgtgtccaatcgctttgccggacccaaccgcttgaaggacatcaaggtgaccggagacgtgtttatcgagtccacctccgtgaaatcctccaattccggatccacccctttgggatcccctacgaattcctccgtctctggaacacaatccgacgacttcgtggacgtggacacctcctccaaggtgaaagcaggaacagtcgagtttgcctcctccgagggcgaagagttccagttgaatcccgtgatgtcgttcctggaaaagtactccgagcctatgcacaatggccatatcgccgagaatggcgggtacgagttgatcgccgagcctatggtggtgcctgcacctgctgattgtacgatgtacgcacgcggatccagagatgccaatcctatccatcgcgaatccgcattcgccgtgttggccgatttgcctggaggagagcctatcgtccatggaatgtggacagcatgtatggctagagctagattggaagaaggacctgccggaggaaatcctgcccgaatcgtctcctacgaggcctcctttgtggatatggtgcattgcggcgacgaactggtggtgacggctaaacaaacaggggtgaaggacggactgatgctgatcaacctgagcgtgaatcgggcctccgaccgagcattggtgatgactgctagagctgaattggaacaacctacaacggcctacttgttcacggggcaaggatccgcctctcccggaatgggaatggacagatatgcagcttccgcaacggtgcgcaaagtgtgggatgtggcagatgagtacttgagaaaccgatacgggttcagcatcctccagatcgtgcgggagaaccccaagtccttgacggtccactttggaggacctagaggaaaggtgattcgcgaaaatttgcgctccctgcagaccgaggaccctacaaccgggaaaaaaatgcccctgatccccgagatctcctccacgaccaagtccttcaccttcaattcccctaccggcttgttgtttgccacccaattctcccaacccgccctcgtcttggtgcagaaagccgcctttgaagaattgagagaagccggccttgtccctgaaaaagccttctttgccggccactctttgggcgagtatgccgcattggccggatttgccgatgtcctgtctgtggaggaccttgtggaaaccgtgtttttgaggggaatggtgatgcagaatgccgtgcctagggatgccgacggaaccagcaattacgccatggtggcagcaaatcctttgagagtcggacgtggatttacccctgagaccttgggagaagtcgtggacttgctgtgtgagcgggaggacttgggaaagcccttgctgcagatcgtgaactacaacgtgagatacacccagtacgtggtggcaggagagttgctcgcattggacgccttgggagaagccctgaatttggcctttgcaaccggaaataggaatgccgcggaattggccgagaaaggagcacaagcagccttggcctctttggcaaagaggggaggacggaaagagcccttgaaacggggaaaagccacaattcctttgcctggaatcgatgtgcccttccattcccggaagttgttgcccggagtgggagcgttcagacaactgttgagccctagacttgccttgagtacgatggagcggatctaccaccggttggtgggcaactacatccctaacgtgacagccgaggtcttgacgttggacagatcctacgccgagaaagtgcagaaagtgaccggaagtgcccctatgttggagttgttggccgactacgattccgccacccctgccgaaaaatgccgaacgttggtgatcgagttgttggcacatcagttcgccatgcctgtgcgctggattgagacccaagatctgatgtttggagcccgagtgcaacgcgtgatcgagatgggacctgcagccacattgacaacaatggccaagcacacgttgaaatccggagcctttggagacgccgacgagtacaaccctgagatcatgtggtggaagaacgaccgagagcgagtgtactacgagttggacgacgagggaccgagcttcgccgcctttgtggaacaattgaaggccgagatggagtccgaggccggagactctgaggagggagaagtgtccgaagcacctgcagcacctgcacctacacctgctcctgcacctgcacctgtggcacctaaacctgcacctgcacctgttgctgctcctgcacctgcaccttctggaggagcatctacacctgatgcacctgttgatacaaaacatgttttgagagtgttgttagcctccaagctcaagaagcccattggcgaagtccctgcctctacgtccgtccaaaccttgtccgccggccgatctgctgtccagaacgagatcatgggcgagttgtccgccgagttcaaaggcggcatccccgacaatgccggagaaatgcctttgtccgaattggccggaaatttgtcctcctacaaagaacctggagccgtgtctacaaaacttgtcacccgaacattgtctgcagccttgcctggaggctttggagccaatgccgccaaggactacctcacgcaacactgggggttgggaacgggacggaacttctccgtgctgttgcactcctgcacaatggcccctgagaaacgcttgaaatctgaggaggaaggaaaacaatggctcgactccgtctgcaaagcctacggtgatgatgttggagtgtctttgtcccctgccggagctggaggagctggaggagcaccttctggaatgatgatgatgccccagatgatgtccatgggaggacctgccgccgtgccgccaccggatgcacctgtctctgcattgcatgctatgagagtgatgttggccacaaaattcgagaaagggttcaacgagattagcgattctgccacagtggcctctctgtccaacgggaaatccgccctccagaacgaggtggccggagatttggcagccgaatttggagctgaaggagacgattccgcccagaagcccctcaccgaactcgccgccgcttttcaagccggatactctggacctggtaaagttttgtcaagagacatcaataaagtgttgggacaatgtctccccggaggatttggactgtccgccgcacgagcctaccttgcatctgaccgccttttgcctgccggacgagttgaatccgtgatgatccactccctgaccatggcccctaaggagcgcatcaagtccgccgaagacgcaaaggcctggctggatacagtgtgtggagcatatggatcttttgcaggaattgatatccctagagctggagcaggaggaggaggaggagccatgatgggatttgcgggacctggagtgtcttctgccgaagtgaatgggttgaaagcgaacctgcagtcgatggtggagacccagttggaggcccttcagcggttcatggagcaggaccccttgcatgccgacagattgttggatgtggagagaaaattgagaggagaaacagaggccaagttggacgccatccacgccgagttgacagtggacttctgcgagagagtgcaaccccaattcgacgagaaacgtgtgcgagtgtacgactccttctggaactgggtggtgcaagacgcgatgcagatgcacctgcacgtgctgtcccggttgaatgaggcccggaaaggacaatctacaggattacctgcaggagatgccaatccccatttcgaggacatgtccaagtggttgttgggaacgtcgagcagcgaagtgccccctacagcctggtttcgcaatttcttgtgcaatagagccacgccccagttgttgcaagccgtcaagttcttcgccaactccatgcacgaggccggacacgtggactacgcccaagccattgctttgcttgccgaacaggtgcaatgctggttgaataatgtgcctgtacatgtcgccacctttgaccccgtctcccccaatgtgcgagtgttggacaatggaacagtcgactacttcgagacccctagagaaggcgtgcctgacgccgtgagatacgtggcagaaatgagccgcggcctgttctacgtgagaagatctcctgccagagtggccaatccttcccaagcagtgaatgtggctggagatggacaattggcattgcctcctgctgctgattctaccggattgcaacctgccgatggagaacttgcttctggatggagacgacctagatccgaggccgaattggcccgagagttgaacaaccggtccggatccggccttgaagcattggacttggaagccgatgaagcctctgaggagtctaaagaagccttgcccgaaggacctaccttggaccgattgagattgacggtgtctagagatgcagcctcttctggagaagatgccggaagccctgggaaaatttccacgagctcgctgaagaacggctacgagtccatccacgtgtccaagcaggtgcccttcgtccacctcaagtccttgtccggagtcgacaaatctgtgcgcatcctcaatgagcagcttacctccgagtacttttcgtgccttgacgaaatcgccacatccggagtgtcctttgccggccaagtcgccttggttacaggagcaggagcaggatccattggaacagagttggtgaagtccctgttggagggaggagcaaccgtgttgtgtgccatgcgaaccgccagatccgagaatgcattgaccaaggaatacgcccgctttcagaacatctacaaggagtttggggccaaagacagcaagctctacctcgtgccgtgcaactgtgcgtccagccaggacatgaagagcatcgtggcctacacgtacgagcaattgggattggacgtggacttcgtcgtgccctttgccgcagccgcccaacaaggaaaagatatctcctctattgacgccgcctctgaagcctctcataggatgatgatgaccaatgtggtgcgcctcttgggagccttgcgtgatgccaaagccagccggggaattgtgacaagacctgccatggtgttgatcccttgctctcctaatcatggcgagttcgggaatgacggcctgtacgccgagtccaaattgggatgtgaagccttgctcaacaagtggtcctccgagggatggggagattatttgtccttggccgcctgcgtgatcggatggacaagaagtgccctcatggagcacaacaacattgtcgcccccggaatcgaagccttgggatgcagaacatttgcccctgaagaaacgaacttcaacctggtcggcctcttgcatccccgaatggtgaccttggccgccgaagaacctttgtgggccgatttgacaggaaattgggtggtgatccccaacatgaaggacgcagccgacggattgagaagcgagttgatgaccaagtcccgcattgccagagctgtcgccacatccaaccaattggaagagtccaaaaaacctgagggaggacgggagttgccgccaccggaatccgccggacctttggccggaacgatgttgggaatgacgccgtttccgaccttgcctagcgaagaagcccgaaagtccctgtctgccttggaaggaatggtggacttgcggaaagtcgtcgtgatcaccggatacggagaagtgggaccttggggaaatgcaagaacacgatgggaaatggagagctacggagagttctccctggagggggccatcgagttggcctggatggtcggattgatcaaaagacatgacggacctttgcctagcggacctcctcgacaacggtatgtgggatgggtggatgcaacgagtggagaagcagtggcagatcatgaggtgaaacggagatacgagaagaccctgttgcaatcttgcggaatccggattgtggaacccgccatcttcgagggctacaaccccgacgccaagcgctttttgcatagtgtggtgcttgaccgagatatgcccgccatcgagttggcctctctggaagaagggctgcagtacatgaaggagttgggagaggagtgctgtgacgtgtttgccagacctagcgatggacaatacatgatgcgcgtgaaaaaaggagccgaggtgagcatcgccaaagccctcaagttcaaccggaacgtggccggacaagtgcctacaggatgggatgccaggagattgggattgcctgccgatatcgcgaacagtgtggaccctgtgacgttgtacacgttggtgagcaccgtggaagccttgatggcagctggattgtcagacccttacgaattgtaccaatacgtccatgtgagcgaggtggggaatacaagcggaggaggaatggggggaatgaggtcgttgaagcggatgttccatcagcggaagctcgacgaggacatccctagtgacaccttggccgagtccttcatcaacacgatgcctgcctgggtgaacatgctgctcgtgtctagctccggacctatcaaaacacctgtgggagcatgcgcaacagcagccgaatctttggatattggaatggagaccatcctcagcggaaaagccagagtggtgatcgccggaggatacgacgactttggggaggagggatcctacgagttcgcccaaatgggagccacgaacaacaccgtcttggatgccgcacgtggaagaacagtgagagaatctagccgacctatgtcgtcgagcagggcaggatttgtggaatctcatggagccggaattcaagtgttgatggatgccgaattggccctggagatgggagcccctatcttcgccatcctggccctgacaaataccgccaccgacaaacagggacgctccattcctgcacctggacgtggaatcttgacatctgccagagaaagcaccaaagccggagtgagccctatgctttccttggaacgacgaagacaaggcttggaaatggaattggacgccttgaagacgctgaatgcccagaaggagcagtccgagggggaggatgccgcctttcttgagagactcgtgcagaaacgacgagcagcagccttggagacctggggacaagggtttttcaagaatgacccctccatcgcccctttgagaggagccttggccgtgtggggattgggagtggatgatttgggagttgcatcatttcatggaacgtctacgaagttgaacgacacgaacgagtccggcgtgctgaacaagcagatggagcatttgggacggagcaagggcaacgtgctcttcgtggtggcccagaagtacttgacgggacaccctaaaggagccgcctgtgcgtggatggtgaacgggttggtgcagtgtatgttggacgcacgagtgcctggaaatagaaatttggacaatgtggatgtgaagttgcagacgaactcctacctcgtgtaccccaatgagcccgtgcaactccccaagatcgaagccgccctgctgaagtccttcggattcggacaagccggagcagaggtcgtgatcgtgcatcctgaccgcttgttggccaccttgtctcctgaagccttcgccaactacatcgaagccagaaatattagagaaagacgaacgttccgaaatgcccagaacgtgatgtccggatcccgcaacatggtgatcgtgaaggagcaccccccctaccctgccgagttggaagaagccgtgtacctcgaccctcttgcccgtgcctcctatgatgccaaggagaatacctggacgttccgatccgccgctggacttacatcttctggaatgcctaaaattgcgtcgagccaagcgccgtcgtcgccgaaacctgcagcagcacctgtgtctgaatctgcctcccaatccgcccagatgtccgccaaagaccgattgcagatgacgatggccgagcaagccgccggaatcgcagcaagagctggaggttctggtgtgggagtcggagttgatgtcgaaaatgtgtccacttttgctgattacgctggatccaagcaagacttcatccaacggaacttcaccgaagccgagatcgcctactgcaagtccgccgccgatcctgccgcctcctttgctggaagatgggcagccaaagaagccgtcgtcaaagccttgtcctccattgcccctgattcccgatccttgtgggccggaggtcatgcatctttagttgatatcgaagtggtcgcaaatccttctggagcacctcaaattagattgcacggtcaccctgagcaagtctcccagatgctcgccgtgaacgacctgagcgtctccatctcccataccgccgaagtggcaatcgccaacgccatcgcccgaaagtga

SEQ ID NO: 8 - protein, 4148 aa, Aurantiochytrium sp.
  misc_feature: Type I Fatty Acid Synthase (FAS)
MSDQQQQQQIKVEETAPVAQEPKTHLFVDPEVSSCIAAFGGQGSDWLGELRSLHKNGKSSVREFLELGLSKLEDIAASDEWYANHGGLNVRAWIASDKSVPSFDLLRYAPVSFPLIFMTQMANYMRVLELLGTSHEKVAQQGWFKGALGHSQGVVAAAVTAAASTDRELRNLSVAGLEFMSQIGLGAQKSMNFELSRRSAGPESPMLSVQGMSEATLLKAFKEATKLAVQKETMMAKFSTSSKDDKAAPNASQRLGIALCNGTDDYVVCGEPKDLRMLRKVIVSMSAEVGKEAQARVPFSKRKPVTQTTFLRMTAPFHSALNAEAFEQVAAWAASSAFGQELAQRTLRIPVWDTEKGADLRKMEPSQVVNMLARNTLVSSANLLSTLRAAEADCKASHLISFGPGSVAGHLMANALVGTGIQVIQANDPDSKSKENAATASSSSSRSSAKSLAAILTAEKPERIPVATPWGEKFAPKIAVRACDGERVLMTKYTSTIGRAPVMMSGMTPTTSFHGIDLVAACANAGYNAELAAGGLPTPDLFKMKVQELASKLNPGVGMAINMLYLNAYQWGFQFPLVCELAKAGLPIESITIGAGVPTEEKAKDIFDGLQGAGINLIGFKPGSKQAIRDVLQLASLRPSMNIMLQWTSGRGGGHHSFEDFHEPLLATYEEIRQHDNIILVIGSGFGDAQGVMTYLDGSWSQSPKFGRLAKMPVDGVLFGSRCMVAKEAATAPEVKQLIVDAAGLEDELSWEKSYDEVAGGVVTVQSELGEPIHKLATRGILLWREFDKRFFSLPRGEKRRDAILAAKDEIIAKLNADFQKPYFGRKRDGSVCEVEDMTYAEVLERMVQLMHIKNGGDKAGRLAPTRWIDPTYASRVMLMMQRSAARLAKDKMDKVVPSNKLLMEDPDKAIADFLAAIPALEDSLMADDDVTYFLDLCKVPTRGKPVNFVPVVDEDLVFWVKKDSLWFSEQLDAVPDRDPGRVCVLHGPVAARYSTIVDEPIADIMGNIHKDLVRDLKCDEVKVNVLAPVELQQASVLKIISETPQLVRGRSFVPNPIAKVLKREAFEKVKYTSEGGLESINVQDPERGLTVATLSQVGSDNKLAELQVFDKEAGAVLKQKFTIDLTSLRPIFQTEEDNLSATKQLYRTAWDCQGEFHAGDTFTDEVVVTSENIEAFNRGTHTEYNGSAEAPIDISIMAGWRPLARALFVEELQSNLLKLVHLTNGIRLPNPKTRTPVKAGEVIRSEVRITGITIQPKVGKKVAVKGIITRASDKNATPEMWVEMNSAFLIRGVAETPEEYATTFEEFPAETHVIDVKDATVAELVASRAWIKLDNGRKVQEGDRVTISLSHVSNRFAGPNRLKDIKVTGDVFIESTSVKSSNSGSTPLGSPTNSSVSGTQSDDFVDVDTSSKVKAGTVEFASSEGEEFQLNPVMSFLEKYSEPMHNGHIAENGGYELIAEPMVVPAPADCTMYARGSRDANPIHRESAFAVLADLPGGEPIVHGMWTACMARARLEEGPAGGNPARIVSYEASFVDMVHCGDELVVTAKQTGVKDGLMLINLSVNRASDRALVMTARAELEQPTTAYLFTGQGSASPGMGMDRYAASATVRKVWDVADEYLRNRYGFSILQIVRENPKSLTVHFGGPRGKVIRENLRSLQTEDPTTGKKMPLIPEISSTTKSFTFNSPTGLLFATQFSQPALVLVQKAAFEELREAGLVPEKAFFAGHSLGEYAALAGFADVLSVEDLVETVFLRGMVMQNAVPRDADGTSNYAMVAANPLRVGRGFTPETLGEVVDLLCEREDLGKPLLQIVNYNVRYTQYVVAGELLALDALGEALNLAFATGNRNAAELAEKGAQAALASLAKRGGRKEPLKRGKATIPLPGIDVPFHSRKLLPGVGAFRQLLSPRLALSTMERIYHRLVGNYIPNVTAEVLTLDRSYAEKVQKVTGSAPMLELLADYDSATPAEKCRTLVIELLAHQFAMPVRWIETQDLMFGARVQRVIEMGPAATLTTMAKHTLKSGAFGDADEYNPEIMWWKNDRERVYYELDDEGPSFAAFVEQLKAEMESEAGDSEEGEVSEAPAAPAPTPAPAPAPVAPKPAPAPVAAPAPAPSGGASTPDAPVDTKHVLRVLLASKLKKPIGEVPASTSVQTLSAGRSAVQNEIMGELSAEFKGGIPDNAGEMPLSELAGNLSSYKEPGAVSTKLVTRTLSAALPGGFGANAAKDYLTQHWGLGTGRNFSVLLHSCTMAPEKRLKSEEEGKQWLDSVCKAYGDDVGVSLSPAGAGGAGGAPSGMMMMPQMMSMGGPAAVPPPDAPVSALHAMRVMLATKFEKGFNEISDSATVASLSNGKSALQNEVAGDLAAEFGAEGDDSAQKPLTELAAAFQAGYSGPGKVLSRDINKVLGQCLPGGFGLSAARAYLASDRLLPAGRVESVMIHSLTMAPKERIKSAEDAKAWLDTVCGAYGSFAGIDIPRAGAGGGGGAMMGFAGPGVSSAEVNGLKANLQSMVETQLEALQRFMEQDPLHADRLLDVERKLRGETEAKLDAIHAELTVDFCERVQPQFDEKRVRVYDSFWNWVVQDAMQMHLHVLSRLNEARKGQSTGLPAGDANPHFEDMSKWLLGTSSSEVPPTAWFRNFLCNRATPQLLQAVKFFANSMHEAGHVDYAQAIALLAEQVQCWLNNVPVHVATFDPVSPNVRVLDNGTVDYFETPREGVPDAVRYVAEMSRGLFYVRRSPARVANPSQAVNVAGDGQLALPPAADSTGLQPADGELASGWRRPRSEAELARELNNRSGSGLEALDLEADEASEESKEALPEGPTLDRLRLTVSRDAASSGEDAGSPGKISTSSLKNGYESIHVSKQVPFVHLKSLSGVDKSVRILNEQLTSEYFSCLDEIATSGVSFAGQVALVTGAGAGSIGTELVKSLLEGGATVLCAMRTARSENALTKEYARFQNIYKEFGAKDSKLYLVPCNCASSQDMKSIVAYTYEQLGLDVDFVVPFAAAAQQGKDISSIDAASEASHRMMMTNVVRLLGALRDAKASRGIVTRPAMVLIPCSPNHGEFGNDGLYAESKLGCEALLNKWSSEGWGDYLSLAACVIGWTRSALMEHNNIVAPGIEALGCRTFAPEETNFNLVGLLHPRMVTLAAEEPLWADLTGNWVVIPNMKDAADGLRSELMTKSRIARAVATSNQLEESKKPEGGRELPPPESAGPLAGTMLGMTPFPTLPSEEARKSLSALEGMVDLRKVVVITGYGEVGPWGNARTRWEMESYGEFSLEGAIELAWMVGLIKRHDGPLPSGPPRQRYVGWVDATSGEAVADHEVKRRYEKTLLQSCGIRIVEPAIFEGYNPDAKRFLHSVVLDRDMPAIELASLEEGLQYMKELGEECCDVFARPSDGQYMMRVKKGAEVSIAKALKFNRNVAGQVPTGWDARRLGLPADIANSVDPVTLYTLVSTVEALMAAGLSDPYELYQYVHVSEVGNTSGGGMGGMRSLKRMFHQRKLDEDIPSDTLAESFINTMPAWVNMLLVSSSGPIKTPVGACATAAESLDIGMETILSGKARVVIAGGYDDFGEEGSYEFAQMGATNNTVLDAARGRTVRESSRPMSSSRAGFVESHGAGIQVLMDAELALEMGAPIFAILALTNTATDKQGRSIPAPGRGILTSARESTKAGVSPMLSLERRRQGLEMELDALKTLNAQKEQSEGEDAAFLERLVQKRRAAALETWGQGFFKNDPSIAPLRGALAVWGLGVDDLGVASFHGTSTKLNDTNESGVLNKQMEHLGRSKGNVLFVVAQKYLTGHPKGAACAWMVNGLVQCMLDARVPGNRNLDNVDVKLQTNSYLVYPNEPVQLPKIEAALLKSFGFGQAGAEVVIVHPDRLLATLSPEAFANYIEARNIRERRTFRNAQNVMSGSRNMVIVKEHPPYPAELEEAVYLDPLARASYDAKENTWTFRSAAGLTSSGMPKIASSQAPSSPKPAAAPVSESASQSAQMSAKDRLQMTMAEQAAGIAARAGGSGVGVGVDVENVSTFADYAGSKQDFIQRNFTEAEIAYCKSAADPAASFAGRWAAKEAVVKALSSIAPDSRSLWAGGHASLVDIEVVANPSGAPQIRLHGHPEQVSQMLAVNDLSVSISHTAEVAIANAIARK

SEQ ID NO: 9 - DNA, 1000 bp, Nannochloropsis gaditana
  misc_feature: RPL7 promoter
gaatgctggcgacctcatgaactttgttgatttttttagaattgtgtcatcgaaaaatatacaatgtcgaagtaaagtttatacactctgggtactcttcttctttgccatccagtaggtgtgggataggagtgcgtggtaattctctgagacagttttcctaccatgtttcgggtccacatagtacaagttgttactcgtgacacctcatttcgcttttgtttctccactaatggactacatttttcaaagcagcagcgtccgcgtttctcgattaagaatctcccgtccgctttgagtttgtttattttaaggattagaaggataaaagcggagctgcaaaggtgaaacatgcctgagaggactagaagacatacggagctgaagtgcggggaggttaaacccgatgatcccattccttgcatttgtcaaggctcaccgcattcatagttctaaaaagaaaatatttccgaataatgttaacacaaaaataattaaatactgttctatgtactccgttcatttgttgatggaatcaaaaggccagaaaaaaagagggggggactccaagctgccgcagacgctcgcctcgtctctcgttcgcggaatgccggtcgtggaaccgctgtgaagataccgtgccttggggatcaattcacacattgacgagggcgcctcacgccgtggcgtacttattcgggccaaaatgttatttccaaggggtgacggatggcatgctcgactcgtgtgatggctgatgcatcaaccctttcaatgctccttcctacttcctcttatccactggtagtctgtcatgagatgtggtgaaccgcgtccgtgataatagtgttctcatttaacctaacaaccaacacaatacaaaacttgattagatacccagtgcatcgaattttgggtgccttcaccgacaagcttgcactttctcgagggacgacaacaacaaaccacctacagcattaccgggcaatagcagcacaac

SEQ ID NO: 10 - DNA, 317 bp, Nannochloropsis gaditana
  misc_feature: Bidirectional terminator 2
agtgatgcggcctttaggaaacaccacaaaagtaattgacaatctcaggaacgatctgcgtgtttacagcttcccaaataacaattataccacgtaccaaaaggggtttaatgtatctcacaaattcttctaataggtacagcttctcaaattgggtgtatgatgtgacacttcgtctcacacacgtcacgataattcagcgtatggcttcccttcatcacattcacgcaaacttctacacaaccctgggcatatttcttgtgttggcaacactcccgaaatcgattctgcacacaatggttcattcaatgattcaa

SEQ ID NO: 11 - DNA, 822 bp, Nannochloropsis gaditana
  misc_feature: 4A-III promoter
ggcataaaggacggcaaggaaagaaaagaaagaaagaaaaggacacttatagcatagtttgaagttataagtagtcgcaatctgtgtgcagccgacagatgctttttttttccgtttggcaggaggtgtagggatgtcgaagaccagtccagctagtatctatcctacaagtcaatcatgctgcgacaaaaatttctcgcacgaggcctctcgataaacaaaactttaaaagcacacttcattgtcatgcagagtaataactcttccgcgtcgatcaatttatcaatctctatcatttccgcccctttccttgcatagagcaagaaaagcgacccggatgaggataacatgtcctgcgccagtagtgtggcattgcctgtctctcatttacacgtactgaaagcataatgcacgcgcataccaatatttttcgtgtacggagatgaagagacgcgacacgtaagatcacgagaaggcgagcacggttgccaatggcagacgcgctagtctccattatcgcgttgttcggtagcttgctgcatgtcttcagtggcactatatccactctgcctcgtcttctacacgagggccacatcggtgcaagttcgaaaaatcatatctcaatcttcagatcctttccagaaacggtgctcaggcgggaaagtgaaggttttctactctagtggctaccccaattctctccgactgtcgcagacggtccttcgttgcgcacgcaccgcgcactacctctgaaattcgacaaccgaagttcaattttacatctaacttctttcccattctctcaccaaaagcctagcttac

SEQ ID NO: 12 - DNA, 175 bp, Nannochloropsis gaditana
gaggggggctcgagagggagaggggtgtgtcatatgagggcggcggggaattcgtgtgtgaaaggaaagcatgagtcgataaaggggggggtggagcgagacaaagaagaaacgggggtcgatagcgctgatccaaagcgtcgtctcagctttcttttctcgcgacgccaacctc

SEQ ID NO: 13 - DNA, 399 bp, Artificial Sequence
  Synthetic
atggccaagcctttatcccaagaggaatccacgctgatcgaacgtgcaactgcgaccatcaacagcatacctattagcgaggactactcggtggccagtgcagccctctcgtccgacggtcggatctttaccggcgtgaatgtatatcatttcaccggagggccatgcgcggagctcgtggtcctcggaacggccgctgcggctgctgccggaaatctgacgtgcatagtggccatcgggaacgaaaaccgcggcattctgtctccgtgcgggcgatgtcggcaggtgctgcttgacttgcacccggggatcaaggcaattgtcaaagattccgatgggcagcccacagcggttggcatcagggagttgcttccctctggctacgtctgggagggttga

SEQ ID NO: 14 - DNA, 999 bp, Nannochloropsis gaditana
  misc_feature: TCTP promoter
cgtgcaggtgtacagattgaaggaaacaatggagatatctttggcagttgaaaaccgtgttcgaatcatgcttttctactctccaactgagacgaaatttatagcgccatgtcgcttctgactaccaggcttaggaaggcctcatcacaagctggatcggttcgaattaagcaggcactgaagccaagcttgcaagacagccaccttttaattccctcaaaacactttctcaattcagcccggtaaatatgccgattcacagcggccaagatagaggggaggttagcaagaatgttgcgatccctccccagtcgttgcctcgcacacaacctaggccttcacctttccatggaaaattgagaagtgaatattggttttcttacggcatatcagatgaaatcatgacccctaaacatgaagagctgcaggcaaaacacctgctctggacgagcacgatgaaatctcgagaacccgccgtacttcagttgatcccgcatgatgacggccgccattgaaataagccacctcactttattctagcaccgatttccaccgttgtgagggccgaacgaggacaatttcgtgcgaaacaagcacgaacacgcacacgattagtagtacagacgagcagatcgatggcatgcggcacggtctcgcgttctcggcgaccaggacaacggagcagagggaggcctgccgagttccgaggggcattttagtccaaaattgtgttgacacgtgaacaagtggcttgaaaagaggaaggaaatgcctgggtttcccttcgagagcgggaactcgcttgtgcgtcatcctagctacccatggtccctttgtgggggaggctgtttcgtcctaccgaatgtgtggcgctccatgcatcttctgcctcccaaaccaccaacatgagcacgcgaaggaaggagaaaaaagtggccgcaacgttctcttctcatatttattgtctcatcacaaacataggtacataatacaacaatc

SEQ ID NO: 15 - DNA, 318 bp, Nannochloropsis gaditana
  misc_feature: EIF3 terminator
ggcactgtaaccccggttccgctcgacgaaggctgggagcgccctttcggtgggataaaatggatgctttaccgctgcgcttcggctgaggaagagagaaatgcgagcggggatcggggtcctagaaacgaagaaaggagaacaagttcctggccaaagaaaaacaagacaaataccctctccaggcctgggcccattacttttttttgctgtttcttatacctgcactcgtgcttctctagtctgtcgagaccttacctgatcttcctccctccatcgctccccgccccccccatccgagcaaccgtcgaccatacg

SEQ ID NO: 16 - DNA, 702 bp, Artificial Sequence
  Synthetic
atgttggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaataa

SEQ ID NO: 17 - DNA, 1000 bp, Nannochloropsis gaditana
  misc_feature: EIF3 promoter
tcataatcaaagatgagccagccacgaagctaccggagaattctgtaagaaaaatgtttaaagttgaaaatgctaacagtgaagtgatatccttttttaatggagtgttgaggtgaagtctagcatcgtaggggaaaacaggattctgtgtcttccattctactccttgataaagcgaagaaatccgacaaaaccaaagagattgttcaagtttaagatttgtaagcgtacaactatgaacttcttctctttgtaggcctgagtggtcgtatgcatacgattcatgaagtgaatcagtatcgctggattttgcttaggagtaaagcacaactaagaaaatatgctgcctggcaggcatcctgagacatgaggcaagcgacgtagcaattgaatcctaatttaagccagggcatctgtatgactctgttagttaattgatgaaccaatgagctttaaaaaaaaatcgttgcgcgtaatgtagttttaattctccgccttgaggtgcggggccatttcggacaaggttctttggacggagatggcagcatgtgtcccttctccaaattggtccgtgtggtagttgagatgctgccttaaaattctgctcggtcatcctgccttcgcattcactcctttcgagctgtcgggttcctcacgaggcctccgggagcggattgcgcagaaaggcgacccggagacacagagaccatacaccgactaaattgcactggacgatacggcatggcgacgacgatggccaagcattgctacgtgattattcgccttgtcattcagggagaaatgatgacatgtgtgggacggtctttacatgggaagagggcatgaaaataacatggcctggcgggatggagcgtcacacctgtgtatgcgttcgatccacaagcaactcaccatttgcgtcggggcctgtctccaatctgctttaggctacttttctctaatttagcctattctatacagacagagacacacagggatc

SEQ ID NO: 18 - DNA, 200 bp, Nannochloropsis gaditana
  misc_feature: bidirectional terminator 5
gggtgggaaggagtcggggagggtcctggcagagcggcgtcctcatgatgtgttggagacctggagagtcgagagcttcctcgtcacctgattgtcatgtgtgtataggttaagggggcccactcaaagccataaagacgaacacaaacactaatctcaacaaagtctactagcatgccgtctgtccatctttatttcct

SEQ ID NO: 19 - protein, 4136 aa, Schizochytrium sp.
  misc_feature: ATCC 208881
MAQPESTTPTMTPEEGQMEGAPQQDNAQVKKHCFADADVATCIAAFGGQGSDWLSELRSLQEKGQTNVRETIELALDKLEDLVKAEPWYEEHGGCDIRAWLESDDNVPNFDLLRYAPVSFPLIFLTQMCNYMRVLEKLGTCHEDALQKGWVKASLGHSQGVVSAAVVAAANTDRELRNLVVSGLEYMSKVGIAAQRTLDYELGRRNAGPETPMLAVQGMDEKVLTKAFKAAVSLSNEKQAMMAKISPTAAAATAAPAAVSDEDRFSIALRNGHDDFVVCGEPKDLRVLRKVIEKQSAEPGKEAQARTPFSKRKPVTQTTFLRMTAVFHSALNKDALAQINTWAPESAFSKAFAQASLRVPVFDTKSGANLQDVPAADVVAHLTTNMLTERADVLVSLRAAETKTDASHLLCFGPGRVAGHLMAHALVGTGIQVVQAADPDTPNDSKRSSAVSIGSVIEAQSPEAVPTAPEWAKKFAPRIAVRAGDGERVLMTKFTSTLGRAPVLMSGMTPTTSFHGVDLVAAASNAGFNAELAAGGLPTPDIFKSKVLELASKLNPGVGISINMLYLNAYQWGFQFPLVVELAKQGVPIESITIGAGVPSEDKAGDIFDGLQSAGINLIAFKPGSKQAIKDVCALAALRPSMNVMLQWTSGRGGGHHSFEDFHEPLLSTYEEIRSHDNIVLVIGSGFGDAQSVLTYLDGSWSQNEIFGRLAKMPVDGVLFGSRCMVALEAATAPEVKQLIVDAEGLEDELSWEKSYEEVAGGVVTVKSELGEPIHKIATRGILLWREFDKRFFDQPRGEKRRNAIMSAKDEIIARLNADFQKPYFGRKSDGSTCDVEEMTYAEVLERMVELMHVKNGGDKAGRLAPTRWIDPTFCSRVLLMMQRAAARFAPKGKKTAVVVPDNKLLKEDPDAAIAKFLDAIPALRESLMADDDVTYFLDLCKVPTRGKPVNFIPVVDEDLGFWLKKDSLWYSEQLDAVPGRDPGRVCILHGPVAARFSTKVDEPIADILGGIHADIVKSIKCDEIKVSVLSPQSLHDASVLRIISETPFLVRGKSFVPNPIGRVVKREGFEHSSFGEDGITVQDPERGLTVATVSQVGSSGTEIEFKVFDKEAGAVLKQTFTVDLTSPRPLFQTEENNLAATKQLYRAAWDCQDEYHAGDTFTDEITVTADNIEAFNLGTSDEYSGSAEAPTDISIMAGWRPLARALFVEELKSNLLKLVHLTNGIRLPNPATRTPVKAGEVIRSEARITGITIQPKVGKKVQVKGRITRAKDSTSEPQMWVEMNSEFLIRGINETPEEYATTFEETPAERHIIEVKDETVAELLMSRAWIKLESGVKIHEGDRVTIDLGTISNRFAGPGRLVDIKASGNVFIETTAKSPRQFPASPSHSAGSVPSDDFINVDSSSGTSKVGVVDFATSDGQEFQVNPVLSFLEKYSEAKNLGHVAENGGYELFDEPAVVKAPADCTTYARGSRDANPIHREEAFAVLADLPDGKPIVHGMWTACMARARLEEIAAKGDLKRIVSYEASFVDMVHCSDELVVTAKQTGVKNGLMLVTVSVNRTSDRALVMTARAELEQPSTAYLFTGQGSASKGMGMDRYAASATVRNVWDRAEDYLRARFGFSILQIVRENPKSFTVHFGGPRGKAIRENLRNLTAQDPSTGQNVPLLPEISATTKSFTFNSPTGLLFATQFSQPALVLVQKAAFEELREAGLVPEKALFAGHSLGEYAALAGYADSLTIEDLVETVFLRGMVMQNAVPRDSEGRSNYAMVAANPLRVGRGFTPEMLSEIVDLITENEEMGKPLLQIVNFNVRFTQYVVAGELLALDALAEALNLAFAKGVRDVAALAEHGAKTAQASLAKRNGRAEPLKRGKATIPLPGIDVPFHSRKLLPGVGAFRKLLAPRFSLQTMEKIIDRLVGNYIPNVTAEVLSLDRAYAEKVQKVTGSQPMAELLEDFDTATDAEKVRTLVIELLAHQFAMPVRWIETQDLMFGSHVERVIEMGPSATLTAMAKQTVKSGAYGDAEEYSPEIMWWKQDRDSVYYEVENVGPSFGDYMDERAALEEELAAAKAAKAEPKAEAASKAEAAPAPKAAAPAPKAAAPAPAAPAPAPAAPAPAPAAGGSVADAPPDAKHVLRALLAAKLKKSMGDVPASTTVQALSAGRSAVQNEVMGELSAEFKGAIPDNAGEMPLAELGGNLSSYKDPGPVTQKLVARTLSAALPGGFGANAAKDYLGQHWGLGAGRTFSVLLHATTMAPEKRLKSEEEGKQWLDDVCKSYGQDAGVSLSPGGGSAGAAAAAPMMMMQQSGPAAAPPPDAPVSALHAMRVMLAAKFEKGFADVADSATVAALSNGKSAVQNEVAGDFGAEFGEVEEAAQTPLSELAGKVQGSYNGPGKVLTRDVNKLLGQCLPGGFGASAARSYLSGDRMLPAGRVESVLIHGLCMAPKGRLGSPEDAKAWLDSLCSAYGSFAGITIPTPGSGGGGAAMGFAGGPQVSSAELSALKSDVQAMVESQLDALRRFLDIDPLHADKLLELEKQVRAETESKLDSIHAEMTVDFCERVQPQFDENRVRVYDSFWNWVVQDAMQMHLHVLGRLEAARQGKEVNASADSNPHFDEMSKWLLGSSSSDAPPIAWFRNFLCNRATPQLLQAVKFFANSMHDAGYVDYAQAISLLAEQVKNWLNNVPVHVANFEPKAPQVRVLDDGVVDYFEIDRDGVNDAARYVAEMSRGLYYKRTGPSRVENPSQTVNVAGDGRLTLLELPPSQSEIEAVAGAGWRLPRSEAELARELTGEPLSLDTSETSSEEGDLPDGPTLDRLRASVNRGVGGDEAEGGEGKISTASLKNGYESIHVSKQVPYVHLKSLSGVDKSVRILNEQLTSEYFSCMDEIATSGVSFAGQVALVTGAGSGSIGAELVKSLLEGGATVLAAIRTARSEAALTKEYARFQSIYKEFGAKDSKLYLVPCNCASQQDMKSLVSYTYDKLGLDVDFVVPFAAAAQQGKDISSIDASSEVSHRMMMTNVVRLLGALKDAKTSRDITTRPAMVLIPCSPNHGEFGQDGLYAESKLGCEALLNKWSSEGWGDYLSLAACVIGWTRSALMQHNNIVAPGIEKLGCRTFAPEETNFNLVGLLHPRMVTLAAEEPLWADLTGCWTVIPNMKDAADSLRSGLMSKSRVARAIAASNQLEEAKSADVSHVLPPPESSGPLASTQLGMTPFPALPSEEARKSLSALEGMVDLRKVVVVVGYGEVGPWGNARTRWEMESYGEFSLEGSIELAWMVGLIKHHDGPLPSGPPRSKYTGWVDAASGEPVADNEIKRRYEEHMLKSCGIRIVEPELFEGYNPEAKRFLHSVVLDRDMPPVELSGLEEGLQYRNELGADCCDVWQKPSDGQWMMRVKKGAEVSIAKALKFNRNIAGQIPTGWDARRFGLPEDIATGVDPVTLYTLVSTVEALMAAGLSDPYELYQYVHVSEVGNTSGGGMGGMRSLKRLFHQRALDEDIPSDTLAESFINTMPAWVNMLLMSSSGPIKTPVGACATAAESLDIGMETILSGKARVVIAGGYDDFGEEGSYEFAQMGATNNTDLDSGRGRTVRESSRPMSSSRAGFVESQGAGMQVLMDAELALEMGAPIFAVLALTSTATDKQGRSIPAPGRGILTSARESSSGAAPSPLLSLERRRAGLKMELEALESLNKQKSDAEGEDAAFFERLIQRRRAAALETWGQGFFKNDPSIAPLRGALAVYGLGVDDLGVASFHGTSTKLNDTNESGVLNKQMEHLGRSKGNVLFVVAQKYLTGHPKGAACAWMLNGLIQCMNDARVPGNRNLDNVDVKLQKNSYLVYPNEPVELPKIEAALLKSFGFGQAGAEVVIVHPDRLLATLSKDVFDKYAETRSLRERRTFRTSQNVMTGVRKYVVVKEAPPYPEELEEAVYLDPLARATYSAASGTWEFRSAAGLTSSGMPRIAAQASGAAQVPAGPTETVAPQGLQVSAKDRLRMTLQEQAEGMSSNFGGSGVGVGVDVENVSTFADFAGSKQDFIARNFTEREIAYCRAAADPAASFAGRWAAKEALVKALSSLAPEQRPLWSGGHAPLVDIEIVPNPSGAPVVQLHGHPQQVSEMLAVNTLSVSISHTADVAVANAVVRK

SEQ ID NO: 20 - protein, 2514 aa, Aquila chrysaetos
  misc_feature: Type I Fatty Acid Synthase (FAS)
MEDVVIAGIAGKLPESENLQEFWENLINGVDMVTEDDRRWKPGIYGLPKRNGKLKDISKFDASFFGVHPKQAHTMDPQLRLLLEVSYEAIMDGGINPATLRGTDTGVWIGASGSEAGEALSQDPVELVGYSMTGCQRAMLANRISYFYDLTGPSLTIDTACSSSLIALENAYKAIRHGWCSAALVGGVNLLLKPNTSVQFMKLGMLSPDGACKAFDASGNGYCRSEAVVVVLLTKRSMAKRIYATIVNAGSNTDGFKEQGVTFPSGEMQQQLVSSLYRECGIEPGEVEYVEAHGTGTKVGDPQEINSIVNVFCQCERAPLLIGSTKSNMGHPESASGLAALAKVILSLEYGLWAPNLHFNTPNPDIPALQDGSLEVVCKPIPVKGGFVSINNFGFGGANAHIILRPNENKHQPLETCNMPRLVQVCGRTQEAVEILIQESRKHGGCSPFASLLSDISVVPVSSMPYRGYTLVGTESDIKEVQHVQASGRPLWYVCSGMGTQWKGMGLSLMKLDLFRQSVLRSDEALKNTGLKVSDLLLQADENTFDDIVQAFVGLAAVQIAQIDMLKAAGLQPDGILGHSVGELACGYADNSLSHEEAILAAYWRGRCVKEAKLPPGGMAAVGLTWEECKQCCPPNVVPACHNSEDTVTVSGPLDSVNEFVAKLKKDGVFAKEVRSAGVAFHSYYMASIAPVLLSALKKVIPDPKPRSARWISTSIPESQWQSDLARTSSAEYHVNNLVSPVLFHEGLKHIPENAVVVEIAPHALLQAILKRALKPTCTILPLMKKEHKNNLEFFLTQTGKIHLTGINVLGNNLFPPVEYPVPVGTPLISPYIKWDHSQDWDVPKAEDFPAGSKGSASASVYNIDASPDSPDHYLVGHCIDGRVLYPATGYLVLAWRTLAQSLGMAMEQMAVMFEEVTIHQATILPKKGSVQLEVRLMPASHCFEVSGNGNLAVSGKISLLENTALKNFHNQLGDFHTQVDMSSKSGLLKEDVYQELHLRGYNYGPTFQGVLECNSEASTGKVLWNGNWVTFLDTLLHVTILAESGRSLRLPTRIRSVYIDPVLHQKQVCQYQDNVEAVDVVVDHCLDSLKAGGVQINGLHASVAPRRQQERIPPTLEKFCFVPYTESNCLSSSVHLRTYLEHCKDLIQNLQAKVAVHGVKLVIPGLKDAVAAAESSPIQKGLQHILAEICRLELNGNLHSELEQIVSQEKMHFQDDPLLNGLLDSSELKTCLDVAVENMTSHRMKIIEALAGSGHLFSRVKSILNAQPLLQVDYIATDCILETLSANETELQDAGVFFSQWDPSSLPSGNLTNADLVVYNCSTSVLGNTTEILSNLAAAVKEGGFVLLHTLLKGETLGEIVSFLTSPDLQQKRSFLSEAQWEGLFSKASLNLVAVKKSFFGSVIFLCRRQVPAKTPIFLPIDETHYKWVESLKEVLADPSEQPVWLTATSCGNSGILGMVNCLRLEAEGHRIRCVFISNLNPSSAVPPMSLSSLEMQKIVQSDLVMNVYRDGKWGSFRHLPLQRAQSQELTEYAYVNVLTRGDLSSLRWIASPLRHFHTTNPNVQLCKVYYASLNFRDIMLATGKLSPDAIPGNWALQQCMLGMEFSGRDLAGRRVMGLLPAKGLATVVDCDKRFLWEVPKNWTLEEAASVPVVYATAYYALVVRGGMKKGESILIHSGSGGVGQAAIAIALSMGCRVFATVGSTEKREYLQARFPQLDANSFASSRNTTFEQHILRVTNGKGVNLVLNSLAEEKLQASLRCLARHGRFLEIGKFDLSNNSQLGMALFLKNVAFHGILLDAIFEAGNQEWEAVSELLKRGIKDGVVKPLRSTVFNKEEVEAAFRFMAQGKHIGKVMIKVQEEEKEYPLRRSEPVKISAISRTSCPSTKSYIITGGLGGFGLELAQWLVERGAQKLILTSRSGIRTGYQAKCVREWKALGIQVLVSTSDIGTLEGTQLLIEEALQLGPVGGIFNLAVVLRDAMIENQTPELFWEVNKPKYSGTLHLDRVTRKKCPDLDYFVVFSSVSCGRGNAGQSNYGFANSTMERICEQRRHDGLPGLAIQWGAIGDVGILLKTMGNREIVVGGTIPQQISSCLEVLDIFLNQPHPVMSSFVLAEKVSVKTEGGSQRDLVEAVAHILGVRDVSSLNAESSLADLGLDSLMGVEVRQTLERDYDIVMTMREIRLLTINKLRELSSKSRTAEELKPSQVMKTGPGKPPKLDLNNLLVNPEGPTITRLNDVQSTERPLFLVHPIEGSIAVFCTLASRLHMPCYGFQCTKAAPLDSIQSLAAYYIDCMKQIQPEGPYRIAGYSFGACVAFEMCSQLQAQQNASHVLNSLFLFDGSHSFVAAYTQSYRAKLTEGNEAALETEALCAFVQQFTGVEYNKLLEVLLPLKDLEARVNAAADLITETYTNINREALSFAAASFYHKLKAADKYIPESKYHGNVTLMRAKSHNEYEEGLGGDYRLSEVCDGKVSVHIIEGDHRTLLEGEGVESIIGIIHSSLAEPRVSVREG

SEQ ID NO: 21 - protein, 292 aa, Aquila chrysaetos
  misc_feature: L-aminoadipate-semialdehyde dehydrogenase- phosphopantetheinyl transferase (PPT)
MSDWLPFLDGGSLPPKDMRMRTLYRTVAEAEGRGSRCRRTALGPMGSVRWAFPCAAWRPCRQEWLLAARLVQPEEKDRVGQFVFARDAKAALAGRLLMRKLIAEKLCIPWNEINLQRTSKGKPFLANNVFSIYSNYNFNVSHQGDYAVLAAEPELQVGIDIMKTNLPGSSSIPNFFHIMKRQFTETEWDVIKSMSNEWMQLDMFYRHWALKESFLKAIGVGIGFNLQRIEFNVSPLQLEIGKVYKETKMLLDGDKEEEWTFEETRLDDHHHVAVALGKQEGFGQKHSDERKT

SEQ ID NO: 22 - protein, 2438 aa, Drosophila melanogaster
  misc_feature: Type I Fatty Acid Synthase (FAS)
MPARFAEEVITAEPAQRAAPQLDLGGGHYVPRQQHLNDEIAITGFSGRLPESSTIEEFKQNLFDGVDMVNDDPRRWERGLYGLPDRIGKLKDSDLENFDQQFFGVHQKQAECMDPLLRMLLELTHEAIIDAGLNPSDLRGSRTGVYIGVSNSETEQHWCSDADRVNGYGLTGCARAMFANRISFTFDFKGPSYSIDTACSSSLYALEQAFSDMREGKVDNALVAGAGLILKPTMSLQFKRLNMLSPDGSCKAFDESGNGYVRSDGCVVLLLQRTSAARRVYASILNVRTNTDGFKEQGITYPIGKMQNRLIRETYEEIGLNPADVVYVEAHGTGTKVGDPQEVNSITDFFCKDRTTPLLIGSVKSNMGHSEPASGVCSVAKILIAMEEGVIPGNLHYNKPNPDLYGLVDGRLKVVDRNLPWNGGIIGLNSFGFGGANAHVILKSNPKPKALTPKDGALKVVLASGRTFEAVEQLLESASTNADDDEYLQLINEIHSKAIPNHFFRGYGVVSSKGTHQREVIESNDDKRPIWYIYSGMGSQWASMAKDLMKIEAFAKTIQRCADVLKPEGVDLIDVLTRSTDKSFENILNSFISIAAMQVALTDLLSSLGIHPDGIVGHSVGELGCAYADGCFTPEQTVLAAYWRGKSILDTQLAKGKMAAVGLSWEDAHSRVPSDCFPVCHNSEDNCTISGPEASIEALVAKLNAEGVFAKAVNSSGYAFHSKYIAEAGPKLRKSLEKIIPNAKNRTARWISTSIPESAWNTPVAKQSSAAYHVNNLLSPVLFHEALQHVPKNAISVEIAPHGLLQAILKRALGPDATNLSLVKRGHENNVEFFLTNVGKLFAAGAQPQVLTLVRPISYPVGRGTPMLNSKVGWDHTQKWLVAKFGKETSSGETIVEVDLSKEDDAFLAGHTIDGRILFPATGYMTLAWQTFAKMQGSEFHKTPVVMENLVFHRATILNKNAVVKFGINFFDGTGAFEICESGSLAVSGKITIPESIDNEELPLEEQTPSAVAKELGTNDVYKELRLRGYDYGGIFRGIVRSDTVASTGKLQWVDNWISFMDTMLQFSILSKNLRELYLPTRIERAVINPAKHFELLSALTKEEQVETGLPVQWYSDINVIKSAGVELRGLKANLAQRRPGTQAPPTLERYQFVPNINTTDLNENSEKARLHALDVAIQVIIENSSGAVKLKGVELANGRNPDVLVANRLLQIIEGEPVLTGDVAVVTSNNNEETITAALGDSGVRVVSKDVLKEPVEQNCHFVFGIDVLSRPDTKTLENSIASIRENGFLILEETLPTYTKTGRALLTKFGFVAVQEQSLGATRVLVLARKAVDLKTRKSVVVVATEQNFNWVDDLKAALATAATEEQYVYVVCQGEELFGAVGLMTCIKNENGGKLARLVFVQDAKAEKFSLTSTLYRQQLEKDLISNVLKNGAWGTFRHLKLETQQATLQVEHAYVNALVKGDLASLKWIEAAQADTAATVDKNLETCTVYYAPINFRDVMLTSGKLAADALPGDLAEQDCVLGLEFAGRDTQGRRVMAMVPAKSLATTCVASKRMMWQIPEKWTMEEASTVPCVYSTVYYALVVRGQMKKGEKILIHAGSGGVGQAAISVALAHGLTVFTTVGSKEKREFLLKRFPKLQERNIGNSRDTSFEQLVLRETKGRGVDLVLNSLSEEKLQASIRCLGLNGRFLEIGKFDLSNNSPLGMSVFLKNTSFHGILLDSVMEGEEEMQNQVVSLVAEGIKTGAVVPLPTSVFNDQQVEQAFRFMASGKHIGKVVIKVRDEEAGKKALQPKPRLINAIPRTYMHPEKSYILVGGLGGFGLELTNWLVTRGARYIVLTSRSGVKTGYQGLMIRRWQERGVKVVIDTSDVTTAAGAKKLLENSNKLALVGGIFNLAAVLRDALIEDQTAKDFKTVADPKVTATKYLDQFSRDICTELDYFICFSSVSCGRGNIGQTNYGLANSAMERICEQRQVSGFPGTAIQWGAIGDTGLVLENLGDNDTVIGGTLPQRMPSCLQTIDLFLQQPHPVVASMVVAEKRKSDQSAGVSLIATIANILGLRDTKNIQDGASLADLGMDSLMSAEIKQTLERNFDIVLSAQEIRQLTFGALKAMDGGADVKPAAAAPAAAAGVPEANITSGGSSRTASPMGDGTQVVFTTSLIPTEAIVQLDTKAPANSKQSPIFFISPIEGFASALEPLAKRLEVPAYGLQYTEAVPSDSLESAAKFFIKQLRTVQPKGPYKLAGYSFGCLLTYVMAGILEETNEVANVIMLDGAPSYVNWYTSSFKQRYTDGTNADNDNQSYGLAYFGIVLANIDYKALVRLLIVIPTWEEKLERFAELMSNEITQPVETIKKSATLFYKKLELADGYQPTLKLKTNVTLVKPTDNSAKLDEDYRLKEVCTKPVEVHTVEGNHRTFLIEDQSLKTIQSILKRLFN

SEQ ID NO: 23 - protein, 293 aa, Drosophila arizonae
  misc_feature: L-aminoadipate-semialdehyde dehydrogenase- phosphopantetheinyl transferase (PPT)
MNKHICSRWAFDLSTWTPTLPQLAHAVAAIQPEERKRLMKFHFIDDFLSSLIGRLLMRKFVSTCSNVPYHQVQFARDVRGKPYWVQDQQSESTPPLSFNVSHQGKLVLLAGVKGDSKDDAEFGIGTDVMKIEYNGGKPLSEFFRLMQSKFSPQEWSYIGRPQHNERAQLKAFMRHWCLKEAYVKELGVGITVDLQKISFAMDTTHPLEEASSPLCGSTLCCNGTPMTQWHFEEHLLTPKYCAAIAFRNCLPLEHAKFEFLPFEELIEQSPKADQPDVVAYCQKALLKPHKRHE

SEQ ID NO: 24 - protein, 2505 aa, Rattus norvegicus
  misc_feature: Type I Fatty Acid Synthase (FAS)
MEEVVIAGMSGKLPESENLQEFWANLIGGVDMVTDDDRRWKAGLYGLPKRSGKLKDLSKFDASFFGVHPKQAHTMDPQLRLLLEVSYEAIVDGGINPASLRGTNTGVWVGVSGSEASEALSRDPETLLGYSMVGCQRAMMANRLSFFFDFKGPSIALDTACSSSLLALQNAYQAIRSGECPAATVGGINLLLKPNTSVQFMKLGMLSPDGTCRSFDDSGNGYCRAEAVVAVLLTKKSLARRVYATILNAGTNTDGCKEQGVTFPSGEAQEQLIRSLYQPGGVAPESLEYIEAHGTGTKVGDPQELNGITRSLCAFRQSPLLIGSTKSNMGHPEPASGLAALTKVLLSLENGVWAPNLHFHNPNPEIPALLDGRLQVVDRPLPVRGGIVGINSFGFGGANVHVILQPNTQQAPAPAPHAALPHLLHASGRTMEAVQGLLEQGRQHSQDLAFVSMLNDIAATPTAAMPFRGYTVLGVEGHVQEVQQVPASQRPLWFICSGMGTQWRGMGLSLMRLDSFRESILRSDEALKPLGVKVSDLLLSTDEHTFDDIVHSFVSLTAIQIALIDLLTSMGLKPDGIIGHSLGEVACGYADGCLSQREAVLAAYWRGQCIKDANLPAGSMAAVGLSWEECKQRCPPGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHVPEHAVVLEIAPHALLQAVLKRGVKPSCTIIPLMKRDHKDNLEFFLTNLGKVHLTGIDINPNALFPPVEFPVPRGTPLISPHIKWDHSQTWDIPVAEDFPNGSSSSSATVYNIDASSESSDHYLVDHCIDGRVLFPGTGYLYLVWKTLARSLSLSLEETPVVFENVTFHQATILPRTGTVPLEVRLLEASHAFEVSDSGNLIVSGKVYQWEDPDSKLFDHPEVPIPAESESVSRLTQGEVYKELRLRGYDYGPHFQGVYEATLEGEQGKLLWKDNWVTFMDTMLQISILGFSKQSLQLPTRVTAIYIDPATHLQKVYMLEGDTQVADVTTSRCLGVTVSGGVYISRLQTTATSRRQQEQLVPTLEKFVFTPHVEPECLSESAILQKELQLCKGLAKALQTKATQQGLKMTVPGLEDLPQHGLPRLLAAACQLQLNGNLQLELGEVLARERLLLPEDPLISGLLNSQALKACIDTALENLSTLKMKVVEVLAGEGHLYSHISALLNTQPMLQLEYTATDRHPQALKDVQTKLQQHDVAQGQWDPSGPAPTNLGALDLVVCNCALATLGDPALALDNMVAALKDGGFLLMHTVLKGHALGETLACLPSEVQPGPSFLSQEEWESLFSRKALHLVGLKKSFYGTALFLCRRLSPQDKPIFLPVEDTSFQWVDSLKSILATSSSQPVWLTAMNCPTSGVVGLVNCLRKEPGGHRIRCILLSNLSSTSHVPKLDPGSSELQKVLESDLVMNVYRDGAWGAFRHFQLEQDKPEEQTAHAFVNVLTRGDLASIRWVSSPLKHMQPPSSSGAQLCTVYYASLNFRDIMLATGKLSPDAIPGKWASRDCMLGMEFSGRDKCGRRVMGLVPAEGLATSVLLSPDFLWDVPSSWTLEEAASVPVVYTTAYYSLVVRGRIQHGETVLIHSGSGGVGQAAISIALSLGCRVFTTVGSAEKRAYLQARFPQLDDTSFANSRDTSFEQHVLLHTGGKGVDLVLNSLAEEKLQASVRCLAQHGRFLEIGKFDLSNNHPLGMAIFLKNVTFHGILLDALFEGANDSWREVAELLKAGIRDGVVKPLKCTVFPKAQVEDAFRYMAQGKHIGKVLVQVREEEPEAMLPGAQPTLISAISKTFCPEHKSYIITGGLGGFGLELARWLVLRGAQRLVLTSRSGIRTGYQAKHVREWRRQGIHVLVSTSNVSSLEGARALIAEATKLGPVGGVFNLAMVLRDAMLENQTPELFQDVNKPKYNGTLNLDRATREACPELDYFVAFSSVSCGRGNAGQSNYGFANSTMERICEQRRHDGLPGLAVQWGAIGDVGIILEAMGTNDTVVGGTLPQRISSCMEVLDLFLNQPHAVLSSFVLVEKKAVAHGDGEAQRDLVKAVAHILGIRDLAGINLDSSLADLGLDSLMGVEVRQILEREHDLVLPIREVRQLTLRKLQEMSSKAGSDTELAAPKSKNDTSLKQAQLNLSILLVNPEGPTLTRLNSVQSSERPLFLVHPIEGSITVFHSLAAKLSVPTYGLQCTQAAPLDSIPNLAAYYIDCIKQVQPEGPYRVAGYSFGACVAFEMCSQLQAQQGPAPAHNNLFLFDGSHTYVLAYTQSYRAKLTPGCEAEAEAEAICFFIKQFVDAEHSKVLEALLPLKSLEDRVAAAVDLITRSHQSLDRRDLSFAAVSFYYKLRAADQYKPKAKYHGNVILLRAKTGGTYGEDLGADYNLSQVCDGKVSVHIIEGDHRTLLEGRGLESIINIIHSSLAEPRVSVREG

SEQ ID NO: 25 - protein, 309 aa, Rattus norvegicus
  misc_feature: L-aminoadipate-semialdehyde dehydrogenase- phosphopantetheinyl transferase (PPT)
MVFPAKRLCVVPYMEGVRWAFSCGTWLPSPAEWLLAVRSIQPEEKERIGQFVFARDAKAALAGRLMIRKLVAEKLNIPWDHIRLQRTSKGKPILAKDTLNPYPNFNFNISHQGDYTVLAAEPELQVGIDIMKTSFPGRGSIPEFFHIMKRKFTNKEWETIRSFNDEWSQLDMFYRHWALKESFIKAIGVGLGFEMQRLEFDVSPLSMDIGQVYKETRLILDGEEEKEWAFEESKIDQHHFVAVALRKPDGSRHQNVSYQDDSKPSQRQFTILNFNDLIASAIPMTPEDPSFWDCFCFTEEILIRNGSKS

(19) United States
(12) Patent Application Publication
(10) Pub. No.: US 2017/0191094 A1
(43) Pub. Date: Jul. 6, 2017

(54) EXPRESSION OF TYPE I FATTY ACID SYNTHASE GENES IN EUKARYOTIC ALGAE

(71) Applicant: Synthetic Genomics, Inc., La Jolla, CA (US)

(72) Inventors: Eric Moellering, San Diego, CA (US); Weidong Xu, San Diego, CA (US); John H. Verruto, San Diego, CA (US); Paul Gordon Roessler, San Diego, CA (US)

(21) Appl. No.: 15/394,414

(22) Filed: Dec. 29, 2016

Related U.S. Application Data
(60) Provisional application No. 62/273,773, filed on Dec. 31, 2015.

(51) Int. Cl. C12P 7/64; C12N 15/82; C12N 9/10

(52) U.S. Cl. CPC C12P 7/6463; C12N 9/1029; C12Y 203/01085; C12N 15/8247

(57) ABSTRACT

The present invention provides recombinant algae expressing exogenous Type I fatty acid synthase (FAS) genes and demonstrating higher rates of fatty acid synthesis with respect to control microorgansims. The recombinant algae are able to produce greater amounts of FAME lipids under nitrogen replete conditions.

### CROSS-REFERENCE TO RELATED APPLICATION

This application claims priority under U.S.C. §119(e) to U.S. Provisional Patent Application No. 62/273,773, filed Dec. 31, 2015, the entire contents of each of which is herein incorporated by reference.

### INCORPORATION OF SEQUENCE LISTING

This application contains references to nucleic acid sequences which have been submitted concurrently herewith as the sequence listing text file “SGI2000_1_Sequence_Listing.txt”, file size 217 kilobytes (kb), created on Dec. 28, 2016. The aforementioned sequence listing is hereby incorporated by reference in its entirety pursuant to 37 C.F.R. §1.52(e)(iii)(5).

### FIELD OF THE INVENTION

The present application relates generally to the field of molecular biology and genetics. Specifically, this application relates to genetically engineered microorganisms such as algae that have increased lipid productivity.

### BACKGROUND OF THE INVENTION

Many microorganisms such as algae, labyrinthulomycetes (“chytrids”), and oleaginous yeast induce lipid biosynthesis in response to nutrient stress, such as nitrogen starvation. Under conditions of nitrogen depletion, such microorganisms redirect compound biosynthesis from protein to storage lipids, typically triacylglyceride lipids (“TAG”). Because nitrogen depletion simultaneously decreases cell growth, optimal lipid biosynthesis is limited to a relatively short window before the cells become too metabolically impaired to maintain high levels of production.

Fatty Acid Synthase (FAS) is an enzyme system occurs in two forms: in prokaryotes and in the chloroplasts of plants and eukaryotic algae, the Type II FAS is a complex of multiple separate enzymes with activities that participate in the two carbon addition to growing acyl chains that originate from the condensation of acetyl-CoA and malonyl-CoA in the cytosol. These acyl chains ultimately form the fatty acid components of membrane lipids and storage lipids. In animals and fungi (as well as heterotrophic heterokonts such as labyrinthulomycetes), a multidomain protein or proteins (Type I FAS) performs these activities. In humans, for example, the multidomain protein complex is formed from two identical 272 kDa polypeptides. Eukaryotic algae produce fatty acids solely in the chloroplast and do not have Type I FAS.

Although the organization of the enzyme functions that form fatty acids in Type I and Type II FAS is quite different, the basic mechanism of elongation and reduction that produces the fatty acyl chains is essentially the same, with the enzyme domains of the Type I systems having a high degree of homology to individual enzymes of Type II FAS.

### SUMMARY OF THE DISCLOSURE

Disclosed herein are recombinant algae that express heterologous Type I Fatty Acid Synthases. A recombinant alga as disclosed herein that includes an exogenous gene encoding a Type I FAS has higher lipid productivity with respect to a control alga (such as a wild type or progenitor alga from which the mutant is derived) cultured under the same conditions. Culture conditions in which a transgenic alga that includes one or more exogenous nucleic acid molecules that encodes a Type I FAS have increased lipid productivity can include nutrient replete conditions and/or nutrient deplete conditions, and can include mixotrophic conditions and/or photoautrophic conditions. The higher lipid productivity can be, for example, higher FAME lipid productivity. In various embodiments a recombinant alga that expresses a heterologous Type I FAS gene produces FAME lipids at a higher rate than a control alga that does not express a heterologous Type I FAS gene, for example, the recombinant alga can produce FAME lipids at a rate at least 5%, at least 10%, at least 15%, at least 20%, at least 25%, at least 30%, at least 35%, at least 40%, at least 45%, at least 50%, at least 60%, at least 70%, at least 80%, at least 90%, or at least 100% higher than a control microorganism that does not express a heterologous Type I FAS gene under mixotrophic or photoautotrophic conditions. Alternatively or in addition, a recombinant alga that expresses a heterologous Type I FAS gene can in some embodiments produce at least 5%, at least 10%, at least 15%, at least 20%, at least 25%, at least 30%, at least 35%, at least 40%, at least 45%, or at least 50% more FAME lipids over a period of from about one hour to at least about twenty-four hours under mixotrophic or photoautotrophic conditions.

A recombinant alga as provided herein that includes an exogenous nucleic acid molecule encoding a Type I FAS can encode a Type I FAS of fungal, labyrinthylomycete, or animal origin, and the gene encoding a Type I FAS or Type I FAS subunit can be codon-optimized or otherwise modified to improve expression of the gene in the algal host. In various embodiments, the recombinant alga includes an exogenous gene that encodes a FAS of an animal, fungal, or labyrinthulomycete species or encodes a FAS derived from a FAS of an animal, fungal, or labyrinthulomycete species that does not include a chloroplast transit peptide or sequence. In some embodiments, the Type I FAS encoded by the exogenous nucleic acid molecule is derived from an animal species, e.g., a species of mammal, fish, reptile, amphibian, bird, marsupial, insect, arachnid, crustacean, etc.. In one example, a Type I FAS produced in an algal cell by expression of an exogenous nucleic acid molecule can be a Danio rerio (zebrafish) Type I FAS or can be derived from a Danio rerio (zebrafish) Type I FAS. In some alternative embodiments, the Type I FAS can be derived from a labyrinthylomycete species. For example, a Type I FAS produced in an algal cell that results in increased production of fatty acids by the algal cell can be a Type I FAS of a Labryinthula, Labryinthuloides, Thraustochytrium, Schizochytrium, Aplanochytrium, Aurantiochytrium, Oblongichytrium, Japonochytrium, Diplophrys, or Ulkenia species, or can be derived from a Type I FAS of a Labryinthula, Labryinthuloides, Thraustochytrium, Schizochytrium, Aplanochytrium, Aurantiochytrium, Oblongichytrium, Japonochytrium, Diplophrys, or Ulkenia species.

In some examples, a recombinant alga as provided herein includes a gene that encodes a Type I FAS polypeptide that has at least 50%, at least 55%, at least 60%, at least 65%, at least 70%, at least 75%, at least 80%, or at least 85%, for example at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity with SEQ ID NO:3, SEQ ID NO:8, SEQ ID NO:19, SEQ ID NO:20, SEQ ID NO:22, or SEQ ID NO:24. In various embodiments the Type I FAS polypeptide does not include a chloroplast transit peptide. The gene can optionally be codon-optimized for the host alga. In some examples the gene can have a coding sequence that has at least 40%, at least 45%, at least 50%, at least 55%, at least 60%, at least 65%, at least 70%, at least 75%, at least 80%, or at least 85%, for example at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO:1, SEQ ID NO:2, or SEQ ID NO:7.

In some examples, for example, where the alga includes an exogenous gene encoding a Type I FAS of an animal species, in addition to an exogenous gene encoding a Type I FAS, the recombinant alga includes an exogenous gene encoding a pantetheine phosphotransferase (PPT). In some examples, the PPT gene encodes a polypeptide that has at least 50%, at least 55%, at least 60%, at least 65%, at least 70%, at least 75%, at least 80%, or at least 85%, for example at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity with SEQ ID NO:6, SEQ ID NO:21, SEQ ID NO:23, or SEQ ID NO:25. The gene can optionally be codon-optimized for the host alga. In some examples the gene can have a coding sequence that has at least 40%, at least 45%, at least 50%, at least 55%, at least 60%, at least 65%, at least 70%, at least 75%, at least 80%, or at least 85%, for example at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO:4 or SEQ ID NO:5.

In various embodiments the recombinant alga that includes an exogenous gene encoding a Type I FAS can further include one or more exogenous genes encoding a gene for making a lipid, including, without limitation, a storage lipid, membrane lipid, or fatty acid derivative. The recombinant alga can produce, for example, a fatty acid, a fatty acid derivative such as a fatty aldehyde, a fatty alcohol, a fatty acid ester, a wax ester, an alkane, or an alkene, or can produce triglycerides. For example, the genetically engineered alga can include one or more nucleic acid molecules encoding one or more polypeptides for producing a fatty acid derivative, such as but not limited to one or more of an exogenous 4-hydroxybenzoyl-CoA thioesterase, acyl-ACP thioesterase, acyl-CoA synthetase, acyl-CoA reductase, carboxylic acid reductase, acyl-ACP reductase, fatty aldehyde reductase, wax synthase, fatty acid decarboxylase, or fatty aldehyde decarbonylase.

In other embodiments the recombinant alga that includes an exogenous gene encoding a Type I FAS can further include one or more exogenous genes encoding a gene for making a storage lipid or membrane lipid. As nonlimiting examples, a polypeptide that participates in the production of a lipid can be an acetyl-CoA carboxylase, a malonyl CoA: ACP transacylase, a beta-ketoacyl-ACP synthase, an acyl-ACP thioesterase, an acyl-CoA thioesterase, a polypeptide having lipolytic activity, a glycerolphosphate acyltransferase (GPAT), a lysophosphatidic acid acyltransferase (LPAAT), a phosphatidic acid phosphatase (PA), or a diacylglycerol 0-acyltransferase (DGAT).

Additionally or alternatively to any of the above additional transgenes, the recombinant alga that includes an exogenous gene encoding a Type I FAS can include mutations that attenuate expression of enzymes involved in beta oxidation of lipids. For example, a recombinant alga as provided herein can have attenuated expression of a native gene encoding an acyl-CoA oxidase.

The algal strain that expresses an exogenous Type I FAS gene can be of any eukaryotic algal species, including, without limitation, a species belonging to any of the genera Achnanthes, Amphiprora, Amphora, Ankistrodesmus, Asteromonas, Boekelovia, Bolidomonas, Borodinella, Botrydium, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Desmodesmus, Dunaliella, Elipsoidon, Emiliania, Eremosphaera, Ernodesmius, Euglena, Eustigmatos, Franceia, Fragilaria, Fragilaropsis, Gloeothamnion, Haematococcus, Hantzschia, Heterosigma, Hymenomonas, Isochrysis, Lepocinclis, Micractinium, Monodus, Monoraphidium, Nannochloris, Nannochloropsis, Navicula, Neochloris, Nephrochloris, Nephroselmis, Nitzschia, Ochromonas, Oedogonium, Oocystis, Ostreococcus, Parachlorella, Parietochloris, Pascheria, Pavlova, Pelagomonas, Phceodactylum, Phagus, Picochlorum, Platymonas, Pleurochrysis, Pleurococcus, Prototheca, Pseudochlorella, Pseudoneochloris, Pseudostaurastrum, Pyramimonas, Pyrobotrys, Scenedesmus, Schizochlamydella, Skeletonema, Spyrogyra, Stichococcus, Tetrachlorella, Tetraselmis, Thalassiosira, Tribonema, Vaucheria, Viridiella, Vischeria, and Volvox.

In some examples, the recombinant alga is a green alga, i.e., an algal member of the Chlorophyte division of the Viridiplantae kingdom, including without limitation, a microalga of any of the classes Chlorophyceae, Chlorodendrophyceae, Pedinophyceae, Pleurastrophyceae, Prasinophyceae, and Trebouxiophyceae. In some examples, a recombinant alga as provided herein can be a species that is a member of any of the Chlorophyceae, Prasinophyceae, Trebouxiophyceae, or Chlorodendrophyceae classes, such as a species of any of the Asteromonas, Ankistrodesmus, Carteria, Chlamydomonas, Chlorococcum, Chlorogonium, Chrysosphaera, Desmodesmus, Dunaliella, Haematococcus, Monoraphidium, Neochloris, Oedogonium, Pelagomonas, Pleurococcus, Pyrobotrys, Scenedesmus, Volvox, Micromonas, Ostreococcus Prasinocladus Scherffelia, Tetraselmis, Botryococcus, Chlorella, Eremosphaera, Franceia, Micractinium, Nannochloris, Oocystis, Parachlorella, Picochlorum, Prototheca, or Pseudochlorella genera. In various examples, a recombinant alga as provided herein can be a species or strain of the Trebouxiophyceae, such as but not limited to Botryococcus, Chlorella, Eremosphaera, Franceia, Micractinium, Nannochloris, Oocystis, Parachlorella, Picochlorum, Prototheca, or Pseudochlorella.

In other examples, the recombinant alga is a heterokont alga, and may belong to the diatoms (bacillariophytes), eustigmatophytes, xanthophytes, phaeophytes, chrysophytes, or raphidophytes. In some examples, the mutant alga belongs to a diatom genus, such as but not limited to a genus selected from the group consisting of Amphiprora, Amphora, Chaetoceros, Cyclotella, Fragilaria, Fragilaropsis, Hantzschia, Navicula, Nitzschia, Phceodactylum, Phceodactylum, Skeletonema, and Thalassiosira. In some examples, the mutant alga is a Eustigmatophyte and belongs to a genus selected from the group consisting of Chloridella, Chlorobptrys, Elhpsoidion, Eustigmatos, Goniochloris, Monodopsis, Monodus, Nannochloropsis, Pseudocharaciopsis, Pseudostaruastrum, Pseudotetraedriella, and Vischeria. In some examples, the mutant alga cell is a Nannochloropsis species.

Also included are methods of producing a lipid that include culturing a recombinant alga such as any disclosed herein that includes an exogenous gene encoding a Type I FAS. The recombinant alga can be cultured in nitrogen deficient conditions or in nitrogen replete culture conditions. The culture conditions may be mixotrophic or photoautotrophic. The method can further comprise isolating lipid from the recombinant alga, the culture medium, or whole culture. The methods can produce fatty acid at a higher rate than is produced by a wild type or control alga.

These and other objects and features of the invention will become more fully apparent when the following detailed description of the invention is read in conjunction with the accompanying drawings.

### BRIEF DESCRIPTION OF THE DRAWINGS

FIG. 1 provides a diagram of a construct for introduction into an algal cell that encodes a Type I FAS derived from Danio rerio (DrFAS) operably linked to a promoter (RPL7 Pro) and terminator (Term 2). The construct also includes a gene encoding a pantetheine phosphotransferase (PPT) from Danio rerio (DrPPT) operably linked to the 4AIII promoter (4AIII Pro) and a terminator (Term 4). The construct further includes a blastocidin resistance gene (B SD) for use as a selectable marker operably linked to a promoter (TCTP Pro) and terminator (EIF3 Term) and a gene encoding a fluorescent protein (GFP) for assessing culture-wide expression of the exogenous FAS and PPT genes operably linked to a promoter (EIF3 Pro) and terminator (Term 5). Promoters and terminators used in the construct are all of algal origin.

FIGS. 2 provide a diagram of a construct for introduction into an algal cell that encodes a Type I FAS (ChytFAS) derived from a labyrinthulomycete species operably linked to a promoter (RPL7 Pro) and terminator (Term 2). The construct further includes a blastocidin resistance gene (BSD) for use as a selectable marker operably linked to a promoter (TCTP Pro) and terminator (EIF3 Term) and a gene encoding a fluorescent protein (GFP) for assessing culture-wide expression of the exogenous FAS gene operably linked to a promoter (EIF3 Pro) and terminator (Term 5). Promoters and terminators used in the construct are all of algal origin.

FIGS. 3A and 3B provide flow cytometry traces (histograms) in which the flow cytometry profile of a wild type WE3730 (non-transformed) algal cell culture (solid lines) is overlaid with the flow cytometry profile of a Nannochloropsis tranformant that includes a Danio rerio Type I FAS gene (dashed lines). The figures also provide Western blots showing levels of FAS protein expression in the profiled transformed lines. A) shows flow cytometry profiles of transformed lines 6200-33, 6200-38, and 6200-43, showing fully penetrant expression with fluorescence peaks that are distinct from the wild type peak (shifted to the right) and protein detected by Western blot. B) shows flow cytometry profiles and Western blots of transformed lines 6201-43, 6201-48, and 6201-38. Line 6201-38 (rightmost flow cytometry trace) shows no difference in its fluorescence profile relative to non-transformed cells and shows no detectable FAS protein in the Western blot (third lane from the right).

FIGS. 4A and 4B provide flow cytometry traces (histograms) of Nannochloropsis transformants in which the flow cytometry profile of a wild type (non-transformed) algal cell culture is overlaid with the flow cytometry profile of a tranformant that includes a Danio rerio Type I FAS gene. The figures also provide Western blots comparing levels of FAS protein expression in the profiled transformed lines. WE3730 is the wild type strain which does not include a Type 1 FAS protein. A) shows flow cytometry profiles of transformed lines 6200-33, 6200-34, and 6200-35, showing fully penetrant expression with fluorescence peaks that are distinct from the wild type peak (shifted to the right) and protein detected by Western blot. B) shows flow cytometry profiles of transformed lines 6200-36, 6200-37, and 6200-38, showing fully penetrant expression with fluorescence peaks that are distinct from the wild type peak (shifted to the right) and protein detected by Western blot.

FIG. 5 provides flow cytometry traces (histograms) of Nannochloropsis transformants in which the flow cytometry profile of a tranformant that includes a labyrinthulomycete Type I FAS gene is overlaid with the flow cytometry profile of a wild type (non-transformed) algal cell culture. The figure also provides a Western blot comparing levels of FAS protein expression in profiled transformed lines 6167-A and 6167-B. WE3730 is the wild type strain which does not include a Type 1 FAS protein.

FIG. 6 provides a graph of FAS activity as assayed from cell extracts of transformants. [Add info on strains that was deleted from figure]

FIGS. 7A and 7B provide graphs of in vivo FAS rate determination using isotope tracer (¹³C) incorporation for ChytFAS transgenic lines A) cultured under phototrophic conditions and B) cultured under mixotrophic conditions. ChytFAS strain 6167-B outperformed the wild type strain under photoautotrophic conditions. Strain 6167-A outperformed wild type in FAME production under mixotrophic conditions.

FIGS. 8A and 8B provides a graph of in vivo FAS rate determination using isotope tracer incorporation for DrFAS over-expression strains A) grown under photoautotrophic conditions where the labeled carbon compound in the culture medium was ¹³C bicarbonate and B) grown under mixotrophic conditions where the labeled carbon compound in the culture medium was ¹³C acetate.

### DETAILED DESCRIPTION

### Definitions

Unless defined otherwise, all technical and scientific terms used herein have the same meaning as commonly understood by one of ordinary skill in the art to which this invention belongs. In case of conflict, the present application including the definitions will control. Unless otherwise required by context, singular terms shall include pluralities and plural terms shall include the singular. All ranges provided within the application are inclusive of the values of the upper and lower ends of the range unless specifically indicated otherwise.

All publications, patents and other references mentioned herein are incorporated by reference in their entireties for all purposes as if each individual publication or patent application were specifically and individually indicated to be incorporated by reference.

The term “and/or” as used in a phrase such as “A and/or B” herein is intended to include “A and B”, “A or B”, “A”, and “B”.

“About” means either within 10% of the stated value, or within 5% of the stated value, or in some cases within 2.5% of the stated value, or, “about” can mean rounded to the nearest significant digit.

The term “gene” is used broadly to refer to any segment of a nucleic acid molecule (typically DNA, but optionally RNA) encoding a polypeptide or expressed RNA. Thus, genes include sequences encoding expressed RNA (which can include polypeptide coding sequences or, for example, functional RNAs, such as ribosomal RNAs, tRNAs, antisense RNAs, microRNAs, short hairpin RNAs, ribozymes, etc.). Genes may further comprise regulatory sequences required for or affecting their expression, as well as sequences associated with the protein or RNA-encoding sequence in its natural state, such as, for example, intron sequences, 5′ or 3′ untranslated sequences, etc. In some examples, “gene” may only refer to a protein-encoding portion of a DNA or RNA molecule, which may or may not include introns. A gene is preferably greater than 50 nucleotides in length, more preferably greater than 100 nucleotide in length, and can be, for example, between 50 nucleotides and 500,000 nucleotides in length, such as between 100 nucleotides and 100,000 nucleotides in length or between about 200 nucleotides and about 50,000 nucleotides in length, or about 200 nucleotides and about 20,000 nucleotides in length. Genes can be obtained from a variety of sources, including cloning from a source of interest or synthesizing from known or predicted sequence information.

The term “nucleic acid” or “nucleic acid molecule” refers to, a segment of DNA or RNA (e.g., mRNA), and also includes nucleic acids having modified backbones (e.g., peptide nucleic acids, locked nucleic acids) or modified or non-naturally-occurring nucleobases. The nucleic acid molecules can be double-stranded or single-stranded; a single stranded nucleic acid molecule that comprises a gene or a portion thereof can be a coding (sense) strand or a non-coding (antisense) strand.

A nucleic acid molecule or polypeptide may be “derived from” an indicated source, which includes the isolation (in whole or in part) of a nucleic acid segment or polypeptide from an indicated source. A nucleic acid molecule may also be derived from an indicated source by, for example, direct cloning, PCR amplification, or artificial synthesis from the indicated polynucleotide source or based on a sequence associated with the indicated polynucleotide source, which may be, for example, a species of organism.

Genes or nucleic acid molecules derived from a particular source or species also include genes or nucleic acid molecules having sequence modifications with respect to the source nucleic acid molecules, that is, the sequence of the gene or nucleic acid molecule is derived from the sequence of a gene or nucleic acid molecule from the referenced source or species but may have modifications. For example, a gene or nucleic acid molecule derived from a source (e.g., a particular referenced gene) can include one or more mutations with respect to the source gene or nucleic acid molecule that are unintended or that are deliberately introduced, and if one or more mutations, including substitutions, deletions, or insertions, are deliberately introduced the sequence alterations can be introduced by random or targeted mutation of cells or nucleic acids, by amplification or other gene synthesis or molecular biology techniques, or by chemical synthesis, or any combination thereof. A gene or nucleic acid molecule that is derived from a referenced gene or nucleic acid molecule that encodes a functional RNA or polypeptide can encode a functional RNA or polypeptide having at least 65%, at least 70%, at least 75%, at least 80%, at least 85%, at least 90%, or at least 95%, sequence identity with the referenced or source functional RNA or polypeptide, or to a functional fragment thereof. For example, a gene or nucleic acid molecule that is derived from a referenced gene or nucleic acid molecule that encodes a functional RNA or polypeptide can encode a functional RNA or polypeptide having at least 85%, at least 90%, at least 95%, at least 96%, at least 97%, at least 98%, or at least 99% sequence identity with the referenced or source functional RNA or polypeptide, or to a functional fragment thereof.

Similarly, a polypeptide or protein derived from a particular source or species includes polypeptides or proteins having sequence modifications with respect to the source prolypeptide, that is, the polpeptide is derived from the sequence of a polypeptide from the referenced source or species but may have modifications. For example, a polypeptide or protein derived from a source (e.g., a particular referenced protein) can include one or more mutations (amino acid differences) with respect to the source polypeptide that are unintended or that are deliberately introduced (for example, by mutation of the encoding nucleic acid molecule). A polypeptide that is derived from a referenced polypeptide can have at least 60%, at least 65%, at least 70%, at least 75%, at least 80%, at least 85%, at least 90%, or at least 95% sequence identity with the referenced or source polypeptide, or to a functional fragment thereof. For example, a polypeptide that is derived from a referenced polypeptide can have at least 80%, or at least 85%, at least 90%, at least 95%, at least 96%, at least 97%, at least 98%, or at least 99% sequence identity with the referenced or source polypeptide, or a functional fragment thereof.

As used herein, an “isolated” nucleic acid or protein is removed from its natural milieu or the context in which the nucleic acid or protein exists in nature. For example, an isolated protein or nucleic acid molecule is removed from the cell or organism with which it is associated in its native or natural environment. An isolated nucleic acid or protein can be, in some instances, partially or substantially purified, but no particular level of purification is required for isolation. Thus, for example, an isolated nucleic acid molecule can be a nucleic acid sequence that has been excised from the chromosome, genome, or episome that it is integrated into in nature.

A “purified” nucleic acid molecule or nucleotide sequence, or protein or polypeptide sequence, is substantially free of cellular material and cellular components. The purified nucleic acid molecule or protein may be substantially free of chemicals beyond buffer or solvent, for example. “Substantially free” is not intended to mean that other components beyond the novel nucleic acid molecules are undetectable.

The terms “naturally-occurring” and “wild type” refer to a form found in nature. For example, a naturally occurring or wild type nucleic acid molecule, nucleotide sequence or protein may be present in and isolated from a natural source, and is not intentionally modified by human manipulation.

As used herein “attenuated” means reduced in amount, degree, intensity, or strength. Attenuated gene expression may refer to a significantly reduced amount and/or rate of transcription of the gene in question, or of translation, folding, or assembly of the encoded protein. As nonlimiting examples, an attenuated gene may be a mutated or disrupted gene (e.g., a gene disrupted by partial or total deletion, truncation, frameshifting, or insertional mutation) that does not encode a complete functional open reading frame or that has decreased expression due to alteration or disruption of gene regulatory sequences. An attenuated gene may also be a gene targeted by a construct that reduces expression of the gene, such as, for example, an antisense RNA, microRNA, RNAi molecule, or ribozyme. Attenuated gene expression can be gene expression that is eliminated, for example, reduced to an amount that is insignificant or undetectable. Attenuated gene expression can also be gene expression that results in an RNA or protein that is not fully functional or nonfunctional, for example, attenuated gene expression can be gene expression that results in a truncated RNA and/or polypeptide.

“Exogenous nucleic acid molecule” or “exogenous gene” refers to a nucleic acid molecule or gene that has been introduced (“transformed”) into a cell. A transformed cell may be referred to as a recombinant cell, into which additional exogenous gene(s) may be introduced. A descendent of a cell transformed with a nucleic acid molecule is also referred to as “transformed” if it has inherited the exogenous nucleic acid molecule. The exogenous gene or nucleic acid molecule may be derived from a different species (and so “heterologous”), or from the same species (and so “homologous”), relative to the cell being transformed. An “endogenous” nucleic acid molecule, gene or protein is a native nucleic acid molecule, gene, or protein as it occurs in, or is naturally produced by, the host.

The term “native” is used herein to refer to nucleic acid sequences or amino acid sequences as they naturally occur in the host. The term “non-native” is used herein to refer to nucleic acid sequences or amino acid sequences that do not occur naturally in the host. Thus, a “non-native” nucleic acid molecule is a nucleic molecule that is not naturally present in the host cell, for example, the non-native nucleic acid molecule is exogenous to the host cell or microorganism into which it is introduced, and may be heterologous with respect to the host cell or microorganism. Additionally, a nucleic acid sequence or amino acid sequence that has been removed from a cell, subjected to laboratory manipulation, and introduced or reintroduced into a host cell such that it differs in sequence or location in the genome with respect to its position in a non-manipulated organism (i.e., is juxtaposed with or operably linked to sequences it is not juxtaposed with or operably linked to in a non-transformed organism) is considered “non-native”. Non-native genes also include genes endogenous to the host microorganism operably linked to one or more heterologous regulatory sequences that have been recombined into the host genome.

A “recombinant” or “engineered” nucleic acid molecule is a nucleic acid molecule that has been altered through human manipulation. As non-limiting examples, a recombinant nucleic acid molecule includes any nucleic acid molecule that: 1) has been partially or fully synthesized or modified in vitro, for example, using chemical or enzymatic techniques (e.g., by use of chemical nucleic acid synthesis, or by use of enzymes for the replication, polymerization, digestion (exonucleolytic or endonucleolytic), ligation, reverse transcription, transcription, base modification (including, e.g., methylation), integration or recombination (including homologous and site-specific recombination) of nucleic acid molecules); 2) includes conjoined nucleotide sequences that are not conjoined in nature; 3) has been engineered using molecular cloning techniques such that it lacks one or more nucleotides with respect to the naturally occurring nucleic acid molecule sequence; and/or 4) has been manipulated using molecular cloning techniques such that it has one or more sequence changes or rearrangements with respect to the naturally occurring nucleic acid sequence. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector.

The term “recombinant protein” as used herein refers to a protein produced by genetic engineering regardless of whether the amino acid varies from that of a wild-type protein.

When applied to organisms, the term recombinant, engineered, or genetically engineered refers to organisms that have been manipulated by introduction of a heterologous or exogenous recombinant nucleic acid sequence into the organism (e.g., a non-native nucleic acid sequence), and includes gene knockouts, targeted mutations, gene replacement, and promoter replacement, deletion, disruption, or insertion, as well as introduction of transgenes or synthetic genes or nucleic acid sequences into the organism. That is, recombinant, engineered, or genetically engineered refers to organisms that have been altered by human intervention. Recombinant or genetically engineered organisms can also be organisms into which constructs for gene “knockdown” have been introduced. Such constructs include, but are not limited to, RNAi, microRNA, shRNA, siRNA, antisense, and ribozyme constructs. Also included are organisms whose genomes have been altered by the activity of meganucleases, zinc finger nucleases, TALENs, or cas/CRISPR systems. An exogenous or recombinant nucleic acid molecule can be integrated into the recombinant/genetically engineered organism's genome or in other instances may not be integrated into the host genome. As used herein, “recombinant microorganism” or “recombinant host cell” includes progeny or derivatives of the recombinant microorganisms of the invention. Because certain modifications may occur in succeeding generations due to either mutation or environmental influences, such progeny or derivatives may not, in fact, be identical to the parent cell, but are still included within the scope of the term as used herein.

The term “promoter” refers to a nucleic acid sequence capable of binding RNA polymerase in a cell and initiating transcription of a downstream (3′ direction) coding sequence. A promoter includes the minimum number of bases or elements necessary to initiate transcription at levels detectable above background. A promoter can include a transcription initiation site as well as protein binding domains (consensus sequences) responsible for the binding of RNA polymerase. Eukaryotic promoters often, but not always, contain “TATA” boxes and “CAT” boxes. Prokaryotic promoters may contain −10 and −35 prokaryotic promoter consensus sequences. A large number of promoters, including constitutive, inducible and repressible promoters, from a variety of different sources are well known in the art. Representative sources include for example, algal, viral, mammalian, insect, plant, yeast, and bacterial cell types, and suitable promoters from these sources are readily available, or can be made synthetically, based on sequences publicly available on line or, for example, from depositories such as the ATCC as well as other commercial or individual sources. Promoters can be unidirectional (initiate transcription in one direction) or bi-directional (initiate transcription in either direction). A promoter may be a constitutive promoter, a repressible promoter, or an inducible promoter. A promoter region can include, in addition to the gene-proximal promoter where RNA polymerase binds to initiate transcription, additional sequences upstream of the gene that can be within 1 kb, 2 kb, 3 kb, 4 kb, 5 kb or more of the transcriptional start site of a gene, where the additional sequences can influence the rate of transcription of the downstream gene and optionally the responsiveness of the promoter to developmental, environmental, or biochemical (e.g., metabolic) conditions.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism species. When referring to a gene regulatory sequence or to an auxiliary nucleic acid sequence used for maintaining or manipulating a gene sequence (e.g. a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e. in the genome of a non-genetically engineered organism) is referred to herein as a “heterologous promoter,” even though the promoter may be derived from the same species (or, in some cases, the same organism) as the gene to which it is linked.

As used herein, the term “protein” or “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms.

Gene and protein Accession numbers, commonly provided in parenthesis after a gene or species name, are unique identifiers for a sequence record publicly available at the National Center for Biotechnology Information (NCBI) website (ncbi.nlm.nih.gov) maintained by the United States National Institutes of Health. The “GenInfo Identifier” (GI) sequence identification number is specific to a nucleotide or amino acid sequence. If a sequence changes in any way, a new GI number is assigned. A Sequence Revision History tool is available to track the various GI numbers, version numbers, and update dates for sequences that appear in a specific GenBank record. Searching and obtaining nucleic acid or gene sequences or protein sequences based on Accession numbers and GI numbers is well known in the arts of, e.g., cell biology, biochemistry, molecular biology, and molecular genetics.

As used herein, the terms “percent identity” or “homology” with respect to nucleic acid or polypeptide sequences are defined as the percentage of nucleotide or amino acid residues in the candidate sequence that are identical with the known polypeptides, after aligning the sequences for maximum percent identity and introducing gaps, if necessary, to achieve the maximum percent homology. N-terminal or C-terminal insertion or deletions shall not be construed as affecting homology, and internal deletions and/or insertions into the polypeptide sequence of less than about 30, less than about 20, or less than about 10 amino acid residues shall not be construed as affecting homology. Homology or identity at the nucleotide or amino acid sequence level can be determined by BLAST (Basic Local Alignment Search Tool) analysis using the algorithm employed by the programs blastp, blastn, blastx, tblastn, and tblastx (Altschul (1997), Nucleic Acids Res. 25, 3389-3402, and Karlin (1990), Proc. Natl. Acad. Sci. USA 87, 2264-2268), which are tailored for sequence similarity searching. The approach used by the BLAST program is to first consider similar segments, with and without gaps, between a query sequence and a database sequence, then to evaluate the statistical significance of all matches that are identified, and finally to summarize only those matches which satisfy a preselected threshold of significance. For a discussion of basic issues in similarity searching of sequence databases, see Altschul (1994), Nature Genetics 6, 119-129. The search parameters for histogram, descriptions, alignments, expect (i.e., the statistical significance threshold for reporting matches against database sequences), cutoff, matrix, and filter (low complexity) can be at the default settings. The default scoring matrix used by blastp, blastx, tblastn, and tblastx is the BLOSUM62 matrix (Henikoff (1992), Proc. Natl. Acad. Sci. USA 89, 10915-10919), recommended for query sequences over 85 in length (nucleotide bases or amino acids).

For blastn, designed for comparing nucleotide sequences, the scoring matrix is set by the ratios of M (i.e., the reward score for a pair of matching residues) to N (i.e., the penalty score for mismatching residues), wherein the default values for M and N can be +5 and −4, respectively. Four blastn parameters can be adjusted as follows: Q=10 (gap creation penalty); R=10 (gap extension penalty); wink=1 (generates word hits at every winkth position along the query); and gapw=16 (sets the window width within which gapped alignments are generated). The equivalent Blastp parameter settings for comparison of amino acid sequences can be: Q=9; R=2; wink=1; and gapw=32. A Bestfit comparison between sequences, available in the GCG package version 10.0, can use DNA parameters GAP=50 (gap creation penalty) and LEN=3 (gap extension penalty), and the equivalent settings in protein comparisons can be GAP=8 and LEN=2.

Thus, when referring to the polypeptide or nucleic acid sequences of the present invention, included are sequence identities of at least 40%, at least 45%, at least 50%, at least 55%, of at least 70%, at least 65%, at least 70%, at least 75%, at least 80%, or at least 85%, for example at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity with the full-length polypeptide or nucleic acid sequence, or to fragments thereof comprising a consecutive sequence of at least 100, at least 125, at least 150 or more amino acid residues of the entire protein; variants of such sequences, e.g., wherein at least one amino acid residue has been inserted N- and/or C-terminal to, and/or within, the disclosed sequence(s) which contain(s) the insertion and substitution. Contemplated variants can additionally or alternately include those containing predetermined mutations by, e.g., homologous recombination or site-directed or PCR mutagenesis, and the corresponding polypeptides or nucleic acids of other species, including, but not limited to, those described herein, the alleles or other naturally occurring variants of the family of polypeptides or nucleic acids which contain an insertion and substitution; and/or derivatives wherein the polypeptide has been covalently modified by substitution, chemical, enzymatic, or other appropriate means with a moiety other than a naturally occurring amino acid which contains the insertion and substitution (for example, a detectable moiety such as an enzyme).

As used herein, the phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz (1979) Principles of Protein Structure, Springer-Verlag). According to such analyses, groups of amino acids can be defined where amino acids within a group exchange preferentially with each other, and therefore resemble each other most in their impact on the overall protein structure (Schulz (1979) Principles of Protein Structure, Springer-Verlag). Examples of amino acid groups defined in this manner can include: a “charged/polar group” including Glu, Asp, Asn, Gln, Lys, Arg, and His; an “aromatic or cyclic group” including Pro, Phe, Tyr, and Trp; and an “aliphatic group” including Gly, Ala, Val, Leu, Ile, Met, Ser, Thr, and Cys. Within each group, subgroups can also be identified. For example, the group of charged/polar amino acids can be sub-divided into sub-groups including: the “positively-charged sub-group” comprising Lys, Arg and His; the “negatively-charged sub-group” comprising Glu and Asp; and the “polar sub-group” comprising Asn and Gln. In another example, the aromatic or cyclic group can be sub-divided into sub-groups including: the “nitrogen ring sub-group” comprising Pro, His, and Trp; and the “phenyl sub-group” comprising Phe and Tyr. In another further example, the aliphatic group can be sub-divided into sub-groups including: the “large aliphatic non-polar sub-group” comprising Val, Leu, and Ile; the “aliphatic slightly-polar sub-group” comprising Met, Ser, Thr, and Cys; and the “small-residue sub-group” comprising Gly and Ala. Examples of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free -OH can be maintained; and Gln for Asn or vice versa, such that a free —NH2 can be maintained. A “conservative variant” is a polypeptide that includes one or more amino acids that have been substituted to replace one or more amino acids of the reference polypeptide (for example, a polypeptide whose sequence is disclosed in a publication or sequence database, or whose sequence has been determined by nucleic acid sequencing) with an amino acid having common properties, e.g., belonging to the same amino acid group or sub-group as delineated above.

As used herein, “expression” includes the expression of a gene at least at the level of RNA production, and an “expression product” includes the resultant product, e.g., a polypeptide or functional RNA (e.g., a ribosomal RNA, a tRNA, an antisense RNA, a micro RNA, an shRNA, a ribozyme, etc.), of an expressed gene. The term “increased expression” includes an alteration in gene expression to facilitate increased mRNA production and/or increased polypeptide expression. “Increased production” [of a gene product] includes an increase in the amount of polypeptide expression, in the level of the enzymatic activity of a polypeptide, or a combination of both, as compared to the native production or enzymatic activity of the polypeptide.

Some aspects of the present invention include the partial, substantial, or complete deletion, silencing, inactivation, or down-regulation of expression of particular polynucleotide sequences. The genes may be partially, substantially, or completely deleted, silenced, inactivated, or their expression may be down-regulated in order to affect the activity performed by the polypeptide they encode, such as the activity of an enzyme. Genes can be partially, substantially, or completely deleted, silenced, inactivated, or down-regulated by insertion of nucleic acid sequences that disrupt the function and/or expression of the gene (e.g., viral insertion, transposon mutagenesis, meganuclease engineering, homologous recombination, or other methods known in the art). The terms “eliminate,” “elimination,” and “knockout” can be used interchangeably with the terms “deletion,” “partial deletion,” “substantial deletion,” or “complete deletion.” In certain embodiments, a microorganism of interest may be engineered by site directed homologous recombination or targeted integration or mutation using a cas/CRISPR system to knockout a particular gene of interest. In still other embodiments, targeted insertion into or mutation of a gene regulatory region using a cas/CRISPR system, RNAi, or antisense DNA (asDNA) constructs may be used to partially, substantially, or completely silence, inactivate, or down-regulate a particular gene of interest.

These insertions, deletions, or other modifications of certain nucleic acid molecules or particular polynucleotide sequences may be understood to encompass “genetic modification(s)” or “transformation(s)” such that the resulting strains of the microorganisms or host cells may be understood to be “genetically modified”, “genetically engineered” or “transformed.”

As used herein, “up-regulated” or “up-regulation” includes an increase in expression of a gene or nucleic acid molecule of interest or the activity of an enzyme, e.g., an increase in gene expression or enzymatic activity as compared to the expression or activity in an otherwise identical gene or enzyme that has not been up-regulated.

As used herein, “down-regulated” or “down-regulation” includes a decrease in expression of a gene or nucleic acid molecule of interest or the activity of an enzyme, e.g., a decrease in gene expression or enzymatic activity as compared to the expression or activity in an otherwise identical gene or enzyme that has not been down-regulated.

As used herein, “mutant” refers to an organism that has a mutation in a gene that is the result of classical mutagenesis, for example, using gamma irradiation, UV, or chemical mutagens. “Mutant” as used herein also refers to a recombinant cell that has altered structure or expression of a gene as a result of genetic engineering that many include, as non-limiting examples, overexpression, including expression of a gene under different temporal, biological, or environmental regulation and/or to a different degree than occurs naturally and/or expression of a gene that is not naturally expressed in the recombinant cell; homologous recombination, including knock-outs and knock-ins (for example, gene replacement with genes encoding polypeptides having greater or lesser activity than the wild type polypeptide, and/or dominant negative polypeptides); gene attenuation via RNAi, antisense RNA, or ribozymes, or the like; and genome engineering using meganucleases, TALENs, and/or CRISPR technologies, and the like. A mutant is therefore not a naturally-occurring organism. A mutant organism of interest will typically have a phenotype different than that of the corresponding wild type or progenitor strain that lacks the mutation, where the phenotype can be assessed by growth assays, product analysis, photosynthetic properties, biochemical assays, etc. When referring to a gene “mutant” means the gene has at least one base (nucleotide) change, deletion, or insertion with respect to a native or wild type gene. The mutation (change, deletion, and/or insertion of one or more nucleotides) can be in the coding region of the gene or can be in an intron, 3′ UTR, 5′ UTR, or promoter region, e.g., within 2 kb of the transcriptional start site or within 3 kb or the translational start site. For example, a mutant having attenuated expression of a gene as disclosed herein can have a mutation, which can be one or more nucleobase changes and/or one or more nucleobase deletions and/or one or more nucleobase insertions, into the region of a gene 5′ of the transcriptional start site, such as, in non-limiting examples, within 2 kb, within 1.5 kb, within 1kb, or within 0.5 kb of the known or putative transcriptional start site, or within 3 kb, within 2.5 kb, within 2kb, within 1.5 kb, within 1kb, or within 0.5 kb of the translational start site. As nonlimiting examples, a mutant gene can be a gene that has a mutation, insertion, or deletion within the promoter region that can either increase or decrease expression of the gene; can be a gene that has a deletion that results in production of a nonfunctional protein, truncated protein, dominant negative protein, or no protein; can be a gene that has one or more point mutations leading to a change in the amino acid of the encoded protein or results in aberrant splicing of the gene transcript, etc.

Conserved domains of polypeptides include those identified in the “cd” (conserved domain) database, the COG database, the SMART database, the PRK database, the TIGRFAM database, or others known the art. The National Center for Biotechnology Information website (ncbi.nlm.nih.gov/Structure/cdd/wrpsb.cgi) sponsored by the U.S. National Institutes of Health includes a conserved domain database (CDD) which it describes as “a protein annotation resource that consists of a collection of well-annotated multiple sequence alignment models for ancient domains and full-length proteins. These are available as position-specific score matrices (PSSMs) for fast identification of conserved domains in protein sequences via RPS-BLAST. CDD content includes NCBI-curated domains, which use 3D-structure information to explicitly define domain boundaries and provide insights into sequence/structure/function relationships, as well as domain models imported from a number of external source databases (Pfam, SMART, COG, PRK, TIGRFAM).”

The term “Pfam” refers to a large collection of protein domains and protein families maintained by the Pfam Consortium and available at several sponsored world wide web sites, including: pfam.sanger.ac.uk/(Welcome Trust, Sanger Institute); pfam.sbc.su.se (Stockholm Bioinformatics Center); pfam.janelia.org/(Janelia Farm, Howard Hughes Medical Institute); pfam.jouy.inra.fr/(Institut national de la Recherche Agronomique); and pfam.ccbb.re.kr. The latest release of Pfam is Pfam 30.0 (May 2016). Pfam domains and families are identified using multiple sequence alignments and hidden Markov models (HMMs). Pfam-A family or domain assignments, are high quality assignments generated by a curated seed alignment using representative members of a protein family and profile hidden Markov models based on the seed alignment. (Unless otherwise specified, matches of a queried protein to a Pfam domain or family are Pfam-A matches.) All identified sequences belonging to the family are then used to automatically generate a full alignment for the family (Sonnhammer (1998) Nucleic Acids Research 26, 320-322; Bateman (2000) Nucleic Acids Research 26, 263-266; Bateman (2004) Nucleic Acids Research 32, Database Issue, D138-D141; Finn (2006) Nucleic Acids Research Database Issue 34, D247-251; Finn (2010) Nucleic Acids Research Database Issue 38, D211-222). By accessing the Pfam database, for example, using any of the above-reference websites, protein sequences can be queried against the HMMs using HMMER homology search software (e.g., HMMER2, HMMER3, or a higher version, hmmer.janelia.org/). Significant matches that identify a queried protein as being in a pfam family (or as having a particular Pfam domain) are those in which the bit score is greater than or equal to the gathering threshold for the Pfam domain. Expectation values (e values) can also be used as a criterion for inclusion of a queried protein in a Pfam or for determining whether a queried protein has a particular Pfam domain, where low e values (much less than 1.0, for example less than 0.1, or less than or equal to 0.01) represent low probabilities that a match is due to chance.

A “cDNA” is a DNA molecule that comprises at least a portion the nucleotide sequence of an mRNA molecule, with the exception that the DNA molecule substitutes the nucleobase thymine, or T, in place of uridine, or U, occurring in the mRNA sequence. A cDNA can be double stranded or single stranded and can be, for example, the complement of the mRNA sequence. In preferred examples, a cDNA does not include one or more intron sequences that occur in the naturally-occurring gene that the cDNA corresponds to (i.e., the gene as it occurs in the genome of an organism). For example, a cDNA can have sequences from upstream of an intron of a naturally-occurring gene juxtaposed to sequences downstream of the intron of the naturally-occurring gene, where the upstream and downstream sequences are not juxtaposed in a DNA molecule in nature (i.e., the sequences are not juxtaposed in the naturally occurring gene). A cDNA can be produced by reverse transcription of mRNA molecules, or can be synthesized, for example, by chemical synthesis and/or by using one or more restriction enzymes, one or more ligases, one or more polymerases (including, but not limited to, high temperature tolerant polymerases that can be used in polymerase chain reactions (PCRs)), one or more recombinases, etc., based on knowledge of the cDNA sequence, where the knowledge of the cDNA sequence can optionally be based on the identification of coding regions from genome sequences or compiled from the sequences multiple partial cDNAs.

Reference to properties that are “substantially the same” or “substantially identical” without further explanation of the intended meaning, is intended to mean the properties are within 10%, and preferably within 5%, and may be within 2.5%, of the reference value. Where the intended meaning of “substantially” in a particular context is not set forth, the term is used to include minor and irrelevant deviations that are not material to the characteristics considered important in the context of the invention.

A “control cell” or “control microorganism” is either a wild type cell or microorganism from which the mutant microorganism (genetically engineered or mutagenized microorganism) is directly or indirectly derived, or is a cell or microorganism that is substantially identical to the mutant cell or microorganism referred to, with the exception that the control cell or microorganism does not have the mutation resulting in increased lipid production, for example, the control cell or microorganism has not been genetically engineered or mutagenized to increase lipid production. For example, where the recombinant alga comprises an exogenous gene encoding a Type I FAS, a control alga can be substantially identical to the recombinant alga with the exception that the control alga does not comprise an exogenous gene encoding a Type I FAS.

“The same conditions” or “the same culture conditions”, as used herein, means substantially the same conditions, that is, any differences between the referenced conditions that may be present are minor and not relevant to the function or properties of the microorganism that are material to the invention, including lipid production or biomass production.

As used herein “lipid” or “lipids” refers to fats, waxes, fatty acids, fatty acid derivatives such as fatty alcohols, wax esters, alkanes, and alkenes, sterols, monoglycerides, diglycerides, triglycerides, phospholipids, sphingolipids, saccharolipids, and glycerolipids. “FAME lipids” or “FAME” refers to lipids having acyl moieties that can be derivatized to fatty acid methyl esters, such as, for example, monoacylglycerides, diacylglycerides, triacylglycerides, wax esters, and membrane lipids such as phospholipids, galactolipids, etc. Lipid productivity can be assessed as FAME productivity in milligrams per liter (mg/L) and for algae, may be reported as grams per meter² per day (g/m²/day). In the semi-continuous assays provided herein, mg/L values are converted to g/m2/day by taking into account the area of incident irradiance (the SCPA flask rack aperture of 1½″×33/8″, or 0.003145m²) and the volume of the culture (550 ml ). To obtain productivity values in g/m²/day, mg/L values are multiplied by the daily dilution rate (30%) and a conversion factor of 0.175. Where lipid or subcategories thereof (for example, TAG or FAME) are referred to as a percentage, the percentage is a weight percent unless indicated otherwise.

As used herein, the term “fatty acid product” includes free fatty acids, mono-di- or triglycerdes, fatty aldehydes, fatty alcohols, fatty acid esters (including, but not limited to, wax esters); and hydrocarbons, including, but not limited to, alkanes and alkenes).

“Biomass” refers to cellular mass, whether of living or dead cells, and can be assessed, for example, as aspirated pellet weight, but is more preferably dry weight (e.g., lyophilate of a culture sample or pelleted cells), ash-free dry weight (AFDW), or total organic carbon (TOC), using methods known in the art. Biomass increases during the growth of a culture under growth permissive conditions and may be referred to as “biomass accumulation” in batch cultures, for example. In continuous or semi-continuous cultures that undergo steady or regular dilution, biomass that is produced that would otherwise accumulate in the culture is removed during culture dilution. Thus, daily biomass productivity (increases in biomass) by these cultures can also be referred to as “biomass accumulation”. Biomass productivity can be assessed as TOC productivity in milligrams per liter (mg/L) and for algae, may be reported as grams per meter² per day (g/m²/day). In the semi-continuous assays provided herein, mg/L values are converted to g/m2/day by taking into account the area of incident irradiance (the SCPA flask rack aperture of 1½″×33/8″, or 0.003145m²) and the volume of the culture (550 ml ). To obtain productivity values in g/m²/day, mg/L values are multiplied by the daily dilution rate (30%) and a conversion factor of 0.175. Where biomass is expressed as a percentage, the percentage is a weight percent unless indicated otherwise.

In the context of the invention, a “nitrogen source” is a source of nitrogen that can be taken up and metabolized by the subject microorganism and incorporated into biomolecules for growth and propagation. For example, compounds including nitrogen that cannot be taken up and/or metabolized by the microorganism for growth (e.g., nitrogen-containing biological buffers such as Hepes, Tris, etc.) are not considered nitrogen sources in the context of the invention.

“Reduced nitrogen”, as used herein, is nitrogen in the chemical form of ammonium, ammonia, urea, or an amino acid that can be taken up and metabolized by the microorganism being cultured to provide a source of nitrogen for incorporation into biomolecules, thereby supporting growth. For example, in addition to ammonium/ammonia and urea, reduced nitrogen can include various amino acids where the amino acid(s) can serve as a nitrogen source to the subject microorganism. Examples of amino acids can include, without limitation, glutamate, glutamine, histidine, lysine, arginine, asparagine, alanine, and glycine. “Non-reduced nitrogen” in the context of a nitrogen source that can be present in a culture medium for microorganisms refers to nitrate or nitrite that must be reduced prior to assimilation into organic compounds by the microorganism.

“The sole source of nitrogen [in the culture medium]” is used interchangeably with “substantially the sole source of nitrogen” and indicates that no other nitrogen source is intentionally added to the culture medium, or that no other nitrogen source is present in an amount sufficient to significantly increase the growth of the microorganisms or cells cultured in the referenced medium. Throughout this application, for brevity, the terms “nitrate-only” is used to characterize culture media in which nitrate is the only source of nitrogen that is available to the microorganisms for supporting growth.

Similarly, “the sole source of carbon [in the culture medium]” is used interchangeably with “substantially the sole source of carbon” and indicates that no other carbon source is present in an amount sufficient to increase the productivity, growth, or propagation of the microorganisms or cells cultured in the referenced medium or become incorporated into biomolecules such as lipids produced by the microorganisms or cells.

“Nitrogen replete” conditions refer to media conditions in which no further growth or propagation benefit is conferred by adding additional nitrogen (in a form that can be used by the microorganism) to the medium. Similarly, “nutrient replete” conditions refer to media conditions in which no nutrient is limiting to growth or propagation, that is, when a medium is nutrient replete, adding additional nutrient(s) to the medium does not result in an improved growth or propagation rate. In the context of “nutrient replete”, “nutrients” includes, as nonlimiting examples, phosphate, sulfur, iron, and optionally silica, but excludes carbon sources such as sugars or organic acids that may be used by the organism as an energy source.

Disclosed herein are methods for manipulating, assaying, culturing, and analyzing microorganisms. The invention set forth herein also makes use of standard methods, techniques, and reagents for cell culture, transformation of microorganisms, genetic engineering, and biochemical analysis that are known in the art. Although methods and materials similar or equivalent to those described herein can be used in practice or testing of the present invention, suitable methods and materials are described below. The materials, methods, and examples are illustrative only and are not intended to be limiting. Other features and advantages of the invention will be apparent from the detailed description and from the claims.

### Recombinant Algae Having Exogenous Genes Encoding Type I FAS

The invention provides recombinant algae that include at least one exogenous nucleic acid sequence encoding a Type I FAS. A recombinant alga expressing an exogenous Type I FAS gene can be a recombinant alga, for example, a recombinant eukaryotic alga, such as a eukaryotic microalga, which is used herein to denote a eukaryotic unicellular alga. The exogenous nucleic acid sequence encoding a Type I FAS is introduced by human intervention, and, as algae, such as eukaryotic algae, do naturally have Type I FAS genes, the nucleic acid sequence is also heterologous with respect to the algal host, i.e., not derived from a gene native to the algal host.

The exogenous (i.e., introduced) and heterologous (non-algal) gene encoding the Type I FAS can encode any Type I FAS, such as a Type I FAS of animal, fungal, or labyrinthulomycete origin. For example the gene can encode a Type I FAS having at least 65%, at least 70%, at least 75%, at least 80%, at least 85%, at least 90%, or at least 95% sequence identity with a naturally-occurring FAS polypeptide, for example, at least 85%, at least 90%, at least 95%, at least 96%, at least 97%, at least 98%, or at least 99% sequence identity with a naturally-occurring FAS polypeptide. The Type I FAS gene in various preferred embodiments does not include an amino acid sequence targeting the Type I FAS to the chloroplast, i.e., does not include a chloroplast transit peptide. For example,because Type I FAS genes and polypeptides do not naturally occur in photosynthetic organisms, a Type I FAS will not naturally include a chloroplast transit peptide for localization of the FAS to the chloroplast. Further, in various embodiments the Type I FAS is not engineered to include a chloroplast transit peptide. The heterologous Type I FAS expressed in the algal cell can in various embodiments be localized in the cytoplasm of the cell.

The transgenic alga that includes an exogenous Type I FAS gene can further include an exogenous gene encoding a L-aminoadipate-semialdehyde dehydrogenase-phosphopantetheinyl transferase, referred to as “pantetheine phosphotransferase” or “PPT”. For example, in embodiments where the alga includes a transgene encoding a Type I FAS of animal origin (or Type I FAS derived from a Type I FAS of an animal species), the alga can also preferably include an exogenous PPT gene. The PPT gene can encode, for example, a PPT that is at least 75%, at least 80%, at least 85%, at least 90%, at least 95% identical to a naturally-occurring PPT.

The recombinant alga that includes an exogenous (and heterologous) gene encoding a Type I FAS can demonstrate higher fatty acid productivity than is demonstrated by a control alga that does not include an exogenous nucleic acid sequence encoding a Type I FAS. For example, a recombinant alga that includes a gene encoding a heterologous Type I Fatty Acid Synthase can produce fatty acids and/or lipids at a higher rate than a control microorganisms (such as wild type microorganisms from which the mutants are derived) cultured under the same conditions, including nutrient replete conditions, which may be photoautrophic conditions or mixotrophic conditions. For example, the recombinant alga can produce FAME lipids at a rate at least 5%, at least 10%, at least 15%, at least 20%, at least 25%, at least 30%, at least 35%, at least 40%, at least 45%, at least 50%, at least 60%, at least 70%, at least 80%, at least 90%, or at least 100% higher than a control microorganism that does not express a heterologous Type I FAS gene under mixotrophic or photoautotrophic conditions. The rate of fatty acid or lipid production can be, for example, between 5% and 200% higher than the rate of lipid or fatty acid production by a control alga, or between 10% and 200% higher than the rate of lipid or fatty acid production by a control alga, between 10% and 150% higher than the rate of lipid or fatty acid production by a control alga, or, for example, between 10% and 100% higher than the rate of lipid or fatty acid production by a control alga, or between 10% and 70% higher than the rate of lipid or fatty acid production by a control alga. The lipid can be, for example, fatty acid methyl ester-derivitizable lipid, i.e., FAME lipids or “FAME”. A recombinant alga that expresses a heterologous Type I FAS gene can in some examples have higher fatty acid or FAME productivity than a control alga that does not express a heterologous Type I FAS gene, for example, can have at least 5%, at least 10%, at least 15%, at least 20%, %, at least 25%, at least 30%, at least 35%, at least 40%, at least 45%, at least 50%, at least 60%, at least 70%, at least 80%, at least 90%, or at least 100% higher fatty acid or FAME productivity over a period of from about one hour to about twenty-four hours under mixotrophic or photoautotrophic conditions, e.g., between 5% and 200% higher FAME productivity than a control alga, or between 10% and 200% higher FAME productivity than a control alga, between 10% and 150% higher FAME productivity than a control alga, or, for example, between 10% and 100% higher FAME productivity than a control alga, or between 10% and 70% higher FAME productivity than a control alga.

Productivity can be volumetric productivity, for example, the productivity of a culture can be expressed as weight per milliliter or liter of culture, and can be a daily productivity (e.g., mg/liter/day or g/liter/day), for example, an average daily productivity over multiple days of the culture (for example, at least three, four, five, six, seven, eight, nine, ten, eleven, twelve, thirteen, fourteen fifteen, or more days), or can be a total amount produced per unit volume for a defined period of time in culture. Productivity may be measured multiple times during the culture period, for example, at least twice or at least three times, and may be assessed every day, every other day, every third day, etc.

Biomass productivity can be assessed, for example, by measuring total organic carbon (TOC) or by other methods, such as measuring dry weight, ash-free dry weight (AFDW). Methods for measuring TOC are known in the art (e.g., U.S. Pat. No. 8,835,149) and are provided herein. Methods of measuring AFDW are also well-known and can be found, for example, in U.S. Pat. No. 8,940,508, incorporated herein by reference in its entirety.

Methods of measuring the amount of lipid produced by microorganisms are also well-known in the art and provided in the examples herein. For example, total extractable lipid can be determined according to Folch et al. (1957) 1 Biol. Chem. 226: 497-509; Bligh & Dyer (1959) Can. I Biochem. Physiol. 37: 911-917; or Matyash et al. (2008) 1 Lipid Res. 49:1137-1146, for example, and the percentage of biomass present as lipid can also be assessed using Fourier transform infrared spectroscopy (FT-IR) (Pistorius et al. (2008) Biotechnol & Bioengin. 103:123-129). Additional references for gravimetric analysis of FAME and TAGs are provided in U.S. Pat. No. 8,207,363 and WO 2011127118 for example, each incorporated herein by reference in its entirety.

FAS activity and fatty acid productivity can also be assessed using methods disclosed in Example 3 herein, for example, using cell extracts and in vivo labeling.

In various embodiments the recombinant alga that includes an exogenous gene encoding a Type I FAS can further include one or more exogenous genes encoding a gene for making a lipid, including, without limitation, a storage lipid, membrane lipid, or fatty acid derivative. The recombinant alga can produce, for example, a fatty acid, a fatty acid derivative such as a fatty aldehyde, a fatty alcohol, a fatty acid ester, a wax ester, an alkane, or an alkene, or can produce triglycerides. For example, the genetically engineered alga can include one or more nucleic acid molecules encoding one or more polypeptides for producing a fatty acid derivative, such as but not limited to one or more of an exogenous 4-hydroxybenzoyl-CoA thioesterase, acyl-ACP thioesterase, acyl-CoA synthetase, acyl-CoA reductase, carboxylic acid reductase, acyl-ACP reductase, fatty aldehyde reductase, wax synthase, fatty acid decarboxylase, or fatty aldehyde decarbonylase.

In other embodiments the recombinant alga that includes an exogenous gene encoding a Type I FAS can further include one or more exogenous genes encoding a gene for making a storage lipid or membrane lipid. As nonlimiting examples, a polypeptide that participates in the production of a lipid can be an acetyl-CoA carboxylase, a malonyl CoA: ACP transacylase, a beta-ketoacyl-ACP synthase, an acyl-ACP thioesterase, an acyl-CoA thioesterase, a polypeptide having lipolytic activity, a glycerolphosphate acyltransferase (GPAT), a lysophosphatidic acid acyltransferase (LPAAT), a phosphatidic acid phosphatase (PA), or a diacylglycerol O-acyltransferase (DGAT).

The alga can further have one or more genetic modifications that result in attenuated expression of a gene that encodes an enzyme that participates in fatty acid oxidation such as an acyl-CoA oxidase.

Recombinant algae can be, in various examples, of any eukaryotic microalgal strain such as, for example, any species of any of the genera Achnanthes, Amphiprora, Amphora, Ankistrodesmus, Asteromonas, Boekelovia, Bolidomonas, Borodinella, Botrydium, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Desmodesmus, Dunaliella, Elipsoidon, Emiliania, Eremosphaera, Ernodesmius, Euglena, Eustigmatos, Franceia, Fragilaria, Fragilaropsis, Gloeothamnion, Haematococcus, Hantzschia, Heterosigma, Hymenomonas, Isochrysis, Lepocinclis, Micractinium, Monodus, Monoraphidium, Nannochloris, Nannochloropsis, Navicula, Neochloris, Nephrochloris, Nephroselmis, Nitzschia, Ochromonas, Oedogonium, Oocystis, Ostreococcus, Parachlorella, Parietochloris, Pascheria, Pavlova, Pelagomonas, Phceodactylum, Phagus, Picochlorum, Platymonas, Pleurochrysis, Pleurococcus, Prototheca, Pseudochlorella, Pseudoneochloris, Pseudostaurastrum, Pyramimonas, Pyrobotrys, Scenedesmus, Schizochlamydella, Skeletonema, Spyrogyra, Stichococcus, Tetrachlorella, Tetraselmis, Thalassiosira, Tribonema, Vaucheria, Viridiella, Vischeria, and Volvox. Non-limiting examples of particularly suitable species include, for instance, diatoms such as, for example, a species of any of the genera Amphora, Chaetoceros, Cyclotella, Fragilaria, Fragilaropsis, Hantzschia, Monodus, Navicula, Nitzschia, Phceodactylum, or Thalassiosira, or Eustigmatophytes, e.g., Eustigmatos, Monodus, Nannochloropsis, Pseudostaurastrum, or Vischeria. In some exemplary embodeiments the algae are of the genus Nannochloropsis.

One skilled in the art will appreciate that a number of transformation methods can be used for genetic transformation of microorganisms and, therefore, can be deployed for the methods of the present invention. “Stable transformation” is intended to mean that the nucleic acid construct introduced into an organism integrates into the genome of the organism or is part of a stable episomal construct and is capable of being inherited by the progeny thereof. “Transient transformation” is intended to mean that a polynucleotide is introduced into the organism and does not integrate into the genome or otherwise become established and stably inherited by successive generations.

Genetic transformation can result in stable insertion and/or expression of transgenes, constructs from either the nucleus or the plastid, and in some cases can result in transient expression of transgenes. The transformation methods can also be used for the introduction of guide RNAs or editing DNAs. Genetic transformation of microalgae has been reported successful for more than 30 different strains of microalgae, which belong to at least ˜22 species of green, red, and brown algae, diatoms, euglenids, and dianoflagellates (see, e.g., Radakovits et al., Eukaryotic Cell, 2010; and Gong et al., J. Ind. Microbiol. Biotechnol., 2011). Non-limiting examples of such useful transformation methods include agitation of cells in the presence of glass beads or silicon carbide whiskers as reported by, for example, Dunahay, Biotechniques, 15(3):452-460, 1993; Kindle, Proc. Natl. Acad. Sci. U.S.A., 1990; Michael and Miller, Plant J., 13, 427-435, 1998. Electroporation techniques have been successfully used for genetic transformation of several microalgal species including Nannochloropsis sp. (see, e.g., Chen et al., J. Phycol., 44:768-76, 2008), Chlorella sp. (see, e.g., Chen et al., Curr. Genet., 39:365-370, 2001; Chow and Tung, Plant Cell Rep. Vol.18, No. 9, 778-780, 1999), Chlamydomonas (Shimogawara et al., Genetics, 148: 1821-1828, 1998), Dunaliella (Sun et al., Mol. Biotechnol., 30(3): 185-192, 2005). Micro-projectile bombardment, also referred to as microparticle bombardment, gene gun transformation, or biolistic bombardment, has been used successfully for several algal species including, for example, diatoms species such as Phaeodactylum (Apt et al., Mol. Gen. Genet., 252:572-579, 1996), Cyclotella and Navicula (Dunahay et al., J. Phycol., 31:1004-1012, 1995), Cylindrotheca (Fischer et al., J. Phycol., 35:113-120, 1999), and Chaetoceros sp. (Miyagawa-Yamaguchi et al., Phycol. Res. 59: 113-119, 2011), as well as green algal species such as Chlorella (El-Sheekh, Biologia Plantarum, Vol.42, No.2: 209-216, 1999), and Volvox species (Jakobiak et al., Protist, 155:381-93, 2004). Additionally, Agrobacterium-mediated gene transfer techniques can also be useful for genetic transformation of microalgae, as has been reported by, for example, Kumar, Plant Sci., 166(3):731-738, 2004, and Cheney et al., J. Phycol., Vol. 37, Suppl. 11, 2001.

A transformation vector or construct as described herein will typically comprise a marker gene that confers a selectable or scorable phenotype on target host cells, e.g., algal cells or may be co-transformed with a construct that includes a marker. A number of selectable markers have been successfully developed for efficient isolation of genetic transformants of algae. Common selectable markers include antibiotic resistance, fluorescent markers, and biochemical markers. Several different antibiotic resistance genes have been used successfully for selection of microalgal transformants, including blastocidin, bleomycin (see, for example, Apt et al., 1996, supra; Fischer et al., 1999, supra; Fuhrmann et al., Plant J., 19, 353- 61, 1999, Lumbreras et al., Plant J., 14(4):441-447, 1998; Zaslayskaia et al., J. Phycol., 36:379-386, 2000), spectinomycin (Cerutti et al., Genetics, 145: 97-110, 1997; Doetsch et al., Curr. Genet., 39, 49-60, 2001; Fargo, Mol. Cell. Biol., 19:6980-90, 1999), streptomycin (Berthold et al., Protist, 153:401-412, 2002), paromomycin (Jakobiak et al., Protist, supra.; Sizova et al., Gene, 277:221-229, 2001), nourseothricin (Zaslayskaia et al., 2000, supra), G418 (Dunahay et al., 1995, supra; Poulsen and Kroger, FEBS Lett., 272:3413-3423, 2005, Zaslayskaia et al., 2000, supra), hygromycin (Berthold et al., 2002, supra), chloramphenicol (Poulsen and Kroger, 2005, supra), and many others. Additional selectable markers for use in microalgae such as Chlamydomonas can be markers that provide resistance to kanamycin and amikacin resistance (Bateman, Mol. Gen. Genet. 263:404-10, 2000), zeomycin and phleomycin (e.g., ZEOCIN™ pheomycin D1) resistance (Stevens, Mol. Gen. Genet. 251:23-30, 1996), and paramomycin and neomycin resistance (Sizova et al., 2001, supra). Other fluorescent or chromogenic markers that have been used include luciferase (Falciatore et al., J. Mar. Biotechnol., 1: 239-251, 1999; Fuhrmann et al., Plant Mol. Biol., 2004; Jarvis and Brown, Curr. Genet., 19: 317-322, 1991), β-glucuronidase (Chen et al., 2001, supra; Cheney et al., 2001, supra; Chow and Tung, 1999, supra; El-Sheekh, 1999, supra; Falciatore et al., 1999, supra; Kubler et al., J. Mar. Biotechnol., 1:165-169, 1994), β-galactosidase (Gan et al., J. Appl. Phycol., 15:345-349, 2003; Jiang et al., Plant Cell Rep., 21:1211-1216, 2003; Qin et al., High Technol. Lett., 13:87-89, 2003), and green fluorescent protein (GFP) (Cheney et al., 2001, supra; Ender et al., Plant Cell, 2002, Franklin et al., Plant 2002; 56, 148, 210).

One skilled in the art will readily appreciate that a variety of known promoter sequences can be usefully deployed for transformation systems of microalgal species in accordance with the present invention. For example, the promoters commonly used to drive transgene expression in microalgae include various versions of the of cauliflower mosaic virus promoter 35S (CaMV35S), which has been used in both dinoflagellates and chlorophyta (Chow et al, Plant Cell Rep., 18:778-780, 1999; Jarvis and Brown, Curr. Genet., 317-321, 1991; Lohuis and Miller, Plant .J., 13:427-435, 1998). The SV40 promoter from simian virus has also reported to be active in several algae (Gan et al., J. Appl. Phycol., 151 345-349, 2003; Qin et al., Hydrobiologia 398-399, 469-472, 1999). The promoters of RBCS2 (ribulose bisphosphate carboxylase, small subunit) (Fuhrmann et al., Plant J., 19:353-361, 1999) and PsaD (abundant protein of photosystem I complex; Fischer and Rochaix, FEBS Lett. 581:5555-5560, 2001) from Chlamydomonas can also be useful. The fusion promoters of HSP70A/RBCS2 and HSP70A/β2TUB (tubulin) (Schroda et al., Plant J., 21:121-131, 2000) can also be useful for an improved expression of transgenes, in which HSP70A promoter may serve as a transcriptional activator when placed upstream of other promoters. High-level expression of a gene of interest can also be achieved in, for example diatoms species, under the control of a promoter of an fcp gene encoding a diatom fucoxanthin-chlorophyll a/b binding protein (Falciatore et al., Mar. Biotechnol., 1:239-251, 1999; Zaslayskaia et al., J. Phycol. 36:379-386, 2000) or the vcp gene encoding a eustigmatophyte violaxanthin-chlorophyll a/b binding protein (see U.S. Pat. No. 8,318,482, incorporated by reference herein). If so desired, inducible promoters can provide rapid and tightly controlled expression of genes in transgenic microalgae. For example, promoter regions of the NR genes encoding nitrate reductase can be used as such inducible promoters. The NR promoter activity is typically suppressed by ammonium and induced when ammonium is replaced by nitrate (Poulsen and Kroger, FEBS Lett 272:3413-3423, 2005), thus gene expression can be switched off or on when microalgal cells are grown in the presence of ammonium/nitrate. Additional algal promoters that can find use in the constructs and transformation systems provided herein include those disclosed in U.S. Pat. No. 8,883,993; U.S. Patent Appl. Pub. No. US 2013/0023035; U.S. Patent Application Pub. No. US 2013/0323780; and U.S. Patent Application Pub. No. US 2014/0363892, all incorporated herein by reference in their entireties.

Host cells can be either untransformed cells or cells that are already transfected with at least one nucleic acid molecule. For example, an algal host cell that is engineered to have attenuated expression of a lipid regulator gene can further include one or more genes that may confer any desirable trait, such as, but not limited to, increased production of biomolecules of interest, such as one or more proteins, pigments, alcohols, or lipids.

### Type I FAS Genes

The examples provided herein demonstrate that a Type I FAS from an animal species (Danio rerio, zebrafish) and a heterokont species (a species of the genus Aurantiochytrium, a labyrinthulomycete (chytrid)) are operable in a eukaryotic algal cell. This is surprising because algae do not have Type I FAS, thus these enzymes are very different from the algal Type II FAS, which is a system of separate enzymes localized to the chloroplast. The introduced genes encode Type I FAS polypeptides that do not originate from chloroplast-containing organisms and the encoded polypeptides do not have chloroplast transit sequences that localize polypeptides to the chloroplast. Thus the FAS genes expressed in algae were not transported into the chloroplast but were functional in the cytoplasm, which is not the site of fatty acid synthesis occurs in algal cells.

Fungal Type I FAS genes commonly include two polyprotein subunits called FAS1 and FAS2, where the FAS1 subunit includes acetyl transferase (AT), enoyl reductase (ER), dehydratase (DH), malonyl/acetyl transferase (MT) domains, and the FAS2 subunit includes an acyl carrier protein (ACP), ketoacylreductase (KR), ketoacyl synthase (KS), and phosphopantetheine transferase (PPT), with the final subunit composition of the fungal Type I FAS being α₆β₆ (Schweizer and Hofmann (2004) Microbiol. Mol. Biol. Rev. 68:501-517).

Animal Type I FAS genes encode a single polydomain protein having the enzymatic domains ketoacyl synthase (KS; which may be characterized for example as conserved domain cd00833, or any of Pfam domains PF00109, PF02810, and PF16197), acyltransferase (AT, which may be characterized for example as Pfam domain PF00698 or conserved domain cl08282), dehydratase (DH, which may be characterized for example as conserved domain cl00509 or Pfam PF14765), ketoacylreductase (KR, which may be characterized for example as conserved domain cd08954), a phosphopantothiene attachment site (PP) also referred to as ACP (acyl carrier protein), and a thioesterase domain, typically in the order KS-AT-DH-ER-KR-PP-TE. The final Type I FAS has the structure a₂ (Schweizer and Hofmann (2004) Microbiol. Mol. Biol. Rev. 68:501-517).

TABLE 1
Animal Type I FAS polypeptide domains

| | Ketoacyl | Acyl- | De- | enoyl | Ketoacyl | | |
|---|---|---|---|---|---|---|---|
| | synthase | transferase | hydratase | reductase | reductase | | |
| Species | (KS) | (AT) | (DH) | (ER) | (KR) | PP | TE |
| Danio rerio | cd00833: | pfam | Pfam | smart | cd08954 | smart | cl21454: |
| SEQ ID | 2-404; | 00698: | 14765: | 00829: | 1863- | 00823 | 2245- |
| NO: 3 | pfam | 493-810 | 855-1113: | 1571- | 2118; | 2134- | 2488; |
| | 00109: | | cl00509: | 1860 | pfam | 2186; | pfam |
| | 1-239 | | 879-1070 | pfam | 08659: | pfam | 00975 |
| | Pfam | | (hot dog | 00107: | 1892- | 00550 | 2245-2501 |
| | 02801 | | fold) | 1685- | 2071 | | |
| | 243-360; | | | 1822 | | | |
| | pfam | | | | | | |
| | 16197: | | | | | | |
| | 362-472 | | | | | | |
| Aquila | cd00833: | pfam | Pfam | smart | cd08954: | smart | pfam |
| chrysaetos | 2-404; | 00698: | 14765: | 00829: | 1880- | 00823 | 00975 |
| SEQ ID | pfam | 492-809 | 866-1111; | 1568-1858; | 2116; | 2115- | 2245- |
| NO: 20 | 00109; | | cl00509: | pfam | pfam | 2183; | 2504; |
| | 1-239 | | 874-1060 | 00107: | 08659: | pfam | pfam |
| | Pfam | | (hot dog | 1683-1819 | 1889- | 00550: | 00975 |
| | 02801 | | fold) | | 2069 | 2129- | 2245-2504 |
| | 243-360; | | | | | 2196 | |
| | pfam | | | | | | |
| | 16197: | | | | | | |
| | 362-471 | | | | | | |
| Drosophila | pfam | pfam | Pfam | pfam | pfam | pfam | pfam |
| Melanogaster | 00109: | 00698: | 14765: | 00107: | 08659: | 00550: | 00975 |
| SEQ ID | 37-277 | 531-848 | 882-1132; | 1606-1739 | 1812- | 2050- | 2183-2436 |
| NO: 22 | Pfam | | | | 1992 | 2115 | |
| | 02801 | | | | | | |
| | 281-398; | | | | | | |
| | pfam | | | | | | |
| | 16197: | | | | | | |
| | 400-509 | | | | | | |
| Rattus Norvegicus | cd00833: | cl08282 | cl00509; | smart | cd08954: | smart | cl21454: |
| SEQ ID | 38-442; | 531-848; | 888-1132 | 00829: | 1799- | 00823 | 2184- |
| NO: 24 | pfam | pfam | (hot dog | 1491- | 2039; | 2052- | 2433; |
| | 00109: | 00698: | fold); | 1781; | pfam | 2104; | pfam |
| | 1-239 | 493-810 | Pfam | pfam | 08659: | pfam | 00975 |
| | Pfam | | 14765: | 00107: | 1879- | 00550: | 2236-2495 |
| | 02801 | | 854-1107 | 1673- | 2059 | 2120- | |
| | 243-360; | | | 1809 | | 2187 | |
| | pfam | | | | | | |
| | 16197 | | | | | | |
| | 362-472 | | | | | | |

TABLE 2
Labyrinthylomycete Type I FAS polypeptide domains

| | | | | Malonyl | Keto- | Keto- | |
|---|---|---|---|---|---|---|---|
| | Acyl- | enoyl | | trans- | acyl- | acyl | |
| | transf | reduct | Dehydratase | acylase | reductase | synthase | |
| Species | (AT) | (ER) | (DH) | (MT) | (KR) | (KS) | PPT |
| Aurantiochytrium | cl08282: | COG | cl00509: | COG | cd08950: | cd00828: | cl00500: |
| sp. | 38-407; | 4981: | 1460-1584 | 0331: | 2901- | 3371- | 4029- |
| SEQ ID | Pfam | 465-1220 | (hot dog | 1583- | 3161; | 3874; | 4124; |
| NO: 8 | 16073: | | fold); | 2023; | pfam | COG | Pfam |
| | | | pfam | Pfam | 00106 | 0304 | 01648: |
| | | | 01575: | 00698: | 2908- | 3411- | 4020- |
| | | | 1451-1572 | 1587- | 3114 | 3878; | 4144; |
| | | | | 1954 | | Pfam | COG0736: |
| | | | | | | 00109: | 4041-4146 |
| | | | | | | 3403- | |
| | | | | | | 3630; | |
| | | | | | | Pfam | |
| | | | | | | 02801 | |
| | | | | | | 3728- | |
| | | | | | | 3831; | |
| Schizochytrium | cl00509: | COG | cl00509: | COG | cd08950: | cd00828: | cl00500: |
| sp. | 45-417; | 4981: | 1456-1579 | 0331: | 2889- | 3379- | 4029- |
| SEQ ID | Pfam | 466-1219 | (hot dog | 1578- | 3149; | 3863; | 4124; |
| NO: 19 | 16073: | | fold); | 2018; | pfam | COG | Pfam |
| | 44-262 | | pfam | Pfam | 00106 | 0304 | 01648: |
| | | | 01575: | 00698: | 2896- | 3399- | 4008-4132 |
| | | | 1447-1567 | 1582- | 3102 | 3867; | |
| | | | | 1949 | | Pfam | |
| | | | | | | 00109: | |
| | | | | | | 3369- | |
| | | | | | | 3618; | |
| | | | | | | Pfam | |
| | | | | | | 02801 | |
| | | | | | | 3718- | |
| | | | | | | 3820; | |

Table 1 provides the localization of domains in animal FAS polypeptides as characterized by pfam (protein sequences are searchable for pfam domain at pfam.xfam.org) and by conserved domains as searched at the national center for biotechnology information website using the “CD” BLAST search function (ncbi.nlm.nih.gov/Structure/cdd/wrpsb.cgi). Such searches can be performed to identify potential Type I FAS genes and to determine conserved functional regions of Type I FAS genes, i.e., domains less tolerant of sequence variation.

The Danio rerio Type I FAS (SEQ ID NO:3) is 70% identical to the Type I FAS of the golden eagle, Aquila chrysaetos (SEQ ID NO:20), 69% identical to the Type I FAS of the rat, Rattus norvegicus (SEQ ID NO:24), and 49% identical to the Type I FAS of the fruit fly, Drosophila melanogaster (SEQ ID NO:22). Although the Danio rerio Type I FAS (SEQ ID NO:3) has no significant homology to the Type I FAS of the labyrinthulomycete Aurantiochytrium (SEQ ID NO:8), both the Danio rerio Type I FAS (SEQ ID NO:3) and the Type I FAS of the labyrinthulomycete Aurantiochytrium (SEQ ID NO:8) were functional in algal cells, indicating a wide range of Type I FAS genes are functional in algal cells.

Phosphopantetheine transferase is typically a separate enzyme encoded by a separate gene in animal cells. In preferred embodiments the transgenic alga that includes an exogenous Type I FAS gene encoding a Type I FAS derived from an animal species further includes an exogenous gene encoding a L-annnoadipate-semialdehyde dehydrogenase-phosphopantetheinyl transferase, or simply a “pantetheine phosphotransferase” or “PPT”. The PPT gene can encode, for example, a PPT that is at least 75%, at least 80%, at least 85%, at least 90%, at least 95% identical to a naturally-occurring PPT. In some embodiments where the alga includes a transgene encoding a Type I FAS of animal origin (or Type I FAS derived from a Type I FAS of an animal species), the alga also preferably includes an exogenous PPT gene, which can optionally be derived from the same species as the Type I FAS. In various examples the PPT gene can encode a PPT having at least 75%, at least 80%, at least 85%, at least 90%, at least 95% identity to SEQ ID NO:6, SEQ ID NO:21, SEQ ID NO:23, or SEQ ID NO:25.

Labyrinthulomycete Type I FAS as disclosed herein (see Table 2) includes the domains acyltransferase (AT), enoyl reductase (ER), dehydratase (DH), malonyl/acetyl transferase (MT), ketoacylreductase (KR), ketoacyl synthase (KS), and phosphopantetheine transferase (PPT). The Schizochytrium Type I FAS (SEQ ID NO:19) and Aurantiochytrium Type I FAS (SEQ ID NO:8) share 80% amino acid sequence identity.

It is envisioned that a wide range of Type I FAS genes can be utilized for increasing lipid production in algae, for example, of any animal, fungal, or heterokont species. As demonstrated herein, a Type I FAS gene can be codon optimized for expression in the host alga. Penetrance of expression of the heterologous FAS gene in a transformant culture can be assessed to select strains with optimal phenotypes. Additional examples of a Type I FAS that can be encoded by a nucleic acid molecule introduced into an algal host cell include a Type I FAS from golden eagle (SEQ ID NO:20), rat (SEQ ID NO:24), and Drosophila (SEQ ID NO:22).

Although eukaryotic algae do not naturally have a Type I FAS, and the FAS of eukaryotic algae is active only in the chloroplast, the examples herein demonstrate that to increase the rate of fatty acid synthesis in an algal cell, a heterologous FAS need not be directed to the chloroplast. Without wishing to be limited to any specific mechanism, the inventors contemplate that a heterologous FAS may be more active and/or a recombinant alga may produce more lipid, when the FAS is produced and remains in the cytoplasm, where chloroplastic (negative) feedback mechanisms that may directly or indirectly downregulate FAS activity may be avoided. Thus in some exemplary embodiments, a heterologous FAS gene does not include a chloroplast localization sequence and is not designed to target the FAS to the chloroplast. In various embodiments, an exogenous FAS produced by a recombinant alga as provided herein is active in the cytoplasm of the algal cell.

Considered herein for expression in an algal cell are genes encoding Type I FAS from fungi, animals, and labyrinthuylomycetes. Provided herein are algal cells expressing genes encoding FASI polypeptides having at least 85%, at least 90%, at least 95%, at least 96%, at least 97%, at least 98% or at least 99% identity to naturally occurring Type I FAS polypeptides of animal cells. Based on information provided in the art and in the present application, one of skill could easily find regions of the Type I FAS polypeptides tolerant of mutation (i.e., outside of conserved domains and domains identified as conferring activity). Further considered herein are recombinant algae that include exogenous Type I FAS genes encoding polypeptides having at least 65%, at least 70%, at least 75%, at least 80%, at least 85%, at least 90%, at least 95%, at least 96%, at least 97%, at least 98% or at least 99% identity to the animal Type I FAS of SEQ ID NO:3, SEQ ID NO:20, SEQ ID NO:22, orSEQ ID NO:24. For example, a recombinant alga as provided herein can include an exogenous gene that encodes a Type I FAS having at least 85%, at least 90%, at least 95%, at least 96%, at least 97%, at least 98% or at least 99% identity to any of SEQ ID NO:3, SEQ ID NO:20, SEQ ID NO:22, and SEQ ID NO:24. In exemplary embodiments, a recombinant alga as provided herein can include an exogenous gene that encodes a Type I FAS having at least 85%, at least 90%, at least 95%, at least 96%, at least 97%, at least 98% or at least 99% identity to SEQ ID NO:3. The recombinant alga according to any of the embodiments can produce fatty acids or a lipid such as a fatty acid, diglyceride, or triacylglyceride, at a higher rate than a control alga.

In further embodiments, provided herein are recombinant algae that include exogenous Type I FAS genes encoding polypeptides having at least 65%, at least 70%, at least 75%, at least 80%, at least 85%, at least 90%, at least 95%, at least 96%, at least 97%, at least 98% or at least 99% identity to the labyrinthulomycete Type I FAS of SEQ ID NO:8 or SEQ ID NO:19. For example, a recombinant alga as provided herein can include an exogenous gene that encodes a Type I FAS having at least 85%, at least 90%, at least 95%, at least 96%, at least 97%, at least 98% or at least 99% identity to SEQ ID NO:8 or SEQ ID NO:19. In exemplary embodiments, a recombinant alga as provided herein can include an exogenous gene that encodes a Type I FAS having at least 85%, at least 90%, at least 95%, at least 96%, at least 97%, at least 98% or at least 99% identity to SEQ ID NO:8. The recombinant alga according to any of the embodiments can produce fatty acids or a lipid such as a fatty acid, diglyceride, or triacylglyceride, at a higher rate than a control alga.

### Methods of Producing Lipids

Also provided herein are methods of producing lipid by culturing a recombinant alga as provided herein that includes an exogenous gene encoding a Type I FAS. The methods include culturing a recombinant alga as provided herein in a suitable medium to produce lipid and recovering biomass or at least one lipid from the culture. The recombinant alga has increased lipid productivity with respect to a control alga that does not include an exogenous gene encoding a Type I FAS, for example, the recombinant alga that includes an exogenous gene encoding a Type I FAS can have at least 5%, at least 10%, at least 15%, at least 20%, at least 35%, at least 30%, at least 35%, at least 40%, at least 45%, at least 50%, at least 60%, at least 70%, at least 80%, at least 90%, or at least 100% higher fatty acid productivity, for example, hourly or daily productivity, than a control alga cultured under substantially identical conditions. The culture can be a photoautotrophic culture or mixotrophic culture, where the algae are grown in the light but are provided with a supplemental reduced carbon source, such as for example, a sugar such as glucose or an organic acid such as acetate. Culturing can be in batch, semi-continuous, or continuous mode.

The recombinant algae in some examples can be cultured in a medium that comprises ammonium, nitrate, urea, or any other suitable nitrogen source. In various examples, the medium is nitrogen replete and supports growth of the microorganism. In other embodiments, the medium is nitrogen-limited, for example includes less than 2 mM or less than 1 mM of a nitrogen source such as nitrate, urea, or ammonium, or lacks a nitrogen source.

The recombinant algae may be cultured in any suitable vessel(s), including flasks or bioreactors. In some examples, the mutant microorganism is an alga and is exposed to light for at least a portion of the culture period, in which the algae may be exposed to artificial or natural light (or natural light supplemented with artificial light). The culture comprising recombinant algae that are deregulated in their response to low light may be cultured on a light/dark cycle that may be, for example, a natural or programmed light/dark cycle, and as illustrative examples, may provide twelve hours of light to twelve hours of darkness, fourteen hours of light to ten hours of darkness, sixteen hours of light to eight hours of darkness, etc. Alternatively, algae expressing an exogenous Type I FAS gene can be cultured in continuous light.

Culturing refers to the intentional fostering of growth (e.g., increases in cell size, cellular contents, and/or cellular activity) and/or propagation (e.g., increases in cell numbers via mitosis) of one or more cells by use of selected and/or controlled conditions. The combination of both growth and propagation may be termed proliferation. A microorganism as provided herein may be cultured for at least five, at least six, at least seven at least eight, at least nine, at least ten, at least eleven at least twelve, at least thirteen, at least fourteen, or at least fifteen days, or at least one, two three, four, five, six, seven, eight, nine, or ten weeks, or longer. The culturing can be in a culture medium that is nutrient replete with respect to a control alga.

Non-limiting examples of selected and/or controlled conditions that can be used for culturing the recombinant algal microorganism can include the use of a defined medium (with known characteristics such as pH, ionic strength, and/or carbon source), specified temperature, oxygen tension, carbon dioxide levels, growth in a bioreactor, or the like, or combinations thereof In some embodiments, the microorganism or host cell can be grown mixotrophically, using both light and a reduced carbon source. Algal reduced carbon sources can include, without limitation, organic acids or their anions (e.g., acetic acid or acetate) and sugars such as glucose or fructose.

Alternatively, the algal cell can be cultured phototrophically. When growing phototrophically, the algal strain uses light as an energy source. An inorganic carbon source, such as CO₂ or bicarbonate can be used for synthesis of biomolecules by the microorganism. “Inorganic carbon”, as used herein, includes carbon-containing compounds or molecules that cannot be used as a sustainable energy source by an organism. Typically “inorganic carbon” can be in the form of CO₂ (carbon dioxide), carbonic acid, bicarbonate salts, carbonate salts, hydrogen carbonate salts, or the like, or combinations thereof, which cannot be further oxidized for sustainable energy nor used as a source of reducing power by organisms. A microorganism grown photoautotrophically can be grown on a culture medium in which inorganic carbon is substantially the sole source of carbon. For example, in a culture in which inorganic carbon is substantially the sole source of carbon, any organic (reduced) carbon molecule or organic carbon compound that may be provided in the culture medium either cannot be taken up and/or metabolized by the cell for energy and/or is not present in an amount sufficient to provide sustainable energy for the growth and proliferation of the cell culture.

Algal microorganisms that can be useful in accordance with the methods of the present invention can be found in various locations and environments throughout the world. The particular growth medium for optimal propagation and generation of lipid and/or other products can vary and may be optimized to promote growth, propagation, or production of a product such as a lipid, protein, pigment, antioxidant, etc. In some cases, certain strains of microorganisms may be unable to grow in a particular growth medium because of the presence of some inhibitory component or the absence of some essential nutritional requirement of the particular strain of microorganism or host cell.

Solid and liquid growth media are generally available from a wide variety of sources, as are instructions for the preparation of particular media suitable for a wide variety of strains of microorganisms. For example, various fresh water and salt water media can include those described in Barsanti (2005) Algae: Anatomy, Biochemistry & Biotechnology, CRC Press for media and methods for culturing algae. Algal media recipes can also be found at the websites of various algal culture collections, including, as nonlimiting examples, the UTEX Culture Collection of Algae (sbs.utexas.edu/utex/media.aspx); Culture Collection of Algae and Protozoa (ccap.ac.uk); and Katedra Botaniky (botany.natur.cuni.cz/algo/caup-media.html).

The culture methods can optionally include inducing expression of one or more genes and/or regulating a metabolic pathway in the microorganism. Inducing expression can include adding a nutrient or compound to the culture, removing one or more components from the culture medium, increasing or decreasing light and/or temperature, and/or other manipulations that promote expression of the gene of interest. Such manipulations can largely depend on the nature of the (heterologous) promoter operably linked to the gene of interest.

In some embodiments of the present invention, the algal microorganisms having increased lipid productivity can be cultured in a photobioreactor equipped with an artificial light source, and/or having one or more walls that is transparent enough to light, including sunlight, to enable, facilitate, and/or maintain acceptable algal growth and proliferation. For production of fatty acid products or triglycerides, photosynthetic microorganisms or host cells can additionally or alternately be cultured in shake flasks, test tubes, vials, microtiter dishes, petri dishes, or the like, or combinations thereof.

Additionally or alternately, recombinant photosynthetic microorganisms or host cells may be grown in ponds, canals, sea-based growth containers, trenches, raceways, channels, or the like, or combinations thereof. In such systems, the temperature may be unregulated, or various heating or cooling method or devices may be employed As with standard bioreactors, a source of inorganic carbon (such as, but not limited to, CO₂, bicarbonate, carbonate salts, and the like), including, but not limited to, air, CO₂-enriched air, flue gas, or the like, or combinations thereof, can be supplied to the culture. When supplying flue gas and/or other sources of inorganic that may contain CO in addition to CO₂, it may be necessary to pre-treat such sources such that the CO level introduced into the (photo)bioreactor do not constitute a dangerous and/or lethal dose with respect to the growth, proliferation, and/or survival of the microorganisms.

The recombinant algal microorganisms can optionally include one or more non-native genes encoding a polypeptide for the production of a product, such as but not limited to a lipid.

The methods include culturing a recombinant microorganism as provided herein, such as a mutant microorganism as provided herein that has increased lipid productivity with respect to a control cell while producing at least 50% of the biomass produced by a control cell under the same culture conditions to produce biomass or lipid. Lipids can be recovered from culture by recovery means known to those of ordinary skill in the art, such as by whole culture extraction, for example, using organic solvents or by first isolating biomass from which lipids are extracted (see, for example, Hussein et al. Appl. Biochem. Biotechnol. 175:3048-3057; Grima et al. (2003) Biotechnol. Advances 20:491-515). In some cases, recovery of fatty acid products can be enhanced by homogenization of the cells (Gunerken et al. (2015) Biotechnol. Advances 33:243-260). For example, lipids such as fatty acids, fatty acid derivatives, and/or triglycerides can be isolated from algae by extraction of the algae with a solvent at elevated temperature and/or pressure, as described in the co-pending, commonly-assigned U.S. patent publication No. US 2013/0225846 entitled “Solvent Extraction of Products from Algae”, filed on Feb. 29, 2012, which is incorporated herein by reference in its entirety.

Biomass can be harvested, for example, by centrifugation or filtering. The biomass may be dried and/or frozen. Further products may be isolated from biomass, such as, for example, various lipids or one or more proteins. Also included in the invention is an algal biomass comprising biomass of lipid regulator mutant, such as any disclosed herein, such as but not limited to a lipid regulator mutant that includes a mutation in a gene encoding a polypeptide having at least 50%, at least 55%, at least 60%, at least 65%, at least 70%, at least 75%, at least 80%, at least 85%, at least 90%, or at least 95% identity to a polypeptide comprising an amino acid sequence selected from the group consisting of SEQ ID NO:3 and SEQ ID NO:8.

Alternatively or in addition to any of the forgoing embodiments, the invention provides the following embodiments:

Embodiment 1 is a recombinant alga that comprises an exogenous gene encoding a Type I FAS, optionally wherein one or more of the following are fulfilled:

The Type I FAS has at least 85%, at least 90%, at least 95%, at least 96%, at least 97%, at least 98%, at least 99%, or about 100% sequence identity to a naturally-occurring Type I FAS;

the Type I FAS does not include a chloroplast transit sequence;

the Type I FAS is derived from an animal, fungal, or labyrinthulomycete FAS; and

the recombinant alga further comprises an exogenous gene encoding a PPT.

Embodiment 2 is a recombinant alga according to embodiment 1, wherein

the Type I FAS is derived from a Type I FAS of an animal, such as a mammal, bird, fish, reptile, amphibian, marsupial, insect, arachnid, mollusk, or crustacean;

optionally wherein the Type I FAS has at least 50%, at least 55%, at least 60%, at least 65%, at least 70%, at least 75%, at least 80%, or at least 85%, for example at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity with a Type I FAS selected from the group consisting of SEQ ID NO:3, SEQ ID NO:20, SEQ ID NO:22, and SEQ ID NO:24.

Embodiment 3 A recombinant alga according to embodiment 2, wherein

the recombinant alga further comprises an exogenous nucleic acid sequence that encodes a pantetheine phosphotransferase (PPT), optionally wherein the PPT is a derived from the PPT of a mammal, bird, fish, reptile, amphibian, marsupial, insect, arachnid, mollusk, or crustacean, optionally wherein the PPT has at least 50%, at least 55%, of at least 70%, at least 65%, at least 70%, at least 75%, at least 80%, or at least 85%, for example at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to a PPT selected from the group consisting of SEQ ID NO:6, SEQ ID NO:21, SEQ ID NO:23, and SEQ ID NO:25.

Embodiment 4 is a recombinant alga according to embodiment 1, wherein

the Type I FAS is derived from a Type I FAS of a labyrintulomycete, optonally wherein the the Type I FAS is derived from a Type I FAS of a Labryinthula, Labryinthuloides, Thraustochytrium, Schizochytrium, Aplanochytrium, Aurantiochytrium, Oblongichytrium, Japonochytrium, Diplophrys, or Ulkenia species;

optionally wherein the Type I FAS has at least 50%, at least 55%, at least 60%, at least 65%, at least 70%, at least 75%, at least 80%, or at least 85%, for example at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity SEQ ID NO:8 or SEQ ID NO:19.

Embodiment 5 is a recombinant alga according to any of embodiments 1-4, wherein the recombinant alga further comprises:

one or more exogenous genes encoding a gene for making fatty acid or fatty acid derivative, otionally wherein the fatty acid derivative is a fatty aldehyde, a fatty alcohol, a fatty acid ester, a wax ester, an alkane, or an alkene; or

one or more genes for producing a diacylglyceride or triacylglyceride.

Embodiment 6 is a recombinant alga according to embodiment 5, wherein the recombinant alga further comprises one or more exogenous genes encoding a 4-hydroxybenzoyl-CoA thioesterase, acyl-ACP thioesterase, acyl-CoA synthetase, acyl-CoA reductase, carboxylic acid reductase, acyl-ACP reductase, fatty aldehyde reductase, wax synthase, fatty acid decarboxylase, fatty aldehyde decarbonylase, acetyl-CoA carboxylase, malonyl CoA: ACP transacylase, beta-ketoacyl-ACP synthase, acyl-ACP thioesterase, acyl-CoA thioesterase, a polypeptide having lipolytic activity, glycerolphosphate acyltransferase (GPAT), lysophosphatidic acid acyltransferase (LPAAT), a phosphatidic acid phosphatase (PA), or diacylglycerol O-acyltransferase (DGAT).

Embodiment 7 is a recombinant alga according to any of embodiments 1-6, wherein the fatty acid productivity of the recombinant alga is at least 5%, at least 10%, at least 15%, at least 20%, at least 35%, at least 30%, at least 35%, at least 40%, at least 45%, at least 50%, at least 60%, at least 70%, at least 80%, at least 90%, or at least 100% higher that the fatty acid productivity by a control microorganism cultured under substantially identical conditions, optionally wherein any one or more of the following are fulfilled:

(a) the control microorganism is a wild type microorganism;

(b) the culture medium is nitrogen replete;

(c) the culture conditions are mixotrophic

(d) the culture conditions are phototrophic;

(e) the alga is a microagla, optionally of a genus selected from the group consisting of Achnanthes, Amphiprora, Amphora, Ankistrodesmus, Asteromonas, Boekelovia, Bolidomonas, Borodinella, Botrydium, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Desmodesmus, Dunaliella, Elipsoidon, Emiliania, Eremosphaera, Ernodesmius, Euglena, Eustigmatos, Franceia, Fragilaria, Fragilaropsis, Gloeothamnion, Haematococcus, Hantzschia, Heterosigma, Hymenomonas, Isochrysis, Lepocinclis, Micractinium, Monodus, Monoraphidium, Nannochloris, Nannochloropsis, Navicula, Neochloris, Nephrochloris, Nephroselmis, Nitzschia, Ochromonas, Oedogonium, Oocystis, Ostreococcus, Parachlorella, Parietochloris, Pascheria, Pavlova, Pelagomonas, Phceodactylum, Phagus, Picochlorum, Platymonas, Pleurochrysis, Pleurococcus, Prototheca, Pseudochlorella, Pseudoneochloris, Pseudostaurastrum, Pyramimonas, Pyrobotrys, Scenedesmus, Schizochlamydella, Skeletonema, Spyrogyra, Stichococcus, Tetrachlorella, Tetraselmis, Thalassiosira, Tribonema, Vaucheria, Viridiella, Vischeria, and Volvox.

Embodiment 8 is a method of producing a lipid comprising culturing a recombinant alga according to any of embodiments 1-7 to produce a lipid; optionally further comprising recovering the lipid from the cells, culture medium, or whole culture.Embodiment 9 is a method according to embodiment 8, wherein any one or more of the following are fulfilled:

fatty acid productivity is measured as the rate of fatty acid production;

the culturing is under nutrient replete conditions;

the culturing is under nitrogen replete conditions;

the culturing is under mixotrophic conditions;

the culturing is under photoautotrophic conditions; and

the culture is a batch, semi-continuous, or continuous culture.

Embodiment 9 is a method according to embodiment 7 or 8, wherein the lipid comprises a fatty acid, a fatty acid derivative, a diacylglyceride, or a triacylglyceride.

Embodiment 10 is method for producing lipid according to embodiment 8 or 9 wherein the mutant produces at least 5% more lipid, for example at least 5%, at least 10%, at least 15%, at least 20%, at least 35%, at least 30%, at least 35%, at least 40%, at least 45%, at least 50%, at least 60%, at least 70%, at least 80%, at least 90%, or at least 100% more FAME lipid, during a culture period of from at least one hour to three days or less.

### EXAMPLES

### Media Used in Examples

PM074 is a nitrogen replete medium that is 10× F/2 made by adding 1.3 ml PROLINE® F/2 Algae Feed Part A (Aquatic Eco-Systems) and 1.3 ml PROLINE® F/2 Algae Feed Part B (Aquatic Eco-Systems) to a final volume of 1 liter of a solution of Instant Ocean salts (35 g/L) (Aquatic Eco Systems, Apopka, FL). Proline A and Proline B together include 8.8 mM NaNO₃, 0.361mM NaH₂PO₄·H₂O, 10× F/2 Trace metals, and 10X F/2 Vitamins (Guillard (1975) Culture of phytoplankton for feeding marine invertebrates. in “Culture of Marine Invertebrate Animals.” (eds: Smith W. L. and Chanley M. H.) Plenum Press, New York, USA. pp 26-60).

### Example 1

### Cloning of Type I FAS Genes

The codon optimized open reading frames (SEQ ID NOs:2, 5, and 7) were assembled in vectors harboring promoters and terminators flanking the type I FAS pathway genes to drive their expression in Nannochloropsis gaditana. The “DrFAS” pathway, sourced from zebrafish (Danio rerio) consists of two genes: DrFAS (coding sequence provided as SEQ ID NO:1), encoding the catalytic type I FAS protein (SEQ ID NO:3), and DrPPT (coding sequence provided as SEQ ID NO:4), encoding a pantetheine phosphotransferase (PPT; SEQ ID NO:6), which is required for activating the ACP domain of the DrFAS protein. The “ChytFAS” pathway, sourced from a proprietary labyrinthylomycete strain, consists of a single ChytFAS gene encoding a protein (SEQ ID NO:8) which harbors both Type I FAS and PPT activity in one polypeptide. These vectors were used to generate transformants in Nannochloropsis (transformation was essentially according to US 2015/0183838, incorporated by reference) which were screened for individual lines in which the type I FAS genes were expressed, as determined by western blotting.

### Example 2

### Expression of Heterologous Type I FAS Genes in Nannochloropsis gaditana

Nucleic acid sequences encoding the zebrafish Danio rerio Type I Fatty Acid Synthase (Type 1 FAS) (SEQ ID NO:3) and encoding a Type I FAS of a proprietary isolated labyrinthuylomycete strain of the Aurantiochytrium genus (SEQ ID NO:8) were cloned into constructs designed for expression of the genes in the Eustigmatophyte alga Nannochloropsis gaditana, allowing isolation of strains demonstrating the functionality of heterologous Type I FAS enzymes in the cytoplasm of an alga for the first time.

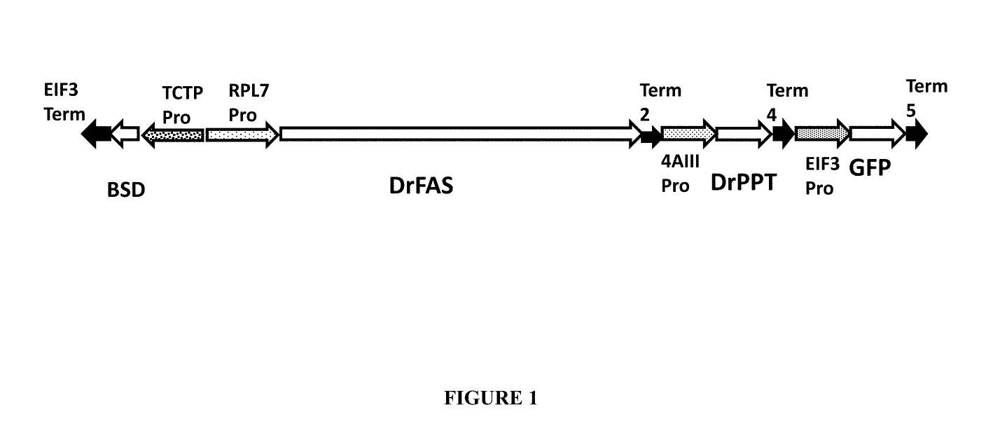

The construct for expression of C. rerio Type I FAS, pSGE-6200 (FIG. 1), included the gene encoding the D. rerio Type I FAS, termed “DrFAS”, which was codon optimized for N. gaditana (SEQ ID NO:2) and operably linked to the N. gaditana RPL7 promoter (SEQ ID NO:9), positioned 5′ of the DrFAS coding sequence, and the N. gaditana ‘Terminator 2’ sequence (SEQ ID NO:10), positioned at the 3′ end of the DrFAS coding sequence (SEQ ID NO:2). The expression construct also included a nucleic acid sequence (SEQ ID NO:5) encoding the D. rerio pantetheine phosphotransferase (L-aminoadipate-semialdehyde dehydrogenase-phosphopantetheinyi transferase or PPT; SEQ ID NO:6) which is required for activating the ACP domain of the DrFAS protein. The PPT gene (SEQ ID NO:5) used in the construct was also codon-optimized for N. gaditana and was operably linked at its 5′ end to the N. gaditana 4AIII promoter (SEQ ID NO:11), and at its 3′ end to N. gaditana Terminator 4 (SEQ ID NO:12). Upstream of the DrFAS and PPT genes was a cassette for the expression of the codon-optimized “blast” or “BSD” gene (SEQ ID NO:13) operably linked to the TCTP promoter (SEQ ID NO:14) at its 5′ end (oriented in a direction opposite to the RPL7 promoter (SEQ ID NO:9) positioned to drive expression of the DrFAS gene), and to the EIF3 terminator (SEQ ID NO:15) at its 3′ end. Downstream of the DrFAS and PPT genes was a cassette for GFP expression in which the coding sequence for TurboGFP (codon optimized for N. gaditana, SEQ ID NO:16) was operably linked to EIF3 promoter (SEQ ID NO:17) and N. gaditana terminator 5 (SEQ ID NO:18). The GFP expression cassette was oriented in the same 5′ to 3′ direction as the DrFAS and PPT genes.

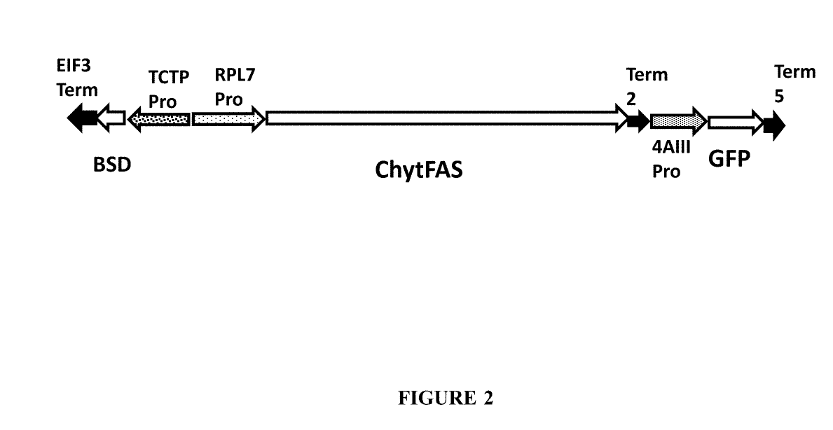

The construct for expression of the labyrinthulomycete (“chytrid”) Type I FAS, pSGE-6167 (FIG. 2), included the gene encoding the Aurantiochytrium Type I FAS (SEQ ID NO:8), termed “ChytFAS”, codon optimized for N. gaditana (SEQ ID NO:7) operably linked to the N. gaditana RPL7 promoter (SEQ ID NO:9) 5′ of the ChytFAS coding sequence, and the N. gaditana ‘Terminator 2’ sequence (SEQ ID NO:10) at the 3′ end of the DrFAS coding sequence. This construct did not include a separate PPT gene, as the Chytrid FAS includes that enzymatic activity. Upstream of the ChytFAS gene was the same blast expression cassette as provided in the DrFAS construct, also oriented such that the direction of transcription was opposite that of the FAS gene, and downstream of the ChytFAS gene was the same GFP expression cassette that was employed in the DrFAS construct, again oriented in the same direction as the FAS gene.

DNA fragments that included these expression cassettes of DrFAS expression construct pSGE-6200 and ChytFAS construct pSGE-6167 were transformed, separately, as linear molecules (with the vector backbone removed) into Nannochloropsis by electroporation essentially as described in US 2014/0220638, incorporated herein by reference. Transformants were selected on plates that contained blastocidin and screened for the presence of the construct by PCR.

Clones that included the construct were then screened for penetrance by flow cytometry monitoring for GFP fluorescence and selecting tranformed lines that demonstrated a sinble fluorescence peak shifted to the right (i.e., to a higher fluorescence level) as compared with the autofluorescence peak of wild type cells subjected to flow cytometry, as described in commonly-owned co-pending U.S. Patent application serial number 14/986,492, filed Dec. 31, 2015 and corresponding PCT application PCT/US15/068356 published as WO 2016/109840, as well as U.S. Patent Application Publication U.S. 2016/0304896, all of which are incorporated herein by reference in their entireties. Six selected “DrFAS” expression construct transformants were found to have complete penetrance, as the transformants displayed a single fluorescence peak that was shifted to a higher fluorescence level with respect to the wild type fluorescence peak.

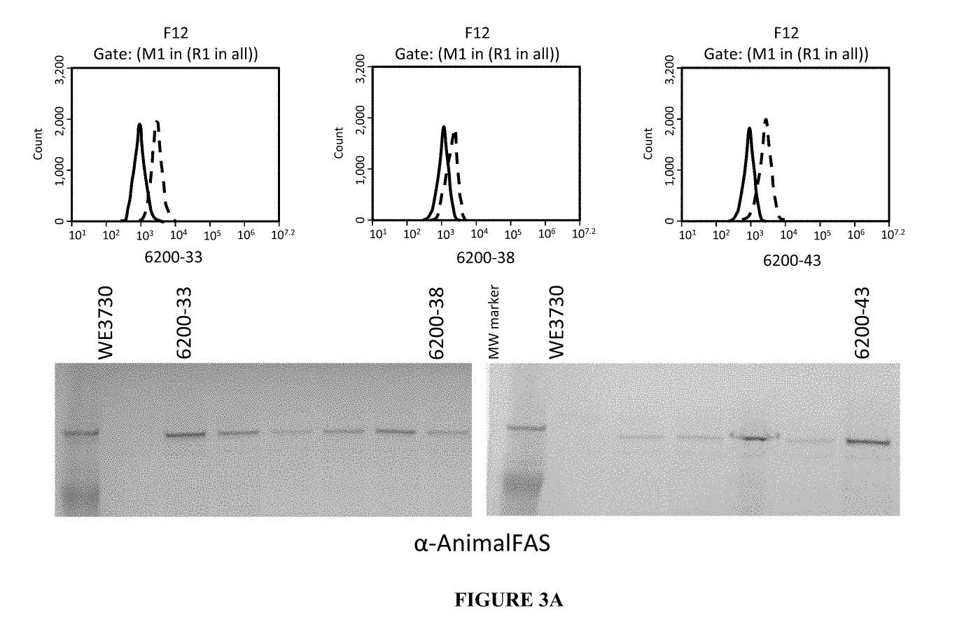

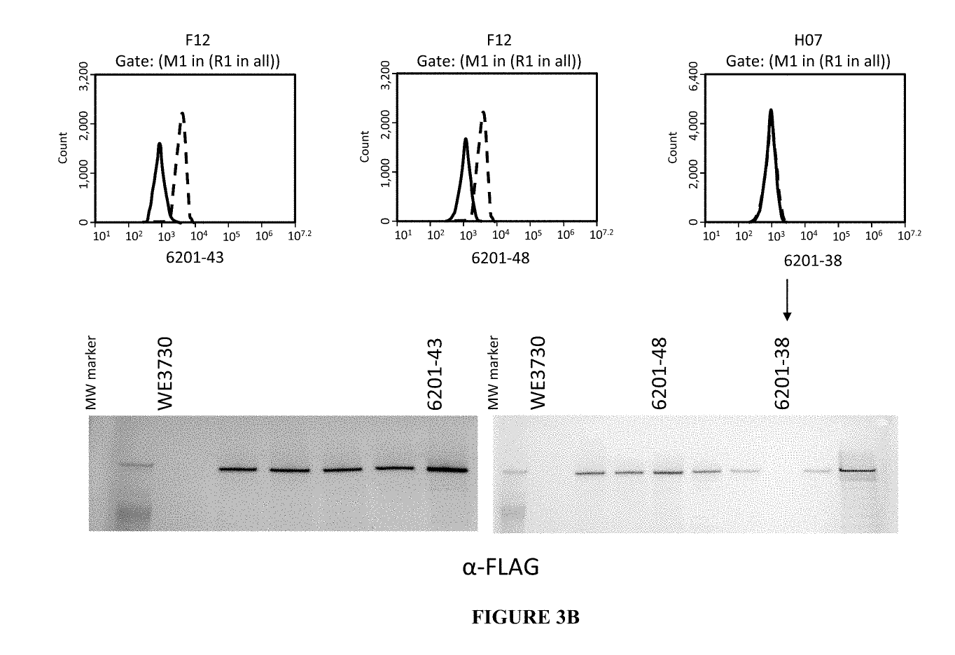

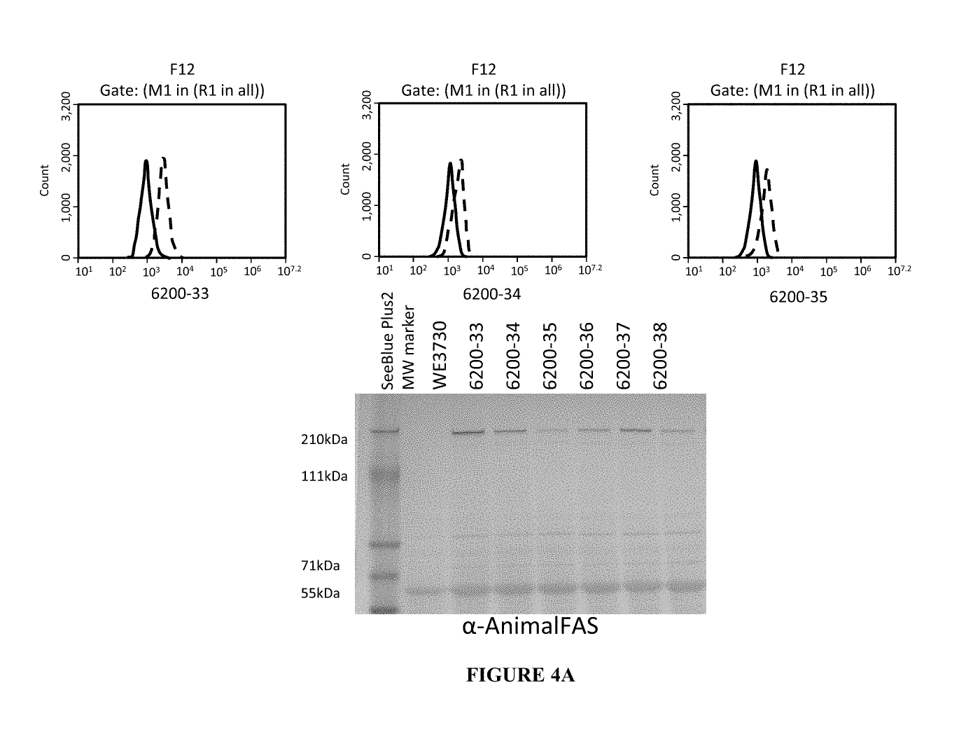

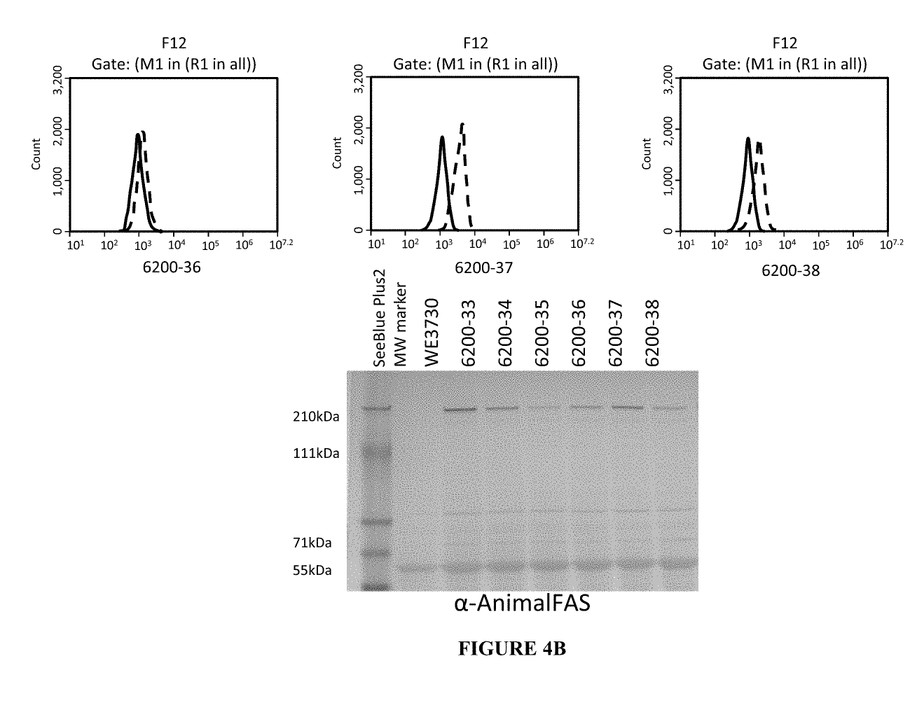

The selected transformants were also tested for FAS protein expression by Western blot using an antibody reactive against animal Type I FAS or a FLAG tag present in the Dr FAS gene in the 6201 construct used to make some of the DrFAS transformants (FIGS. 3A and 3B). In FIGS. 3A and 3B, Western blots are shown in which it can be seen that each fully penetrant clone also demonstrated protein expression. (In contrast strain 6201-38 in FIG. 3B did not demonstrate a peak higher than the wild type peak in flow cytometry, i.e., did not demonstrate penetrant expression and did not show any protein expression in the Western blot.) Unlabeled lanes on the gel show protein reactivity of clones that were not determined to be fully penetrant (i.e., they displayed more than one peak, one of which coincided with wild-type, or background, fluorescence, or they displayed a single peak that was coincident with the wild type or background peak). Thus, screening for protein level alone did not result in the identification of fully penetrant lines (expression throughout the culture). FIG. 4A provides the flow cytometry traces of six DrFAS lines that demonstrated complete penetrance and FIG. 4B provides the Western blots of these lines with anti-animal FAS antibody. Interestingly, for these fully penetrant DrFAS lines, protein level as assessed by Western signal intensity corresponds to the degree of separation of the transformant peak from the background (wild type) peak; for example, strains 6200-33 (FIG. 4A) and 6200-37 (FIG. 4B) have the most intense Western bands and the greatest separation of their flow cytometry fluorescence peaks from the wild type fluorescence peak, indicating that the degree of GFP gene expression is reflected in the degree of expression of the linked gene in these cultures.

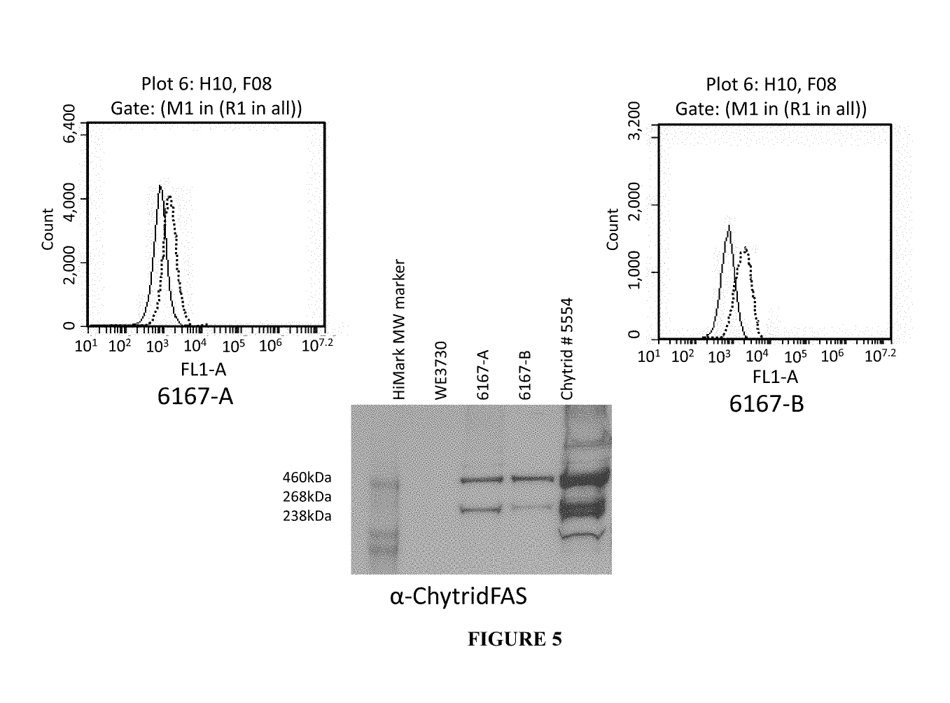

Two lines demonstrating fully penetrant ChytFAS expression by flow cytometry analysis were also assessed by Western for FAS protein expression (FIG. 5A) , using an antibody reactive against chytrid FAS for the ChytFAS transformants. Although 6167-B had a GFP fluorescence peak shifted farther to the right (at a higher fluorescence value) than the 6167-A GFP fluorescence peak was shifted, this difference was not reflected in the protein abundance as detected by Western blot (FIG. 5). Interestingly though, strain 6167-B demonstrated higher FAS activity in in vivo culture assays than did strain 6167-A, as described below.

### Example 3

### Fatty Acid Synthase Activity in Transformed Nannochloropsis lines

To analyze FAS activity in selected transformants, cell extracts of lines 6167-A and 6167-B expressing Chytrid FAS, and strains 6200-33, 6200-38, 6200-43, 6201-43, and 6201-48, all selected as demonstrating complete penetrance of DrFAS (FIGS. 3A and 3B), were assayed for FAS activity. Malonyl-CoA dependent NADPH oxidation measured at ABS 340 nm was determined on clarified, desalted extracts in triplicate.

Aliquots of cell cultures were pelleted and the pellets (approximately 200-400 μl packed volume) were resuspended in 2 ml of ice cold extraction buffer (50 mM HEPES pH 7.0 (or Tris pH 8.0), 100 mM KCl, 2 mM DTT (from fresh 1 M stock), 1 protease inhibitor cocktail from Roche at right concentration (e.g. 1 tablet for 10 ml). A similarly sized yeast pellet was treated the same way as a positive control extract.

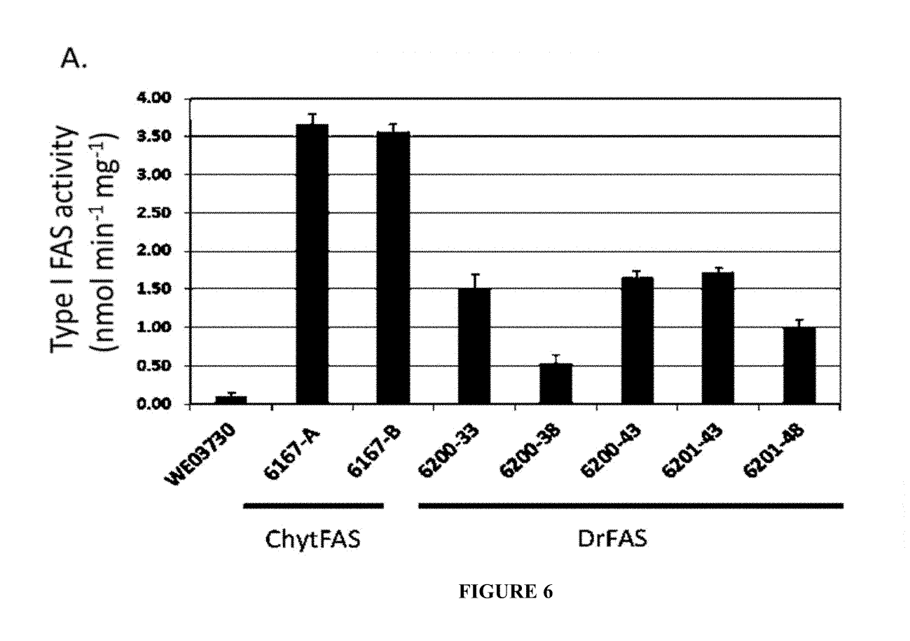

The resuspensions were transferred to a 2 ml screw cap vial containing approximately 500 μl bed volume of zirconium beads. The resuspension were bead beaten in a pre-chilled block 3 times for 1 minute to disrupt the cells. The lysed cells were centrifuged at 20,000×g at 4° C. for 20 minutes, and the supernatant and de-salted on Zeba mini-columns (Pierce, product 89882) after equilibration with extraction buffer (above). Protein concentration was measured with the Pierce BCA detection kit. The fatty acid synthase (FAS) assay was essentially according to the procedure of Lynen (1969) Meth Enzymol 14:17-33: a 2x buffer stock containing 0.2 M KH₂PO₄ pH 6.6, 2 mM EDTA and 0.6 mg/ml BSA was used to make a working stock assay consisting of: 0.1M KH₂PO₄ pH 6.6, 1 mM EDTA, 1 mM DTT, 40 μM Acetyl-CoA, 110 μM Malonyl-CoA (omitted in negative control assays.), 180 μM NADPH, and 1 mg/L BSA. 50 to 100 μg of total soluble protein from the extracts as prepared above were then added to each reaction mix. The change in absorbance at 340 nm per minute was measured and used to calculate the μmols oxidized NADPH per minute (FIG. 6).

Interestingly, the amount of activity demonstrated in the transformed lines correlated well with the degree to which the GFP fluorescence curves in the flow cytometry analysis were shifted to the right when assessing penetrance of GFP expression. Chytrid FAS transformed lines 6167-A and 6167-B were given strain named GE-6889 and GE-6890, respectively, and DrFAS transformed lines 6200-33 was given the strain name GE-6947, DrFAS transformed lines 6200-33 was given the strain name GE-6947, DrFAS transformed lines 6200-38 was given the strain name GE-6948, DrFAS transformed lines 6200-43 was given the strain name GE-6949, DrFAS transformed lines 6201-43 was given the strain name GE-6950, DrFAS transformed lines 6201-48 was given the strain name GE-6951.

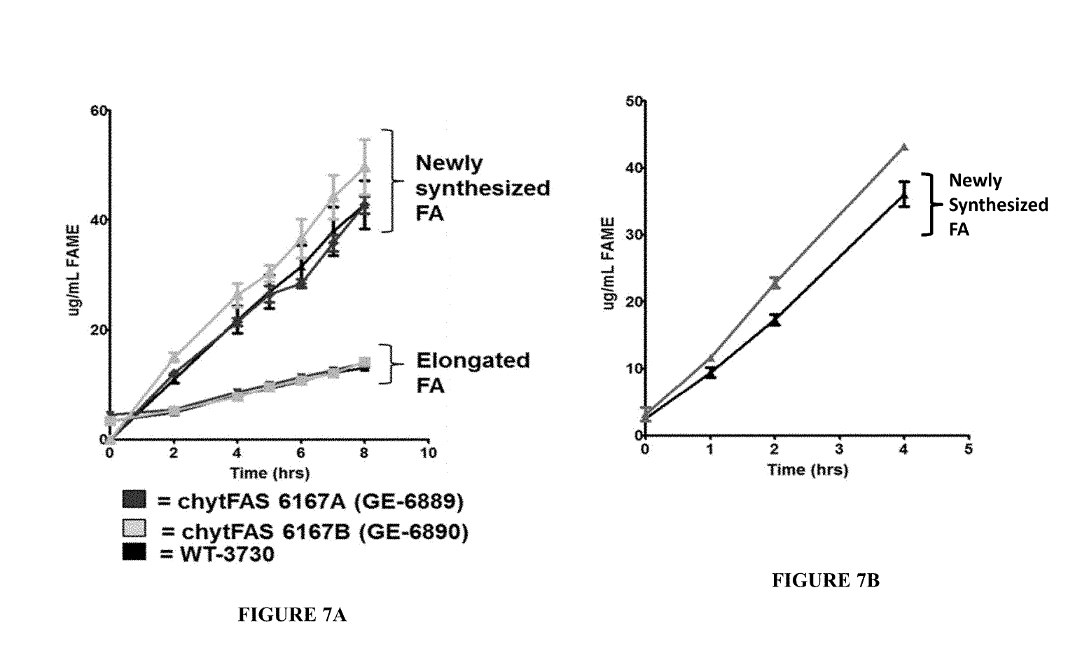

The lines were next analyzed for in vivo FAS rate determination under phototrophic and mixotrophic growth conditions with either ¹³C bicarbonate or ¹³C-labeled acetate added to the medium, respectively. Cultures (duplicates were run for each culture condition) were adapted to 16:8 light/dark cycles at ˜275 μE light (light limited growth) and grown to an OD₇₃₀ of approximately 3.0 in an Adaptis chamber. Prior to the onset of the photoperiod, cultures were centrifuged and resuspended (250 ml final vol.) to an OD₇₃₀ of 1.0 in PM074 medium buffered with 20 mM HEPES pH 7.4 and containing either 20 mM ¹³C bicarbonate (phototrophic) or 10 mM ¹³C sodium acetate (mixotrophic). Cultures were placed in front of an LED array supplying ˜275 μE light from one direction, and FAME samples were taken at 0, 1, 2, and 4 h from a 50 ml culture volume. FAME was analyzed essentially as described in U.S. Patent Application Publication US 2015/0191515, incorporated herein by reference. FIG. 7A shows that under photoautotrophic conditions where inorganic carbon was substantially the sole source of carbon in the culture medium, strain GE-6890, demonstrating fully penetrant expression of chytrid FAS (see FIG. 5, strain 6167-B), produced more newly synthesized fatty acids (represented as FAME) than controls. Newly synthesized fatty acids are fatty acids that show a high degree of labeling and have been synthesized de novo during the labeling experiment, where elongated fatty acids are C20:x fatty acids with one to four labeled carbons that arise from elongation of previously existing 16:x and 18:x fatty acids.

Strain GE-6890 is ChytFAS transformant line 6167-B whose penetrance profile showed a single peak shifted to the right with respect to wild type. Strain GE-6889, which is ChytFAS transformant line 6167-A, also demonstrated complete penetrance but the penetrance profile of GE-6889 (6167-A) in FIG. 5 showed a single peak that is not shifted as far to the right with respect to wild type as the fluorescence peak of GE-6890 (6167-B). Strain GE-6889 did not show any increase in FAME production over wild type in the radiolabeling experiment in which the strains are cultured using only an inorganic carbon source. However, when cultured under mixotrophic conditions, in which the cultures include an organic carbon source (10mM acetate) strain GE-6889 demonstrated increased fatty acid synthesis with respect to wild type cells, demonstrating that this fully penetrant strain, while demonstrating less activity than transformant GE-6890, did have increased FAS activity in mixotrophic conditions (FIG. 7B).

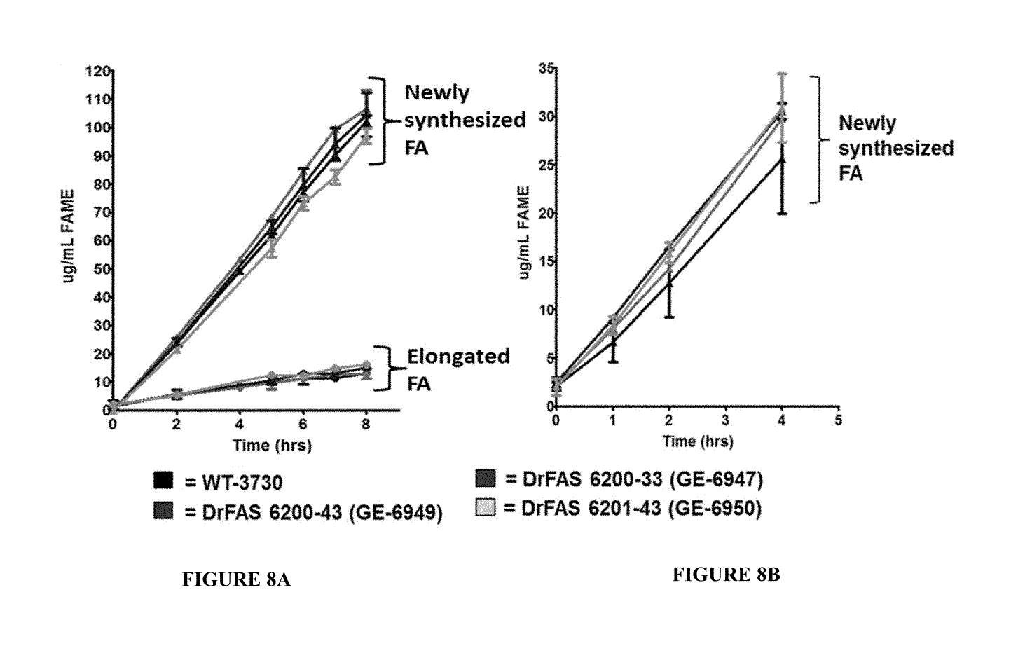

With respect to transformed strains expressing DrFAS, the same culture assay for FAS activity using under phototrophic and mixotrophic growth conditions with either ¹³C bicarbonate or ¹³C-labeled acetate added to the medium, respectively, was performed on cultures of fully penetrant strain GE-6947 (transformed line 6200-33), fully penetrant strain GE-6949 (transformed line 6200-43), and fully penetrant strain GE-6950 (transformed line 6201-43). These assays were performed exactly as detailed above, with duplicate cultures for each strain. FIG. 8A shows that while cytoplasmically expressed Type I FAS from Danio rerio did not increase photoautotrophic production of fatty acids, all three strains fully penetrant for expression of the heterologous Type I FAS construct produced more fatty acids (measured as FAME) than did wild type cells (FIG. 8B).

All references cited herein are incorporated by reference in their entireties. All headings are for the convenience of the reader and do not limit the invention in any way. References to aspects or embodiments of the invention do not necessarily indicate that the described aspects may not be combined with other described aspects of the invention or features of other aspects of the invention.

Although the invention has been described with reference to the above examples, it will be understood that modifications and variations are encompassed within the spirit and scope of the invention. Accordingly, the invention is limited only by the following claims.

1. A recombinant alga comprising an exogeous nucleic acid molecule that encodes a Type I fatty acid synthase (FAS).

2. A recombinant alga according to claim 1, wherein the Type I FAS has at least 80% identity to an animal, fungal, or labyrinthylomycete Type I FAS.

3. A recombinant alga according to claim 1, wherein the Type I FAS is an animal, fungal, or labyrinthylomycete Type I FAS having at least 50%, at least 55%, at least 60%, at least 65%, at least 70%, at least 75%, at least 80%, or at least 85%, for example at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to a Type I FAS selected from the group consisting of SEQ ID NO:3, SEQ ID NO:8, SEQ ID NO:19, SEQ ID NO:20, SEQ ID NO:22, and SEQ ID NO:24.

4. A recombinant alga according to claim 2, wherein the Type I FAS is an animal Type I FAS.

5. A recombinant alga according to claim 1, wherein the Type I FAS has at least 50%, at least 55%, at least 60%, at least 65%, at least 70%, at least 75%, at least 80%, or at least 85%, for example at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity with a fatty acid synthase selected from the group consisting of SEQ ID NO:3, SEQ ID NO:20, SEQ ID NO:22, and SEQ ID NO:24.

6. A recombinant alga according to claims 4, further comprising an exogenous nucleic acid sequence that encodes a pantetheine phosphotransferase (PPT).

7. A recombinant alga according to claim 6, wherein the PPT has at least 50%, at least 55%, of at least 70%, at least 65%, at least 70%, at least 75%, at least 80%, or at least 85%, for example at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity with a PPT selected from the group consisting of SEQ ID NO:6, SEQ ID NO:21, SEQ ID NO:23, and SEQ ID NO:25.

8. A recombinant alga according to claim 2, wherein the Type I FAS is a labyrinthulomycte Type I FAS.

9. A recombinant alga according to claim 8, wherein the Type I FAS is a Type I FAS of a Labryinthula, Labryinthuloides, Thraustochytrium, Schizochytrium, Aplanochytrium, Aurantiochytrium, Oblongichytrium, Japonochytrium, Diplophrys, or Ulkenia species.

10. A recombinant alga according to claim 8, wherein the Type I FAS has at least 50%, at least 55%, at least 60%, at least 65%, at least 70%, at least 75%, at least 80%, or at least 85%, for example at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity with SEQ ID NO:8 or SEQ ID NO:19.

11. A recombinant alga according to claim 1, wherein the recombinant alga has a higher rate of fatty acid synthesis as compared to a control alga substantially identical to the recombinant alga, with the exception that the control alga does not include an exogeous nucleic acid sequence that encodes a Type I FAS.

12. A recombinant alga according to claim 11, wherein the recombinant alga has a higher rate of fatty acid production than the control alga under nitrogen replete conditions.

13. A recombinant alga according to claim 1, wherein the recombinant alga further includes at least one exogenous gene that encodes a polyypeptide that participates in the synthesis of a fatty acid, fatty acid derivative, diacylglyceride, or triacylglyceride.

14. A recombinant alga according to claim 13, wherein the recombinant alga further includes at least one exogenous gene that encodes an acyl-ACP thioesterase, a 4-hydroxybenzoyl-CoA thioesterase, an acyl-CoA synthetase, an acyl-CoA reductase, a carboxylic acid reductase, an acyl-ACP reductase, a fatty aldehyde reductase, a wax synthase, a fatty acid decarboxylase, a fatty aldehyde decarbonylase, an acetyl-CoA carboxylase, a malonyl CoA: ACP transacylase, a beta-ketoacyl-ACP synthase, an acyl-ACP thioesterase, an acyl-CoA thioesterase, a polypeptide having lipolytic activity, a glycerolphosphate acyltransferase (GPAT), a lysophosphatidic acid acyltransferase (LPAAT), a phosphatidic acid phosphatase (PA), or a diacylglycerol 0-acyltransferase (DGAT).

15. A recombinant alga according to claim 1, wherein the recombinant alga is of a genus selected from the group consisting of Achnanthes, Amphiprora, Amphora, Ankistrodesmus, Asteromonas, Boekelovia, Bolidomonas, Borodinella, Botrydium, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Desmodesmus, Dunaliella, Elipsoidon, Emiliania, Eremosphaera, Ernodesmius, Euglena, Eustigmatos, Franceia, Fragilaria, Fragilaropsis, Gloeothamnion, Haematococcus, Hantzschia, Heterosigma, Hymenomonas, Isochrysis, Lepocinclis, Micractinium, Monodus, Monoraphidium, Nannochloris, Nannochloropsis, Navicula, Neochloris, Nephrochloris, Nephroselmis, Nitzschia, Ochromonas, Oedogonium, Oocystis, Ostreococcus, Parachlorella, Parietochloris, Pascheria, Pavlova, Pelagomonas, Phceodactylum, Phagus, Picochlorum, Platymonas, Pleurochrysis, Pleurococcus, Prototheca, Pseudochlorella, Pseudoneochloris, Pseudostaurastrum, Pyramimonas, Pyrobotrys, Scenedesmus, Schizochlamydella, Skeletonema, Spyrogyra, Stichococcus, Tetrachlorella, Tetraselmis, Thalassiosira, Tribonema, Vaucheria, Viridiella, Vischeria, and Volvox.

16. A method of producing a lipid comprising culturing a recombinant alga according to claim 1 to produce a lipid.

17. A method according to claim 16, further comprising recovering the lipid from the cells, culture medium, or whole culture.

18. A method according to claim 16, wherein the culturing is under nitrogen replete conditions.

19. A method according to claim 16, wherein the lipid comprises a fatty acid or a fatty acid derivative.

20. A method according to claim 16, wherein the lipid comprises a diacylglyceride or a triacylglyceride.
